(19) United States
(12) Patent Application Publication
(10) Pub. No.: US 2008/0113358 A1
(43) Pub. Date: May 15, 2008

(54) SELECTION OF CELLS USING BIOMARKERS

(76) Inventors: Ravi Kapur, Stoughton, MA (US); Mehmet Toner, Wellesley Hills, MA (US); Zihua Wang, Newton, MA (US)

Correspondence Address: WILSON SONSINI GOODRICH & ROSATI, 650 PAGE MILL ROAD, PALO ALTO, CA, 94304-1050, US

(21) Appl. No.: 11/830,546

(22) Filed: Jul. 30, 2007

Related U.S. Application Data
(60) Provisional application No. 60/820,778, filed on Jul. 28, 2006.

(51) Int. Cl. C12Q 1/68

(52) U.S. Cl. 435/6

(57) ABSTRACT

The present invention provides systems, apparatuses, and methods to isolate, select or detect the presence of a target cell (e.g., fetal cells) in a sample comprising mixed populations of cells that vastly outnumber the target cells. Target cells include fetal cells, such as nucleated red blood cells, and methods of selecting such cells include diagnosis of fetal abnormalities, i.e., aneuploidy. Furthermore, methods comprise utilizing fetal biomarkers to select fetal cells in a sample comprising fetal and adult cells.

### RELATED APPLICATIONS

This application claims the benefit of priority to U.S. Provisional Application No. 60/820,778, filed Jul. 28, 2006, which is incorporated by reference herein in its entirety. In addition, this application is related to the following copending patent applications: application Ser. No. 11/763,426 [Attorney Docket No 32047.717.201]; application Ser. No. 11/763,133 [Attorney Docket No 32047.718.201]; application Ser. No. 11/763,245 [Attorney Docket No 32047.719.201] application Ser. No. 11/763,431 [Attorney Docket No 32047.720.201]; and application Ser. No. 11/763,421 [Attorney Docket No 32047.722.201]; which are incorporated herein by reference in their entirety.

### BACKGROUND OF THE INVENTION

Analysis of specific cells can give insight into a variety of diseases. For instance, social developments have resulted in an increased number of prenatal tests. However, obtaining the necessary sample component for conducting such analysis for a particular disease or condition is not always safe or effective. For example, current methods of amniocentesis and chorionic villus sampling (CVS) are potentially harmful to the mother and to the fetus. The rate of miscarriage for pregnant women undergoing amniocentesis is increased by 0.5-1%, and that figure is slightly higher for CVS. Because of the inherent risks posed by amniocentesis and CVS, these procedures are offered primarily to older women, i.e., those over 35 years of age, who have a statistically greater probability of bearing children with congenital defects. As a result, a pregnant woman at the age of 35 has to balance an average risk of 0.5-1% to induce an abortion by amniocentesis against an age related probability for trisomy 21 of less than 0.3%.

To eliminate the risks associated with invasive prenatal screening procedures, non-invasive tests for detection, diagnosis and prognosis of diseases, have been utilized. For example, maternal serum alpha-fetoprotein, and levels of unconjugated estriol and human chorionic gonadotropin are used to identify a proportion of fetuses with Down's syndrome. Unfortunately, these tests are not accurate (e.g., result in unreasonable number of false positives). Similarly, ultrasonography is used to determine congenital defects involving neural tube defects and limb abnormalities, but is useful only in late pregnancy (e.g., fifteen weeks' gestation).

The presence of fetal cells in maternal circulation offers the opportunity to develop a prenatal diagnostic that obviates the risk associated with invasive diagnostics procedures. However, fetal cells are rare in maternal circulation, with estimated frequencies of about 1 to 6 cells/1 ml (e.g., about 1 fetal cell to about 10⁶-10⁷ maternal nucleated cells). Therefore, any proposed analysis of fetal cells to diagnose fetal abnormalities requires enrichment and selection of fetal cells. Enriching fetal cells from maternal peripheral blood is challenging, time intensive and any analysis derived therefrom is prone to error. As such, there remains a great need for development of a noninvasive, specific and sensitive method that can be applied in diagnostics, including prenatal diagnostics.

### SUMMARY OF THE INVENTION

The methods disclosed herein allow for the selection of a target cell or cell component with high sensitivity and specificity from a sample comprising a mixed population of cells containing relatively rare amount of such target cell or components. In various embodiments, size-based separation and/or biochemical manipulations are utilized to select a target cell or cell component.

The invention generally features methods and compositions for identifying and isolating rare cells or nuclei of interest from a mixed sample comprising two or more population of cells or cell components utilizing enrichment and specific selection of a cell or cell component which can be subsequently screened for genetic disorders. In one aspect of the invention a biological sample is treated with systems providing a microfluidic platform and process flow to remove unwanted cell populations from the sample.

In some embodiments, such systems are utilized to remove enucleated red blood cells (RBC) or white blood cells (WBC) from a sample. In one embodiment a sample is administered to a cell separation module (CSM) to remove RBCs and Magnetic Hemoglobin Enrichment Module (MHEM) to remove WBCs. In some embodiments, the sample is a blood sample, having a volume that can be in various mL range, such as from 1 ml to 100 ml.

In various embodiments a CSM of the invention can separate a first and second cell or cell component present in a sample to that is assayed. For example, a CSM can provide size-based separation of a first component of a mixed sample (e.g., fetal nucleated red blood cells (fnRBC)) and a second component of the mixed sample (e.g. enucleated maternal red blood cells). In some embodiments, the first component comprises cells or cell components that are larger than a critical size, and the second component comprises cells or cell components that are smaller than a critical size. In some embodiments, the cells or cell components of the first component are directed by the CSM in a first direction and the cells or cell components of the second component are directed by the CSM in a second direction. In some embodiments, the first and/or second directions may lead to separate exit ports for the first and second components.

A CSM can comprise one or more arrays of obstacles, wherein an array can have obstacles of the same or different shape. In various embodiments, obstacles can have shapes including but not limited to diamonds, hexagons, octagons, rectangles, squares, circles, semi-circles, triangles and ellipses. In one embodiment CSM can comprise one or more arrays of obstacles that form a network of gaps (FIG. 4). Such obstacles can vary in post diameter, spacing, periodicity, width, height to produce varying fluid flow rates and interaction with cells or cellular components present in a sample. Such array formats can be manufactured using methods familiar to the artisan including but not limited to lasering, micromachining, photolithography, reactive ion etching, ion beam milling, embossing, electroforming, electroplating, injection molding, compression molding, casting, or reaction injection molding.

A MHEM (may also referred to as “HE” herein) can be utilized to separate white blood cells from other cell populations present in a sample. In one embodiment, the sample is treated so as to convert diamagnetic hemoglobin to paramagnetic methemoglobin, thus facilitating separation of methemoglogin-containing cells by utilizing a magnetic field. In one embodiment, such a conversion treatment comprises treating a sample with a compound (e.g., sodium nitrite) that oxidizes an iron prosthetic group of hemoglobin (as well as other iron-containing proteins) from ferrous to ferric iron, thereby magnetizing or rendering magnetically responsive, cells or cell components containing ferric ion (hemoglobin).

In one embodiment, a maternal blood sample is administered to systems disclosed herein (e.g., (CSM, MHEM, etc.). For example, the blood sample is applied to the CSM, which may enrich for all nucleated cells (e.g. enucleated RBCs are separated from nucleated cells). Such enrichment may be followed by capturing all or substantially all hemoglobin-containing cells using an MHEM, resulting in isolation and enrichment of nucleated red blood cells (nRBCs) from whole blood in high numbers and high purity.

In some embodiments, such enriched nRBCs or cellular components therefrom are arrayed on a substrate, prior to identification or further analysis. In one embodiment, such cellular components include nuclei. In some embodiments arrays can comprise cells that grow on a substrate or cells that have been cytospun onto a substrate (e.g., slide). Cells can be treated to remove enucleated RBC (e.g., cell lysis) or can be cytospun onto a substrate. Thus in one embodiment, a nuclei array is disposed on slides or a porous ceramic substrate. Such arrays can be subjected to assays for clinical study.

In one aspect, the invention features a method for imaging cellular components from cells or nuclei of interest to differentiate these cells or nuclei from other cells or nuclei. Therefore, individual cells or nuclei are identified as fetal cell or fetal cell nuclei. The method may further include simultaneously imaging multiple cellular components to facilitate the target cells or nuclei identification and determination of the presence or absence of chromosomal aneuploidy such as but not limited to autosomal trisomy, sex chromosome abnormalities, triploidy, including chromosome 21, 18, and/or 13 trisomy. Thus the identified target cells or nuclei can be further subjected to analysis to obtain genetic information (e.g., identify aneuploidy). In various embodiments genetic information obtained provides disease status, gender, paternity, aneuploidy, or presence or absence of a mutation.

Another aspect of the invention is directed to determining whether a cell is a fetal or maternal cell (or cell component thereof) by enriching for the desired cell or cell component. Thus, cells or cell components are distinguished based on their origin as maternal or fetal. For example, in one embodiment, fetal nRBCs are distinguished from maternal nRBCs based on differential expression (fetal to maternal) of marker genes. In one embodiment, differential expression comprises imaging hemoglobin γ, α or β expression. Differential expression can be quantified utilizing techniques for nucleic acid measurement (e.g., PCR, RNA-FISH) and includes measuring ribosomal RNA, mRNA, nascent or unspliced RNA.

Thus fetal nuclei thus identified can be subject to genetic analysis (e.g., chromosomal FISH) or plucked (e.g., microdissection) for further diagnostic genetic analysis (e.g., STR assay) to determine if fetal cells are normal or comprise a disorder (e.g., aneuploidy). In another embodiment genetic analysis is conducted concurrent to/with determination of the cell or cell component as fetal or maternal. For example, probes for diagnostic as well as for identification are used to stain enriched cells or cell components, thereby determining the cell or cell component origin (e.g., fetal, maternal or paternal) as well as assaying for a disorder. Thus, a positive result is visualized for a fetal cell and concurrently such fetal cell is stained to determine if a genetic disorder is present.

In various embodiments of the invention, the cell of interest is a fetal red blood cell, a cancer cell, pathogen, connective tissue cell, or immune cell. A cellular component can be ribosomal RNA, mRNA, nascent or unspliced RNA. The methods of RNA imaging include in situ fluorescence hybridization (RNA-FISH), in situ reverse transcription PCR, nucleic acid based amplification such as ligase chain reaction (LCR), transcription mediated amplification (TMA), strand displacement amplification (SDA), rolling-circle amplification (RCA), multiple-displacement amplification (MDA), and helicase-dependent amplification (HDA).

In some embodiments, determining abnormalities involves quantifying one or more regions of genomic DNA regions from the mixed sample and to determine the presence of a fetal abnormality. Preferably, such regions are polymorphic e.g., STR regions.

Non limiting examples of fetal abnormalities that can be determined by quantifying regions on one or more chromosomes include trisomy 13, trisomy 18, trisomy 21 (Down Syndrome), Klinefelter Syndrome (XXY) and other irregular number of sex or autosomal chromosomes. Furthermore, the methods herein can distinguish maternal trisomy from paternal trisomy, as well as total aneuploidy from segmental aneuploidy. Therefore, in some embodiments, the maternal or paternal origin of the fetal abnormality can be determined. In some embodiments, total or segmental aneuploidy may be haploidy, triploidy, tetraploidy, pentaploidy and other atypical multiples of normal chromosome number.

In various embodiments DNA obtained from fetal cells identified using methods/devices of the invention are subsequently subjected to various molecular biology and biochemical analysis. For example the genomic DNA region(s) can be quantified by amplifying the regions using, for example, PCR, or preferably quantitative PCR. Alternatively, quantification of the regions can be achieved using capillary gel electrophoresis (CGE). In some embodiments, total genomic DNA is pre-amplified prior to the quantitative amplification step to increase the overall abundance of DNA. Such pre-amplification step can involve the use of multiple displacement amplification.

In some embodiments the genomic DNA regions quantified can be in one chromosome, or in some other embodiments in 2 or more chromosomes. The polymorphic regions can be quantified on either or both sex chromosomes X and Y, and on autosomal chromosomes including chromosomes 13, 18 and 21.

### SUMMARY OF THE DRAWINGS

The novel features of the invention are set forth with particularity in the appended claims. A better understanding of the features and advantages of the present invention can be obtained by reference to the following detailed description that sets forth illustrative embodiments, in which the principles of the invention are utilized, and the accompanying drawings of which:

FIGS. 1A-1D illustrate various embodiments of the invention.

FIG. 2 illustrates an antibody capture device.

FIG. 3 illustrates a size based separation module or CSM.

FIG. 4 illustrates two outputs of a size based separation module.

FIG. 5 illustrates one embodiment for magnetic hemoglobin enrichment module (MHEM) processing.

FIG. 6 illustrates staining of a sample processed by CSM and MHEM.

FIG. 7 illustrates graphs depicting expression of gamma v. beta globin in fetal v. maternal nRBCs.

FIG. 8 illustrates graphs for gamma v. beta globin expression for 1, 2, and 40 nuclei.

FIG. 9 illustrates a schematic for one embodiment of the invention.

FIG. 10 illustrates a schematic for one embodiment of the invention.

FIG. 11 illustrates a flow through diagram for steps to confirm fetal origin using STR.

FIG. 12 illustrates gamma/beta ratios obtained from 1, 2 and 40 nuclei.

FIG. 13 illustrates simultaneous detection of maternal and paired fetal-maternal mixture analysis to confirm fetal cells are present on slides.

FIG. 14 illustrates different shape obstacles.

FIGS. 15A-D illustrate flow directions and gap width for an array.

FIGS. 16A-C illustrate one embodiment of an affinity separation module.

FIG. 17 illustrates one embodiment of a magnetic separation module.

FIG. 18 illustrates typical locus patterns arising from a normal (diploid) fetus and mother.

FIG. 19 illustrates typical locus patterns arising from trisomic fetal cells.

FIGS. 20A-D illustrates various embodiments of a size-based separation module.

FIGS. 21A-B illustrate cell smears of the product and waste fractions.

FIGS. 22A-F illustrates cell isolated fetal cells confirmed by the reliable presence of male Y chromosome

FIG. 23 illustrates trisomy 21 pathology in an isolated fetal nucleated red blood cell.

FIG. 24 depicts a flow chart depicting the major steps involved in detecting paternal alleles in a fetal enriched sample using fluorescently labeled primers.

FIG. 25 illustrates a table with STR loci that can be used for fetal detection.

FIG. 26 illustrates a table with exemplary external primers for STR loci.

FIG. 27 illustrates a table with exemplary internal primers for STR loci.

FIG. 28 illustrates the resolution for the ABI 310 bioanalyzer.

FIG. 29 illustrates the detection limit on fixed cord blood.

FIG. 30 illustrates the detection of 10 fetal cells at 10% purity without nested PCR.

FIG. 31 illustrates the generation of STR markers on fixed cells recovered from a slide.

FIG. 32 illustrates detection of fetal alleles at less than 10% purity after nested PCR amplification of STRs.

FIG. 33 illustrates one embodiment for a magnet comprised in a MHEM.

FIG. 34A-C illustrates histograms generated by a hematology analyzer from (A) a blood sample, (B) a waste sample and (C) a product fraction.

FIG. 35A-D illustrates one embodiment of a mask employed to fabricate a size-based separation device.

FIG. 36A-D illustrates one embodiment of a mask employed to fabricate a size-based separation device.

FIG. 37A-D illustrates one embodiment of a mask employed to fabricate a size-based separation device.

FIG. 38A-G is an SEM of a size-based separation module.

FIG. 39A-D illustrates one embodiment of a mask employed to fabricate a size-based separation device.

FIG. 40A-F is an SEM of a size-based separation module.

FIG. 41A-F is an SEM of a size-based separation module.

FIG. 42A-D illustrates one embodiment of a mask employed to fabricate a size-based separation device.

FIG. 43A-S is an SEM of a size-based separation module.

FIG. 44A-C is an SEM of a size-based separation module.

FIG. 45 illustrates experimental results where CGH is used to detect aneuploidy in a sample with 100 genome copies.

FIG. 46 illustrates experimental results where CGH is used to detect aneuploidy in a sample with 10 genome copies.

FIG. 47 illustrates methods of fetal diagnostic assays. Fetal cells are isolated by CSM-HE enrichment of target cells from blood. The designation of the fetal cells may be confirmed using techniques comprising FISH staining (using slides or membranes and optionally an automated detector), FACS, and/or binning. Binning may comprise distribution of enriched cells across wells in a plate (such as a 96 or 384 well plate), microencapsulation of cells in droplets that are separated in an emulsion, or by introduction of cells into microarrays of nanofluidic bins. Fetal cells are then identified using methods that may comprise the use of biomarkers (such as fetal (gamma) hemoglobin), allele-specific SNP panels that could detect fetal genome DNA, detection of differentially expressed maternal and fetal transcripts (such as Affymetrix chips), or primers and probes directed to fetal specific loci (such as the multi-repeat DYZ locus on the Y-chromosome). Binning sites that contain fetal cells are then be analyzed for aneuploidy and/or other genetic defects using a technique such as CGH array detection, ultra deep sequencing (such as Solexa, 454, or mass spectrometry), STR analysis, or SNP detection.

FIG. 48 illustrates methods of fetal diagnostic assays, further comprising the step of whole genome amplification prior to analysis of aneuploidy and/or other genetic defects.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

Generally, the systems and methods of the invention enable isolation and analysis of target cells, such as fetal cells, from a mixed cell sample. The systems and methods herein can be used to identify fetal cells and optionally detect fetal aneuploidy even in a mixed sample wherein more than 50% of the cells and/or cellular components are non fetal cells and/or cellular components. In various embodiments, isolated target cells can be utilized for genetic testing (e.g., prenatal diagnostics, paternity testing or determination of genetic traits). Thereby, a novel method for non-invasive diagnostics is disclosed herein.

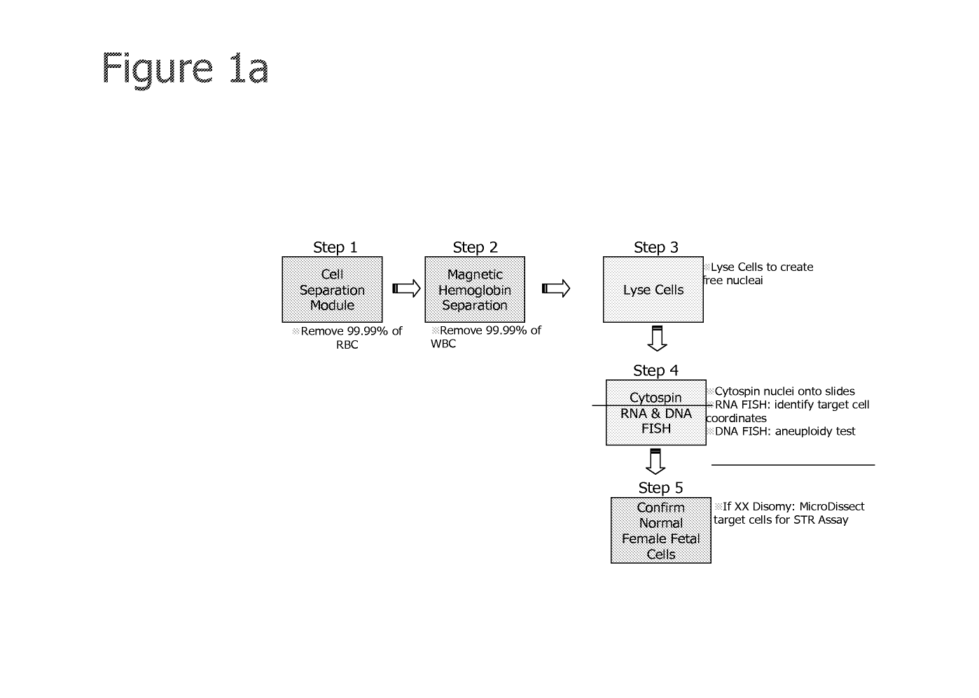

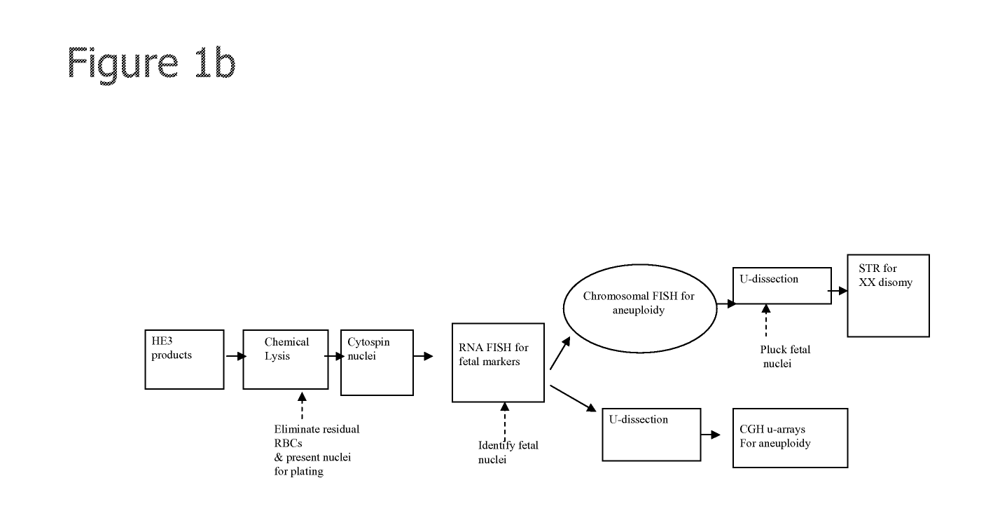

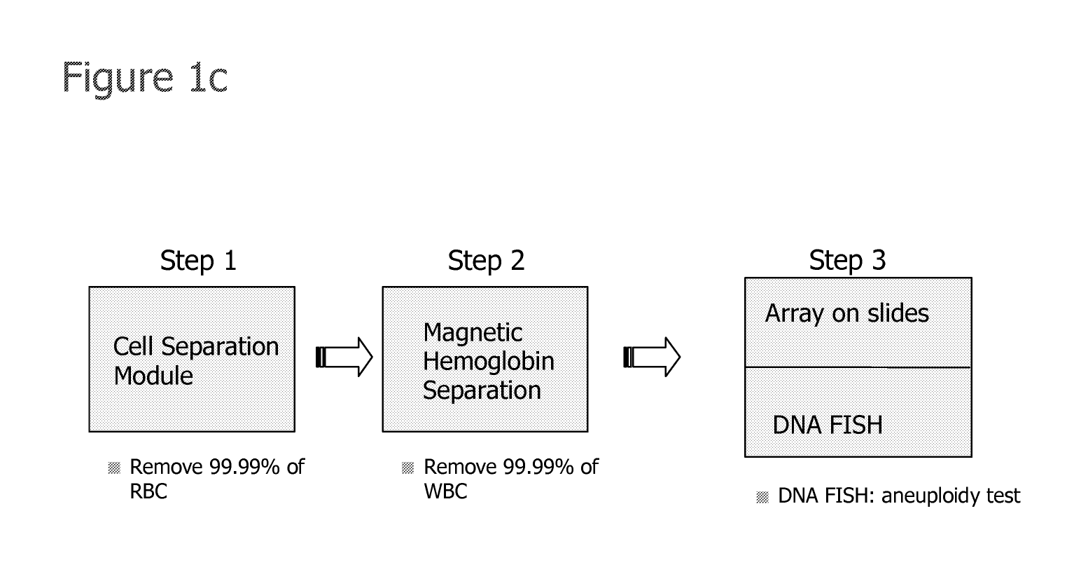

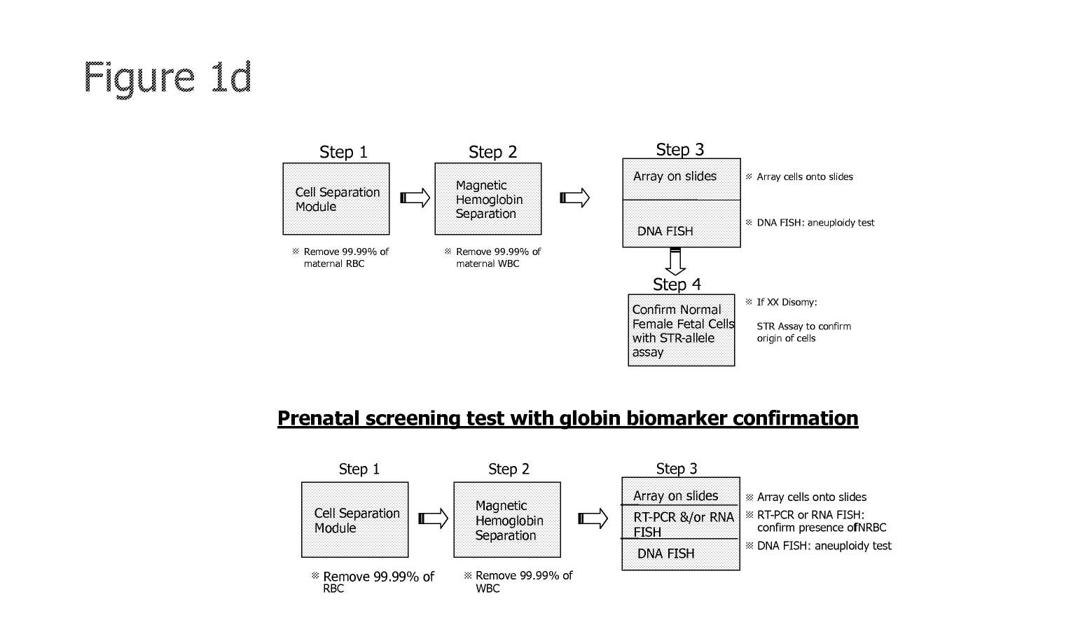

In one embodiment, as illustrated by FIG. 1a, a blood sample is obtained from a pregnant woman (e.g., first or second trimester of pregnancy). In step 1a, the blood sample is applied to a size-based cell separation module as further described herein, which removes up to 90, 91, 92, 93, 94, 95, 96, 97, 98, 99, 99.4, 99.5, 99.6, 99.7, 99.8, 99.9 or 99.99% of enucleated RBCs resulting in an output sample that is comprised of a mixed population of cells comprising white blood cells, fnRBC or both.

In step 2 of FIG. 1a, the enriched sample is treated with sodium nitrite which oxidizes an iron prosthetic group of hemoglobin (as well as other iron-containing proteins) from ferrous to ferric iron, thus utilizing an intrinsic property of cells in applying a high gradient magnetic field in a magnetic hemoglobin enrichment module (MHEM) which removes up to, 91, 92, 93, 94, 95, 96, 97, 98, 99, 99.4, 99.5, 99.6, 99.7, 99.8, 99.9 or 99.99% of white blood cells, e.g., hemoglobin-containing cells, resulting in an enriched output sample comprising nucleated red blood cells (nRBCs) from whole blood in high numbers and high purity.

In step 3 of FIG. 1a, cells enriched from the MHEM are treated with cell lysis buffer (e.g. CB buffer; Example 1) to release nuclei, as well as eliminate residual RBCs and present nuclei for plating.

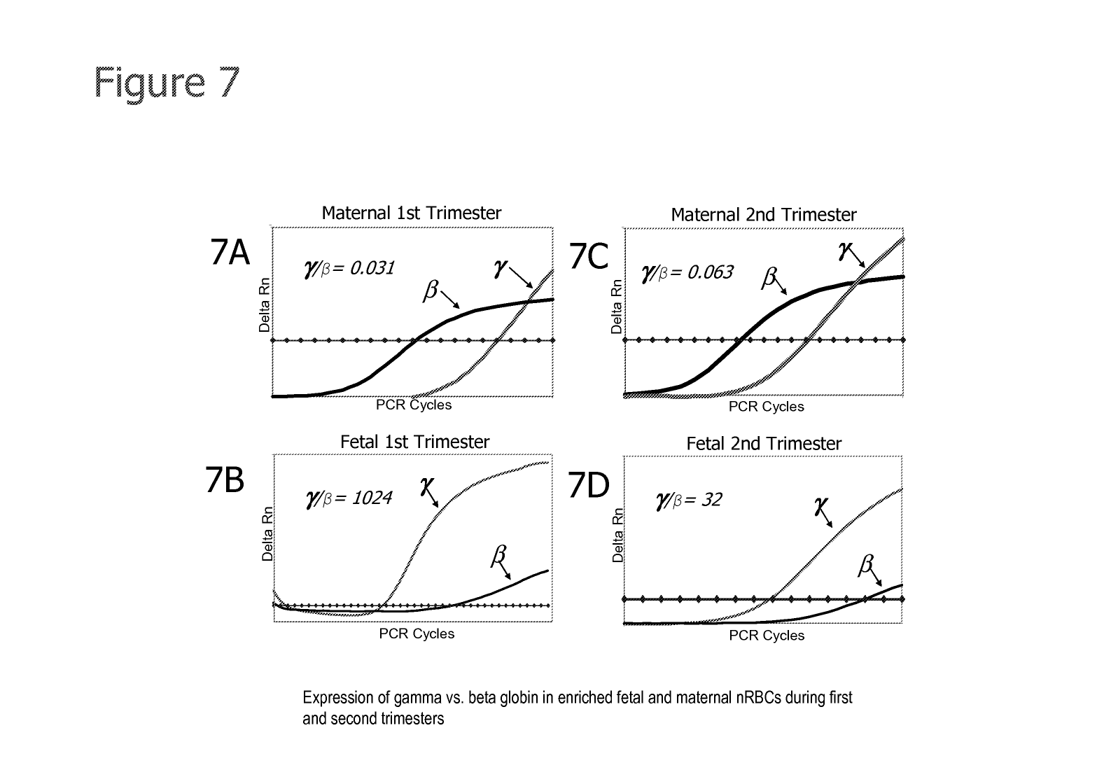

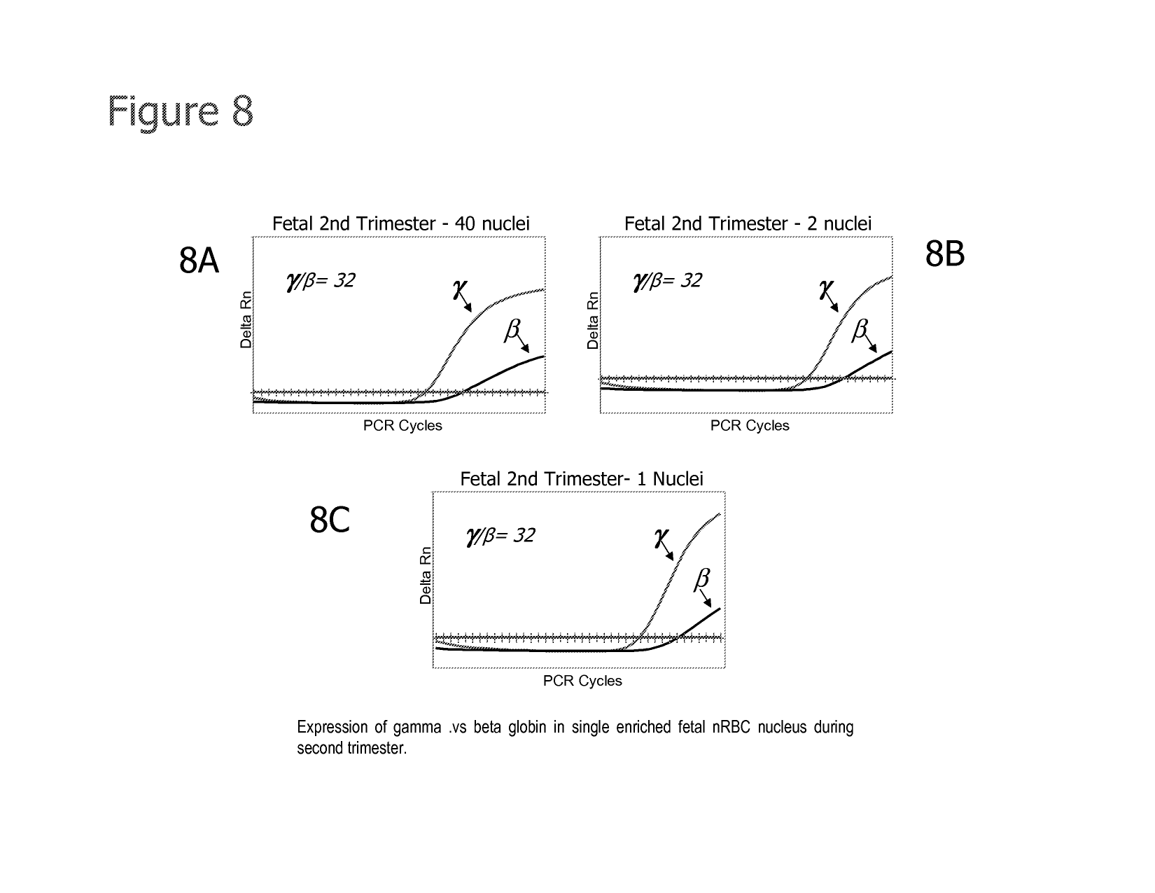

In step 4 of FIG. 1a, nuclei are cytospun onto slides (e.g., Example 3) using methods that are conventional in the art, such as disclosed in U.S. Pat. No. 6,821,732; 6,797,471; 6,780,592 or 6,291,163. Slides are then prepared for RNA-FISH (or RT-PCR) and/or DNA FISH. RNA FISH allows for the determination of expression levels for fetal biomarkers (e.g., gamma and beta). RNA-FISH can utilize probes disclosed herein that target intron-exon gaps so that nuclear transcripts are targeted. Methods of conducting FISH, including RNA FISH are conventional in the art, such as disclosed in U.S. Pat. No. 5,817,462. Alternatively, RT-PCR can be conducted to measure expression levels using the same or different probe/primer sequences. Methods of RT-PCR are also known in the art. Therefore, by measuring the ratio of gamma/beta hemoglobin, it can be determined whether the nuclei are from fetal or adult NRBCs (FIGS. 7 and 8). DNA-FISH (e.g., using probes to identify aneuploidy or to determine sex) can also be performed in this step to diagnose fetal abnormality (e.g., trisomy of chromosome 13, 18, 21, X). (e.g., Example 2 for preparation of probes; “Hybridization”, infra, Examples).

In step 5 of FIG. 1a, nuclei microdissection can be used to pluck fetal nuclei or a fetal nucleus from the slide. Microdissection is a conventional method, such as disclosed in U.S. Pat. No. 6,821,732; 6,797,471; 6,780,592 or 6,291,163. Generally, in microdissection a laser beam focally activates a special transfer film which bonds specifically to cells or nuclei identified and targeted by microscopy (e.g., RNA-FISH identification via biomarkers). The transfer film with the bonded cells is then lifted off the thin tissue section, leaving all unwanted cells behind (which would otherwise contaminate the molecular purity of subsequent analysis). The transparent transfer film is applied to the surface of the tissue section. Under the microscope, the diagnostic pathologist or researcher views the thin tissue section through the glass slide on which it is mounted and chooses microscopic clusters of cells or nuclei to study. When the cells of choice are in the center of the field of view, the operator pushes a button which activates a near IR laser diode integral with the microscope optics. The pulsed laser beam activates a precise spot on the transfer film immediately above the cells of interest. At this precise location the film melts and fuses with the underlying cells of choice. When the film is removed, the chosen cell(s) or nucleus (nuclei) are tightly held within the focally expanded polymer, while the rest of cells or nuclei are left behind. This allows multiple homogeneous samples within the nuclei array or cytological preparation to be targeted and pooled for extraction of molecules and analysis.

In alternative embodiments illustrated in FIG. 1b, nuclei are plucked (via microdissection) after the DNA-FISH step above, to conduct STR analysis to verify the RNA-FISH identification of fetal nuclei. Other genetic analyses to which the nuclei can be subjected include comparative genomic hybridization (CGH), SNPs analysis, or short tandem repeat analysis (STR).

### Sample Collection/Preparation

Samples can be obtained from an animal suspected of being pregnant, or that has been pregnant to detect the presence of a fetus or fetal abnormality. Such animals can be a human or a domesticated animal such as a cow, chicken, pig, horse, rabbit, dog, cat, or goat. Samples derived from an animal or human can include, e.g., whole blood, sweat, tears, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid. In one embodiment, where the sample to be tested or analyzed is a mixed sample (e.g. maternal blood sample), it is enriched for rare cells or rare cell components (e.g. fetal cells, fetal DNA or fetal nuclei including DNA) using one or more methods disclosed herein. Such enrichment increases the ratio of fetal cells to non-fetal cells; the concentration of fetal DNA to non-fetal DNA; or the concentration of fetal cells in volume per total volume of the mixed sample. For example, in one embodiment, enrichment comprises administering a sample to a CSM and HMEM, wherein the sample is a maternal blood sample to enrich at least one fetal cell.

To obtain a blood sample, any technique known in the art may be used, e.g. a syringe or other vacuum suction device. A blood sample can optionally be pre-treated or processed prior to enrichment. Examples of pre-treatment include the addition of a reagent such as a stabilizer, a preservative, a fixant, a lysing reagent, a diluent, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent or a combination thereof.

For example, when a blood sample is obtained, a preservative such an anti-coagulation agent and/or a stabilizer can be added to the sample prior to enrichment. This allows for extended time for analysis/detection. Thus, a sample, such as a blood sample, can be enriched and/or analyzed under any of the methods and systems herein within 1 week, 6 days, 5 days, 4 days, 3 days, 2 days, 1 day, 12 hrs, 6 hrs, 3 hrs. 2 hrs, or 1 hr from the time the sample is obtained. In various embodiments, a sample can have a volume that is about 1, 2, 3, 4, 5, 6, 7, 8, 9, 10, 11, 12, 13, 14, 15, 16, 17, 18, 19, 20, 25, 30, 35, 40, 45, 50, 55, 60, 70, 80, 90 or 100 mL. In some cases a 20 or 40 mL sample of peripheral blood is obtained from a female subject and is diluted, e.g., by combining it with another 20 or 40 mL of a diluent prior to enrichment, using a CSM and/or HMEM system of the invention.

### Sample Enrichment

Size Based Separation Enrichment

The samples herein can be enriched for one or more cells of interests or components thereof (e.g., fetal cells or fetal nuclei) using, for example, and/or a cell separation module (CSM).

A CSM is well suited to the isolation of rare cells from a heterogeneous blood sample according to size. In one embodiment the CSM fractionates blood cells and soluble plasma proteins based on “hydrodynamic size” under laminar flow conditions. Hydrodynamic size is a complex function of cell volume, shape and mechanical compliance. A CSM includes one or more precisely designed arrays of microposts that allow deterministic lateral displacement of components of fluids. The device can operate under laminar flow conditions, which provide a high degree of enrichment while limiting mechanical stress on cells and preventing cell lysis or intracellular activation. The CSM deflects blood cells above a critical hydrodynamic size—including most nucleated cells and some contaminating RBCs—into a buffer collection stream, while most RBCs, platelets, and serum proteins are collected in a separate waste fraction.

In various embodiments, sample flow rate is varied based on the configuration of a CSM of the invention, as well as the means for sample flow (e.g., gravity, pumped, etc.). In some embodiments, the sample flow rate is from about less than 0.001 mL/hr to about greater than 10 mL/hr. In various embodiments, the flow rate can be from about 0.250, 0.5, 0.75, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 14.0 to about 15.0 mL/hr.

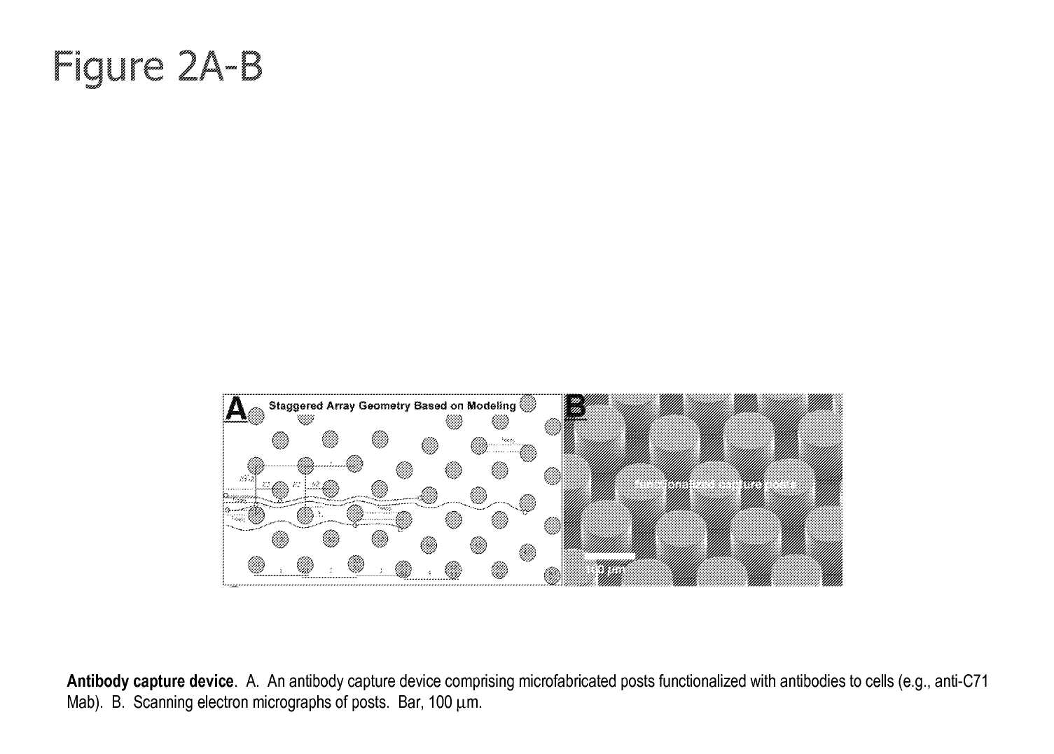

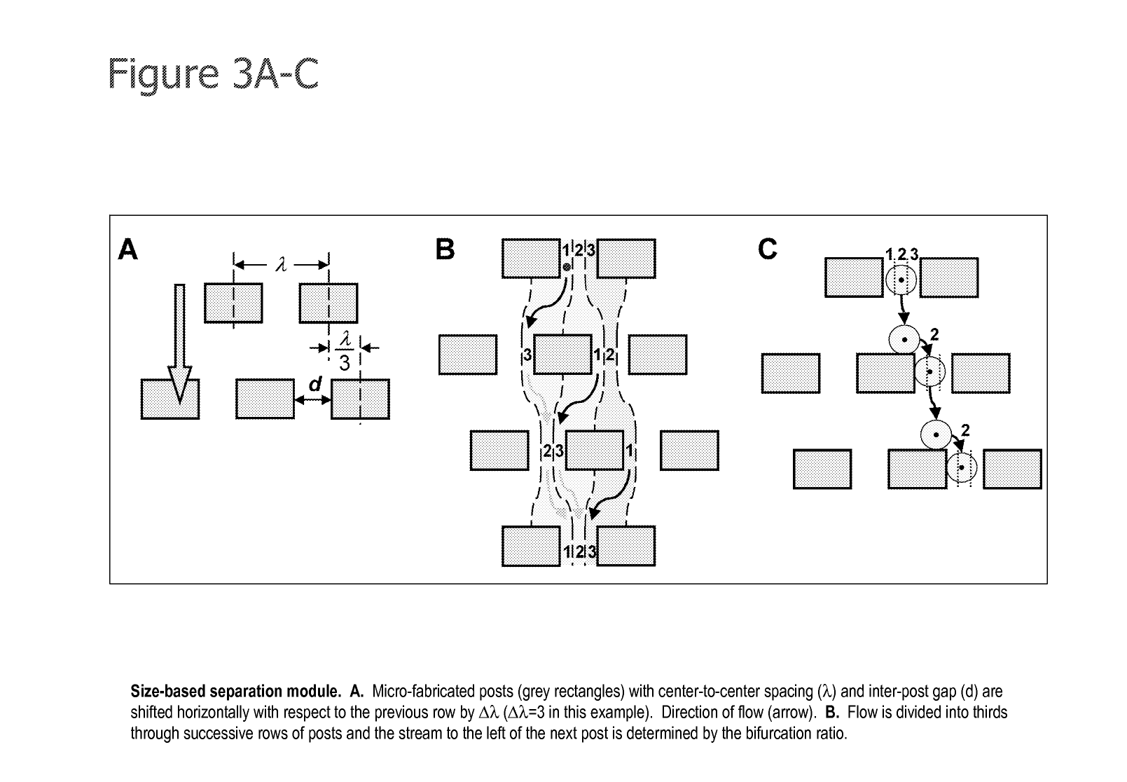

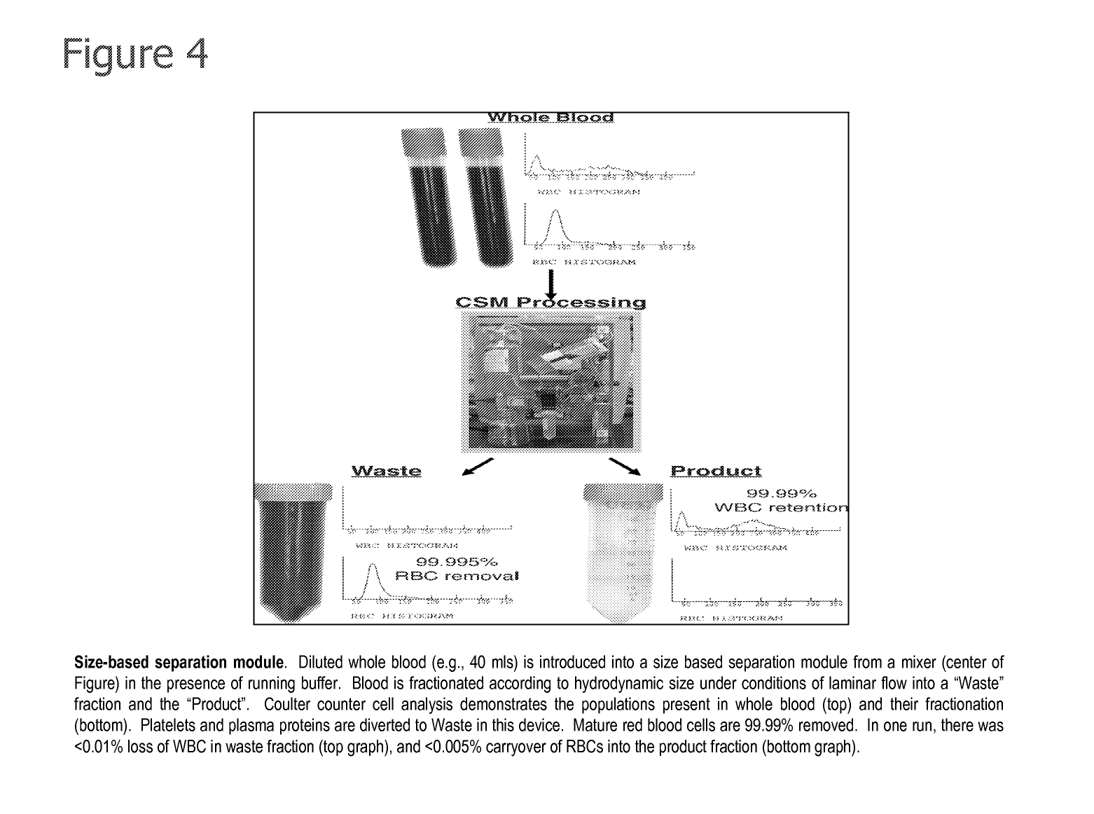

In one embodiment, the CSM comprises an array of gaps of identical dimensions with each row of posts shifted horizontally with respect to the previous row by Δλ, where λ is the center-to-center distance between the posts (FIGS. 2 and 3). The parameter Δλ/λ (the “bifurcation ratio,” ε) determines the ratio of flow bifurcated to the left of the next post. In FIG. 3, ε is ⅓, and the flux through a gap is divided essentially into thirds. In general, if the flux through a gap between two posts is φ, the minor flux is εφ, and the major flux is (1−εφ). Although each of the three fluxes through a gap weaves around the array of posts, the average direction of each flux is in the overall direction of flow. The fluid flow carries cells that are separated through the array of gaps and the flow is aligned at a small angle (flow angle) with respect to a line-of-sight of the array. Cells larger than a critical size migrate along the line-of-sight in the array, whereas those smaller than the critical size follow the flow in a different direction (FIG. 3c). Cells moving with the major flux are transferred sequentially to the major flux passing through each gap. In one embodiment, the device can fractionate maternal blood, thus separating maternal erythrocytes, and platelets (“waste”, FIG. 4) from granulocytes, mononuclear cells, maternal and fetal nucleated red blood cells (“product”, FIG. 4).

A CSM can provide size-based separation (FIG. 3) thus separating a first component of the mixed sample (e.g., fetal cells), which comprises cells or cell components that are larger than a critical size, in a first direction, and a second component of the mixed sample (e.g. enucleated maternal red blood cells), which comprises cells or cell components that are smaller than a critical size, towards a second exit port.

A CSM can comprise one or more array of obstacles, wherein an array can have obstacles of the same or different shape. In various embodiments, obstacles can have shapes including but not limited to diamonds, hexagons, octagons, rectangles, squares, circles, semi-circles, triangles and ellipses. The CSM can comprise one or more arrays of obstacles that form a network of gaps (FIG. 3). Such obstacles can vary in post diameters, spacing, periodicity, width, height to produce varying fluid flow rates and interaction with cells or cellular components present in a sample (FIG. 2). Such array formats can be manufactured using methods familiar to the artisan including but not limited to lasering, micromachining, photolithography, reactive ion etching, ion beam milling, embossing, electroforming, electroplating, injection molding, compression molding, casting, or reaction injection molding. The arrays are comprised in the CSM module (FIG. 4) to process a sample to separate cell populations.

In one embodiment, over 90% of unwanted RBCs are removed while over 90% of WBCs are retained. In various embodiments, over about 80, 85, 90, 91, 92, 93, 94, 95, 96, 97, 98, 99, 99.1, 99.2, 99.3, 99.4, 99.5, 99.6, 99.7, 99.8, 99.9, 99.91, 99.92, 99.93, 99.94, 99.95, 99.96, 99.97, 99.98, 99.99% of RBCs or enucleated RBC's are separated out (e.g., waste). In other embodiments, over about 80, 85, 90, 91, 92, 93, 94, 95, 96, 97, 98, 99, 99.1, 99.2, 99.3, 99.4, 99.5, 99.6, 99.7, 99.8, 99.9, 99.91, 99.92, 99.93, 99.94, 99.95, 99.96, 99.97, 99.98, 99.99% of WBCs and/or nucleated RBC's (including fetal NRBC's) are retained.

Therefore, administering a blood sample to the CSM module can result in high percentage depletion of RBCs and high percentage retention of nucleated cells and high % elimination of plasma proteins.

A blood sample comprising a mixed population of cells can be applied to a microfluidic cell separation module (CSM) to remove red blood cells (RBCs) and plasma proteins, which module comprises one or more array of obstacles. An array can be configured to separate cells smaller than a critical size from those larger than the critical size by adjusting the size of the gaps, obstacles, and offset in the period between each successive row of obstacles. For example, in some embodiments, obstacles and/or gaps between obstacles can be up to 10, 20, 50, 70, 100, 120, 150, 170, or 200 microns in length or about 2, 4, 6, 8 or 10 microns in length. In some embodiments, an array for size-based separation includes more than 100, 500, 1,000, 5,000, 10,000, 50,000 or 100,000 obstacles that are arranged into more than 10, 20, 50, 100, 200, 500, or 1000 rows. Preferably, obstacles in a first row of obstacles are offset from a previous (upstream) row of obstacles by up to 50% the period of the previous row of obstacles. In some embodiments, obstacles in a first row of obstacles are offset from a previous row of obstacles by up to 45, 40, 35, 30, 25, 20, 15 or 10% the period of the previous row of obstacles. Furthermore, the distance between a first row of obstacles and a second row of obstacles can be tip to 10, 20, 50, 70, 100, 120, 150, 170 or 200 microns.

A particular offset can be continuous (repeating for multiple rows) or non-continuous. In some embodiments, a separation module includes multiple discrete arrays of obstacles fluidly coupled such that they are in series with one another. Each array of obstacles has a continuous offset. But each subsequent (downstream) array of obstacles has an offset that is different from the previous (upstream) offset. Preferably, each subsequent array of obstacles has a smaller offset that the previous array of obstacles. This allows for a refinement in the separation process as cells migrate through the array of obstacles. Thus, a plurality of arrays can be fluidly coupled in series or in parallel, (e.g., more than 2, 4, 6, 8, 10, 20, 30, 40, 50). Fluidly coupling separation modules (e.g., arrays) in parallel allows for high-throughput analysis of the sample, such that at least 1, 2, 5, 10, 20, 50, 100, 200, or 500 ml per hour flows through the enrichment modules or at least 1, 5, 10, or 50 million cells per hour are sorted or flow through the device.

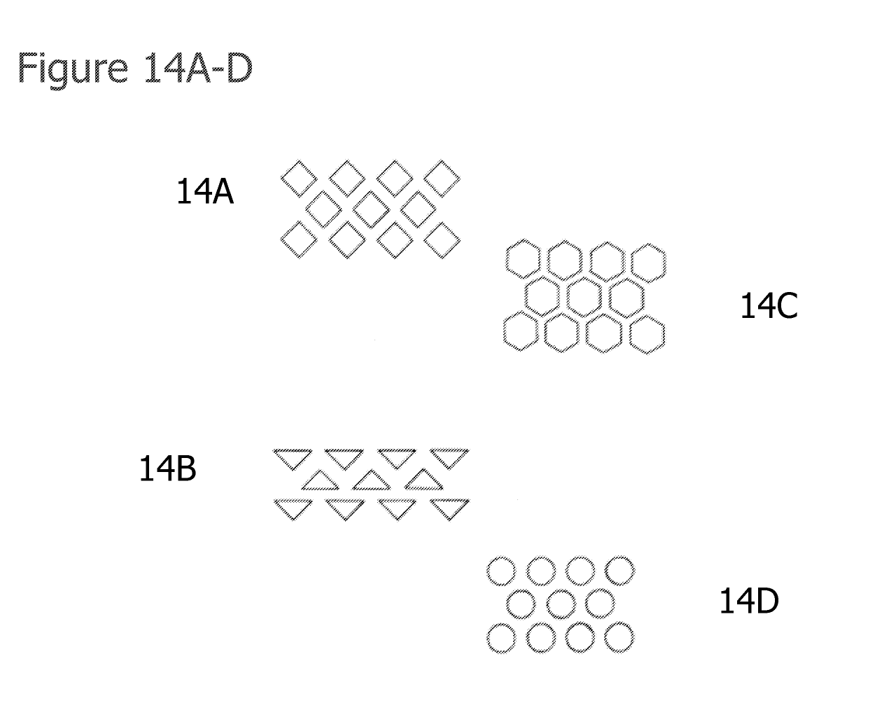

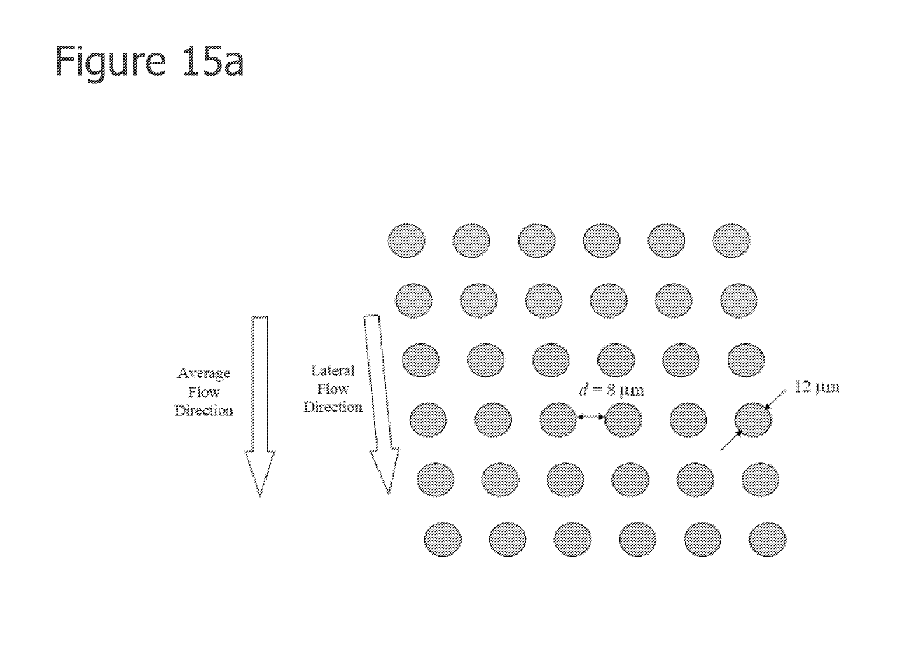

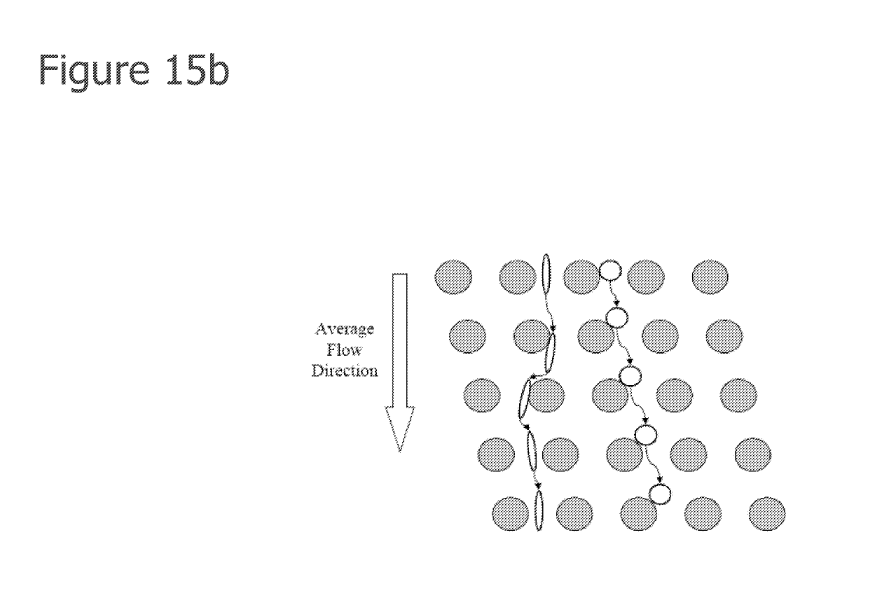

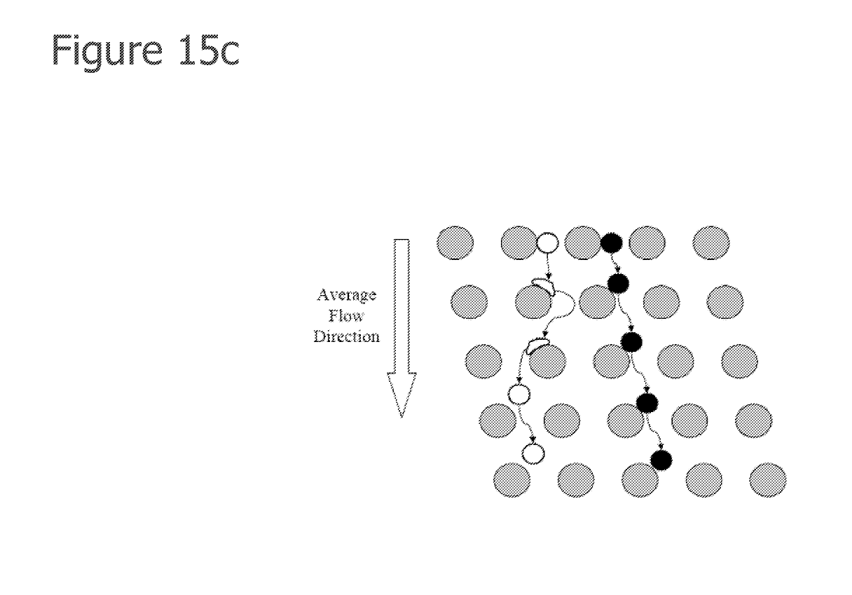

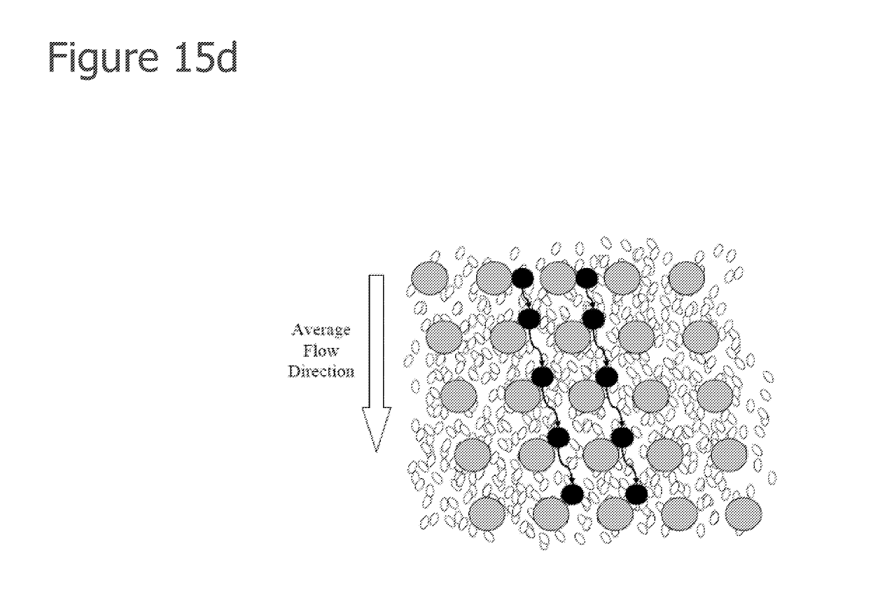

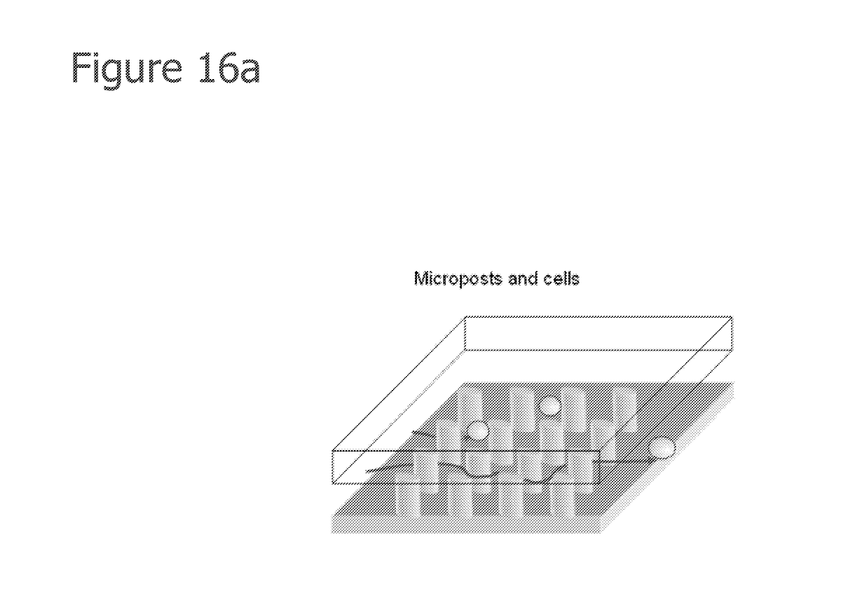

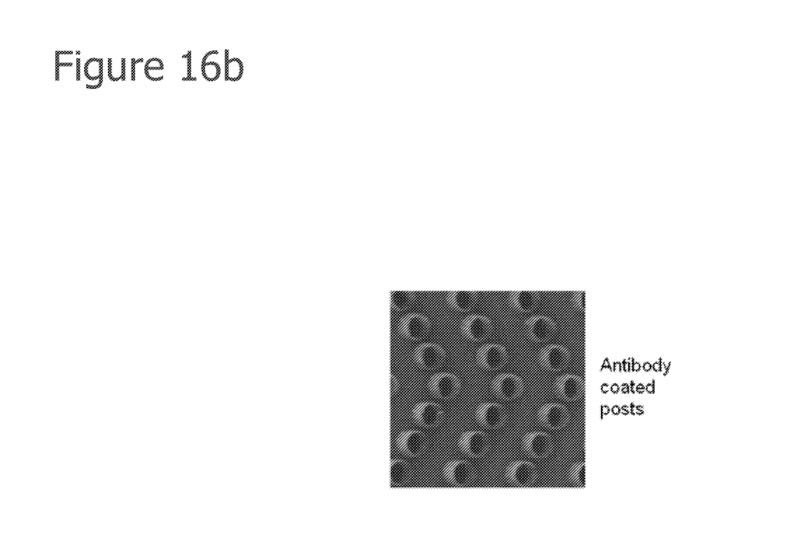

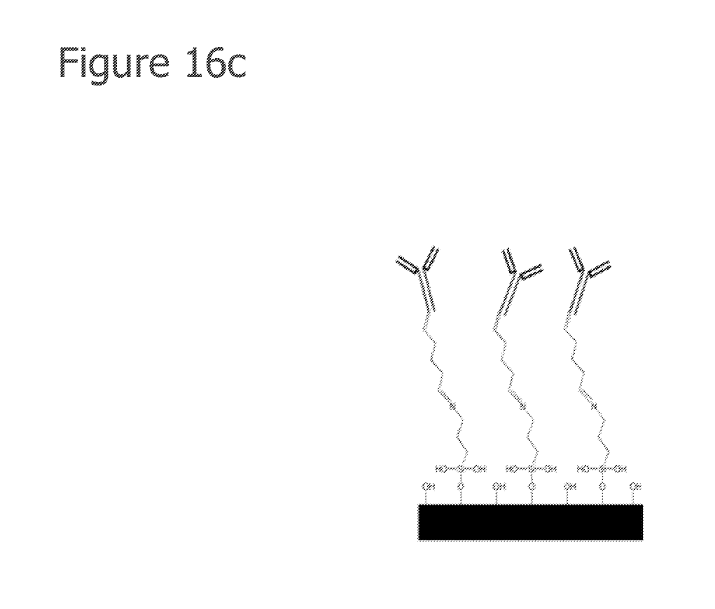

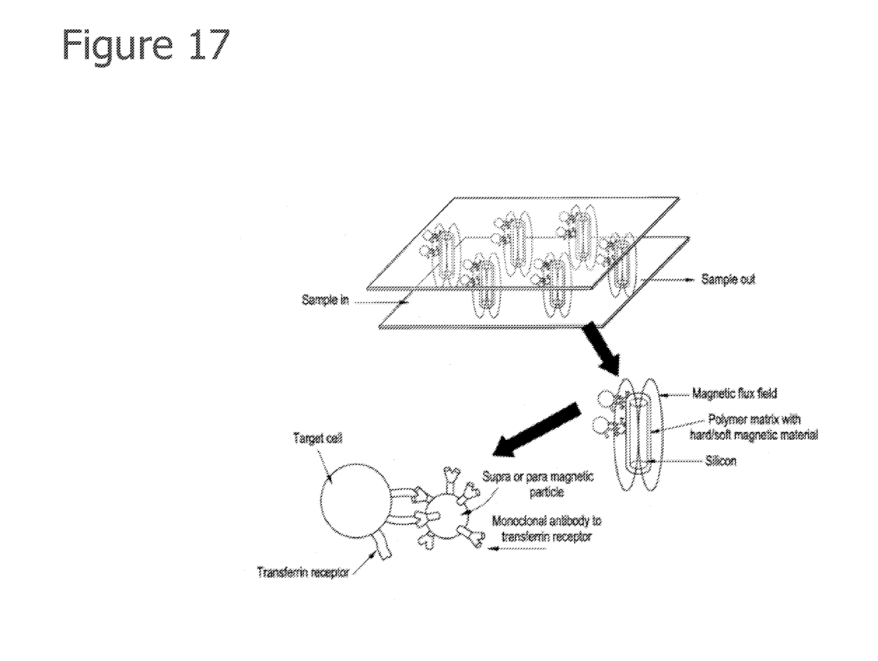

FIGS. 14-16 illustrate examples of size-based separation modules (e.g. a CSM), which can be utilized in methods of the invention. Obstacles (which may be of any shape) are coupled to a flat substrate to form an array of gaps. A transparent cover or lid may be used to cover the array. The obstacles form a two-dimensional array with each successive row shifted horizontally with respect to the previous row of obstacles, where the array of obstacles directs components having a hydrodynamic size smaller than a critical size in a first direction and components having a hydrodynamic size larger that a critical size in a second direction. In some embodiments, the flow of sample into the array of obstacles can be aligned at a small angle (flow angle) with respect to a line-of-sight of the array (lateral flow angle) or perpendicular to lateral flow angle. Optionally, the array is coupled to an infusion pump to perfuse the sample through the obstacles. The flow conditions of the size-based separation module described herein are such that cells are sorted by the array with minimal damage. This allows for downstream analysis of intact cells and intact nuclei to be more efficient and reliable.

In various embodiments, an array can comprise a single or multiple stages. In certain embodiments, the array has maximum pass-through size that is several times larger than the critical size, e.g., when separating white blood cells from red blood cells. Such a result can be obtained by using a combination of large gap size d and small bifurcation ratio ε. In some embodiments, the ε is 1/2, 1/3, 1/10, 1/30, 1/100, 1/200, 1/300 or 1/1000. In such embodiments, obstacles shape may affect the flow profile in the gap; however, the obstacles can be compressed in the flow direction, in order to make the array short. In some embodiments, an array may comprise a bypass channel(s) as described herein.

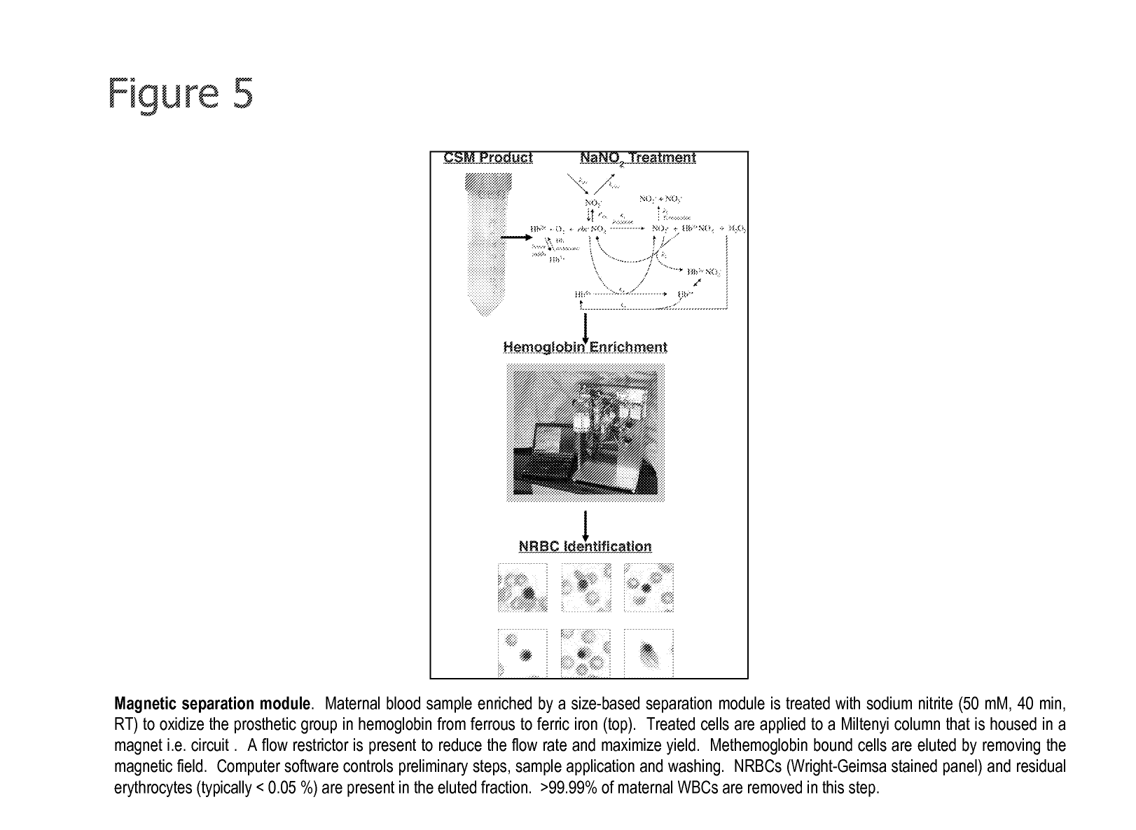

A CSM can comprise a silicon microfluidic chip, a fluidic manifold and controlling instrumentation. In a further embodiment, a silicon chip is housed within a plastic manifold that serves as an interface between the device and the instrument. In other embodiments, components of the instrument include: pneumatic flow control for the blood and buffer supplies, a blood rocking mechanism to ensure sample homogeneity, and an automatic end of run sensor to shut down and clean the system upon completion of sample processing (FIG. 4 or 5).

### Enrichment Techniques

In some embodiments, enrichment that is achieved by size-based separation is followed by, or alternatively preceded by, one or more additional enrichment procedures including magnetic enrichment, antibody enrichment, fluorescence activated cell sorting (FACS), laser microdissection, and magnetic bead separation. In some embodiments, a sample enriched by size-based separation is subjected to affinity/magnetic separation and is further enriched for rare cells using fluorescence activated cell sorting (FACS) or selective lysis of a subset of the cells (e.g. fetal cells). Further separation techniques which can be utilized in the methods of the invention are disclosed in U.S. patent application Ser. Nos. 11/763,245 and 11/763,421. Methods and devices of the invention can be adapted can incorporate enrichment assays and/or genetic analysis such as disclosed in U.S. Pat. Nos. 6,535,293; 6,066,459, 6,913,697; U.S. Patent Application Publication NOs. 20060110783 or 20060160105; international applications: WO91/07660; PCTUS06/012820; 6685841; WO04/29221; WO05/84380; WO05/84374; WO04/37374; WO94/29707; WO01/35071; PCTUS06/021953; PCTUS06/012778; and U.S. application Ser. Nos. 11/051,982; 07/706,393; 07/772,689; 10/560,662; 07/957,736; 11/323,971; 11/229,336; 11/323,962; 60/804,815; 60/804,816; 60/804,817; 60/804,818; Ser. Nos. 11/146,581; 10/248,653; 11/227,469 and 11/071,270.

### Magnetic Enrichment

In one aspect of the invention, an HMEM module functions to capture cells while. In some embodiments, a capture module utilizes a magnetic field to separate and/or enrich one or more analytes (cells) that has a magnetic property or magnetic potential. This magnetic property can be achieved through physical or chemical treatment of the red blood cells. For example, red blood cells which are slightly diamagnetic (repelled by magnetic field) in physiological conditions can be made paramagnetic (attributed by magnetic field) by deoxygenation of the hemoglobin into methemoglobin. Thus, a sample containing one or more red blood cells and one or more non-red blood cells can be enriched for the red blood cells by first inducing a magnetic property and then separating the red blood cells from other analytes using a magnetic field which can be either uniform or non-uniform. For example, a maternal blood sample can flow first through a size-based separation module to remove enucleated cells and cellular components (e.g., analytes having a hydrodynamic size less than 6 μms) based on size. Subsequently, the enriched nucleated cells (e.g., analytes having a hydrodynamic size greater than 6 μms) white blood cells and nucleated red blood cells are treated with a reagent, such as CO₂, N₂ or NaNO₂, that changes the magnetic property of the red blood cells' hemoglobin. The treated sample then flows through a magnetic field (e.g., a column coupled to an external magnet), such that the paramagnetic analytes (e.g., red blood cells) will be captured by the magnetic field while the white blood cells and any other non-red blood cells will flow through the device to result in a sample enriched in nucleated red blood cells (including fnRBC's).

Additional examples of magnetic separation modules are described in U.S. application Ser. No. 11/323,971, filed Dec. 29, 2005 entitled “Devices and Methods for Magnetic Enrichment of Cells and Other Particles” and U.S. application Ser. No. 11/227,904, filed Sep. 15, 2005, entitled “Devices and Methods for Enrichment and Alteration of Cells and Other Particles”.

In some embodiments, one or more methods of the invention herein, further comprise subjecting a sample to a magnetic field. The ability of the CSM to process large blood volumes (>10 mL) with high % RBC removal efficiency enhances separation of RBCs from other cells present in blood, based on the intrinsic magnetic properties of hemoglobin. In one embodiment, a sample is treated so as to convert diamagnetic hemoglobin to paramagnetic methemoglobin, and then capturing the methemoglobin-containing cells with a high gradient magnetic field. This process purifies all or substantially all RBCs away from other blood cells.

In one example, RBCs are treated with sodium nitrite (50 mM, 20 minutes, room temperature), which oxidizes the iron prosthetic group of hemoglobin (and other iron-containing proteins) from ferrous (Fe²⁺) to ferric (Fe³⁺) iron (FIG. 5). The appropriate concentration of sodium nitrite converts virtually all hemoglobin to methemoglobin. Ferric hemoglobin (“methemoglobin”) is paramagnetic and cells containing it can be captured using a magnet. For example, a standard Miltenyi LS column was outfitted with a specially designed magnet to create a Magnetic Hemoglobin Enrichment Module that isolates fNRBCs from the CSM-enriched population of maternal blood. The powerful combination of enriching all nucleated cells from whole blood with the CSM followed by capturing all hemoglobin-containing cells results in a system capable of isolating NRBCs from whole blood with unprecedented sensitivity and specificity (see Table 1).

In various embodiments, the systems herein can include a device for producing a sample enriched in a first cell or component thereof (e.g., fetal NRBC) relative to a second cell or component (e.g., white blood cells) including a channel through which the first cell or component flows; and a magnet that produces a magnetic field of between 0.05 and 5.0 Tesla and a magnetic field gradient of between 100 Tesla/m and 1,000,000 Tesla/m in the channel. The first cell or component may be retained in the channel, and the second component may not be retained in the channel, or vice versa. The channel may include first and second outlets, where the first cell or component thereof is directed into the first outlet, while the second component is directed into the second outlet. The device may also include pump capable of producing a flow rate of greater than 50,000 cells or components thereof flowing into the channel per second. The first cell can be, for example, a nucleated red blood cell, e.g., a fetal nucleated red blood cell, and the second cell can be, for example, an enucleated red blood cell or white blood cell. The first cell includes, for example, fetal hemoglobin, adult hemoglobin, methemoglobin, myoglobin, or a cytochrome.

The systems herein may also include a reservoir containing an oxygenating or deoxygenating agent (e.g., sodium nitrite, CO, CO₂, N₂, or NaNO₂), or other reagent capable of altering a magnetic property of cells present in a sample, coupled to the array of obstacles or the channel. Such agent can optionally oxidize the hemoglobin and increase its magnetic susceptibility. In one embodiment, a sample comprising the first cell or component thereof and second cell or component thereof are first put in contact with the deoxygenating agent. Such deoxygenating agent renders the first cell or cell component thereof paramagnetic (attracted to a magnet, or reactive to a magnetic field).

The passage of the first cell is altered, for example, based on an intrinsic or extrinsic magnetic property. An exemplary magnetic field strength for use in the systems is between 0.5 and 5.0 Tesla, and an exemplary magnetic field gradient is between 100 Tesla/m and 1,000,000 Tesla/m. The systems herein may also include pump capable of producing a flow rate of greater than 50,000 cells or components thereof flowing into the channel per second.

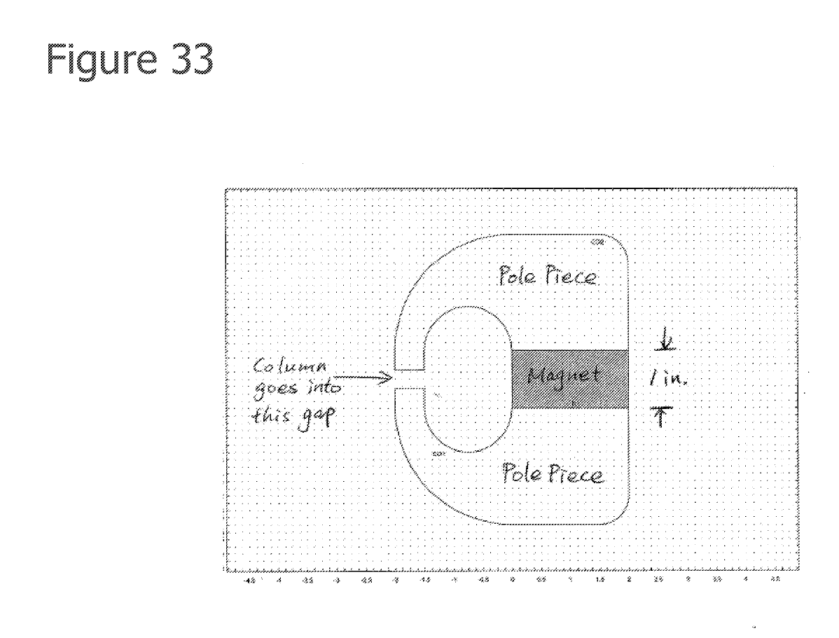

In one embodiment, the magnet is 1.5 inch high, comprised of low carbon steel (pole pieces), and a Neodymium iron boron magnet (NdFeB). The magnet itself is 1″×2″×2″. The magnet (including pole pieces; see FIG. 33) creates a field of 1.4 Tesla across a magnetic column (“LS column” from Miltenyi Biotechnology). The cells flow through the column at a linear velocity of about 0.5 mm/s.

Using the methods herein, nRBCs or nRBC's enriched using size-based separation from maternal blood are treated with sodium nitrite (50 mM, 40 minutes, room temperature), which oxidizes the iron prosthetic group of hemoglobin (and other iron-containing proteins) from ferrous (Fe²⁺) to ferric (Fe³⁺) iron. This concentration of sodium nitrite converts virtually all hemoglobin to methemoglobin. Ferric hemoglobin (“methemoglobin”) is paramagnetic and cells containing it can be captured on a powerful magnet. A standard Miltenyi LS column (a magnetic matrix) is outfitted with a magnet to create a magnetic enrichment module or magnetic hemoglobin enrichment module (MHEM) that isolates fnRBCs from a size-based separation module such as a CSM-enriched population of maternal blood.

Preferably a system herein comprises a size-based separation module fluidly coupled to a magnetic based separation module. The size based separation is configured to remove enucleated red blood cells (RBC) and platelets from a blood sample. The magnetic based separation is configured to enrich remaining cells (e.g., nucleated cells) based on inherent magnetic property. Preferably, a magnetic based separation module removes substantially all or all white blood cells (WBCs) from a sample.

In some cases, when the analyte desired to be separated (e.g., red blood cells or white blood cells) is not ferromagnetic or does not have a magnetic property, a magnetic particle (e.g., a bead) or compound (e.g., Fe³⁺) can be coupled to the analyte to give it a magnetic property. For example, a bead can be coupled to an antibody that selectively binds to an analyte of interest can be decorated with an antibody selected from the group of anti CD71 or CD75. In some embodiments a magnetic compound, such as Fe³⁺, can be couple to an antibody such as those described above. The magnetic particles or magnetic antibodies herein may be coupled to any one or more of the devices herein prior to contact with a sample or may be mixed with the sample prior to delivery of the sample to the device(s).

A magnetic field used to separate analytes/cells in any of the embodiments herein can be uniform or non-uniform as well as external or internal to the device(s) herein. An external magnetic field is one whose source is outside a device herein (e.g., container, channel, obstacles). An internal magnetic field is one whose source is within a device contemplated herein. An example of an internal magnetic field is one where magnetic particles may be attached to obstacles present in the device (or manipulated to create obstacles) to increase surface area for analytes to interact with to increase the likelihood of binding. Analytes captured by a magnetic field can be released by demagnetizing the magnetic regions retaining the magnetic particles. For selective release of analytes from regions, the demagnetization can be limited to selected obstacles or regions. For example, the magnetic field can be designed to be electromagnetic, enabling turn-on and turn-off off the magnetic fields for each individual region or obstacle at will.

The size-based and/or magnetic based enrichment modules, described herein, can be utilized to isolate, select and identify fetal cells or cell components (e.g., nuclei, RNA, DNA, proteins). Such components can subsequently be assayed using for cell-specific differentially expressed biomarkers (e.g., beta and gamma globins) for purposes, including but not limited to diagnoses of a disease or condition.

### Antibody Enrichment

In some embodiments, size based separation is followed by affinity separation. One example of an affinity-based separation module is an array of obstacles that are adapted for complete sample flow through, but for the fact that the obstacles are covered with binding moieties that selectively bind one or more analytes (e.g., cell population) of interest (e.g., red blood cells, fetal cells, or nucleated cells) or analytes not-of-interest (e.g., white blood cells). (See FIG. 2) Binding moieties can include e.g., proteins (e.g., ligands/receptors), nucleic acids having complementary counterparts in retained analytes, antibodies, etc. In some embodiments, an affinity-based separation module comprises a two-dimensional array of obstacles covered with one or more antibodies selected from the group consisting of: anti-CD71, anti-CD235a, anti-CD36, anti-carbohydrates, anti-selectin, anti-CD 34, anti-CD45, anti-GPA, anti-CD, anti-7HbF or anti-HAE9 and anti-antigen-i.

In some embodiments, a sample enriched by size-based separation is also enriched for rare cells such as fetal cells using fluorescence activated cell sorting (FACS) or selective lysis of a subset of the cells (e.g. fetal cells or non-fetal cells).

In some embodiment, fetal cells or fetal DNA is distinguished from non-fetal cells or non-fetal DNA by forcing the rare cells (fetal cells) to become apoptotic, thus condensing their nuclei and optionally ejecting their nuclei. Rare cells such as fetal cells can be forced into apoptosis using various means including subjecting the cells to hyperbaric pressure (e.g. 4% CO₂). The condensed nuclei can then be detected and/or isolated for further analysis using any technique known in the art including but not limited to, DNA gel electrophoresis, in situ labeling of DNA nicks (terminal deoxynucleotidyl transferase (TdT))-mediated dUTP in situ nick labeling (also known as TUNEL) (Gavrieli, Y., et al. J. Cell Biol 119:493-501 (1992)) and ligation of DNA strand breaks having one or two-base 3′ overhangs (Taq polymerase-based in situ ligation). (Didenko V., et al. J. Cell Biol. 135:1369-76 (1996)). Other fetal cell enrichment methods can be adapted for use with one or more embodiments of the present invention such as enrichment methods disclosed in U.S. Pat. Nos. 5,714,325; 5,646,004; 5,580,724; 5,714,325; 5,580,724; U.S. Patent Application Publications: 20050049793, 20050164241, and Ser. Nos. 11/763,245 and 11/763,421; or international application WO-A-97/42503.

### The Enrichment Product

In any of the embodiments herein, the enrichment steps performed have a specificity and/or sensitivity ≧50, 60, 70, 80, 90, 95, 96, 97, 98, 99, 99.1, 99.2, 99.3, 99.4, 99.5, 99.6, 99.7, 99.8, 99.9 or 99.95% The retention rate of the enrichment module(s) herein is such that ≧50, 60, 70, 80, 90, 91, 92, 93, 94, 95, 96, 97, 98, 99, or 99.9% of the analytes or cells of interest (e.g., nucleated cells or nucleated red blood cells or nucleated from red blood cells) are retained. Simultaneously, the enrichment modules are configured to remove ≧50, 60, 70, 80, 85, 90, 91, 92, 93, 94, 95, 96, 97, 98, 99, or 99.9% of all unwanted analytes (e.g., red blood-platelet enriched cells) from a sample.

Any or all of the enrichment steps can occur with minimal dilution of the sample. For example, in some embodiments the analytes of interest are retained in an enriched solution that is less than 50, 40, 30, 20, 10, 9.0, 8.0, 7.0, 6.0, 5.0, 4.5, 4.0, 3.5, 3.0, 2.5, 2.0, 1.5, 1.0, or 0.5 fold diluted from the original sample. In some embodiments, any or all of the enrichment steps increase the concentration of the analyte of interest (fetal cell), for example, by transferring them from the fluid sample to an enriched fluid sample (sometimes in a new fluid medium, such as a buffer). The new concentration of the analyte of interest may be at least 2, 4, 6, 8, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, 1,000,000,000, 2,000,000,000, or 5,000,000,000 fold more concentrated than in the original sample. For example, a 10 times concentration increase of a first cell type out of a blood sample means that the ratio of first cell type/all cells in a sample is 10 times greater after the sample was applied to the apparatus herein. Such concentration can take a fluid sample (e.g., a blood sample) of greater than 10, 15, 20, 50, or 100 mL total volume comprising rare components of interest, and it can concentrate such rare component of interest into a concentrated solution of less than 0.5, 1, 2, 3, 5, or 10 mL total volume.

The final concentration of fetal cells in relation to non-fetal cells after enrichment can be about 1/10,000-1/10, or 1/1,000-1/100. In some embodiments, the concentration of fetal cells to maternal cells may be up to 1/1,000, 1/100, or 1/10 or as low as 1/100, 1/1,000 or 1/10,000.

Thus, detection and analysis of the fetal cells can occur even if the non-fetal (e.g. maternal) cells are >50%, 60%, 70%, 80%, 90%, 95%, or 99% of all cells in a sample. In some embodiments, fetal cells are at a concentration of less than 1:2, 1:4, 1:10, 1:50, 1:100, 1:1000, 1:10,000, 1:100,000, 1,000,000, 1:10,000,000 or 1:100,000,000 of all cells in a mixed sample to be analyzed or at a concentration of less than 1×10⁻³, 1×10⁻⁴, 1×10⁻⁵, 1×10⁻⁶, or 1×10⁻⁶ cells/μL of the mixed sample. Over all, the number of fetal cells in a mixed sample, (e.g. enriched sample) has up to 1, 2, 3, 4, 5, 6, 7, 8, 9, 10, 15, 20, 30, 40, 50, 100 total fetal cells. Such analysis methods are also disclosed in U.S. patent application Ser. Nos. 11/763,245 and 11/763,421, which are incorporated herein by reference in their entirety.

Binning

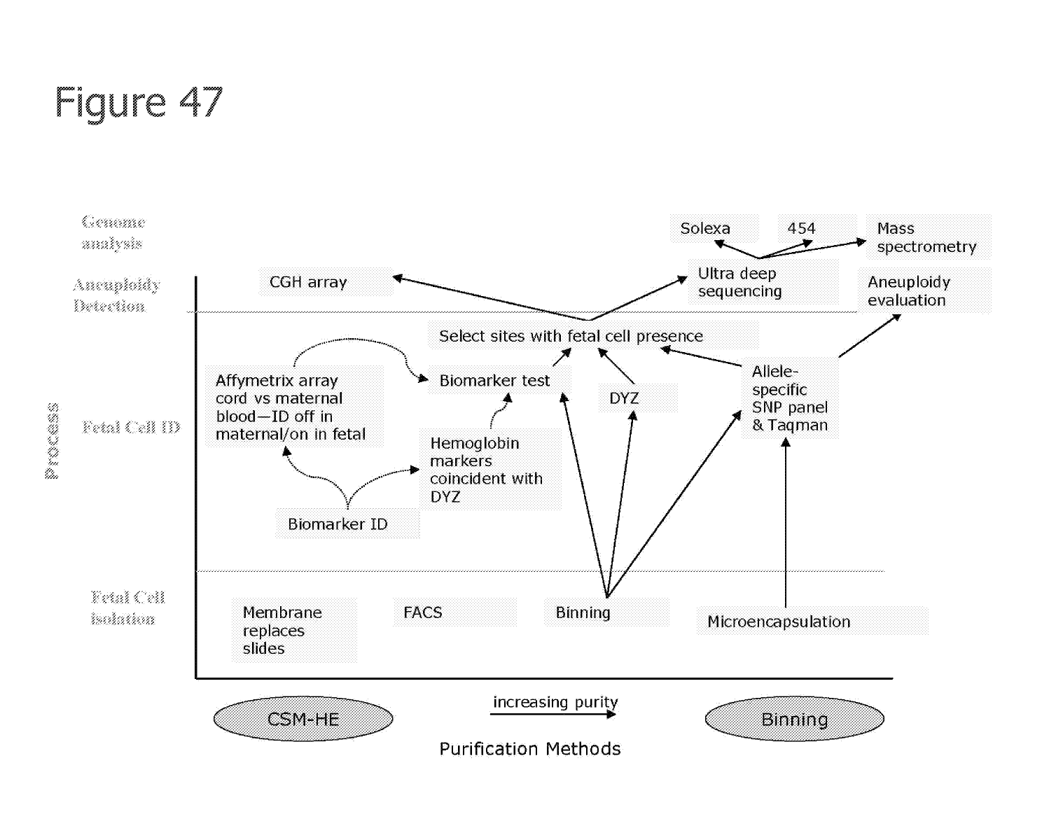

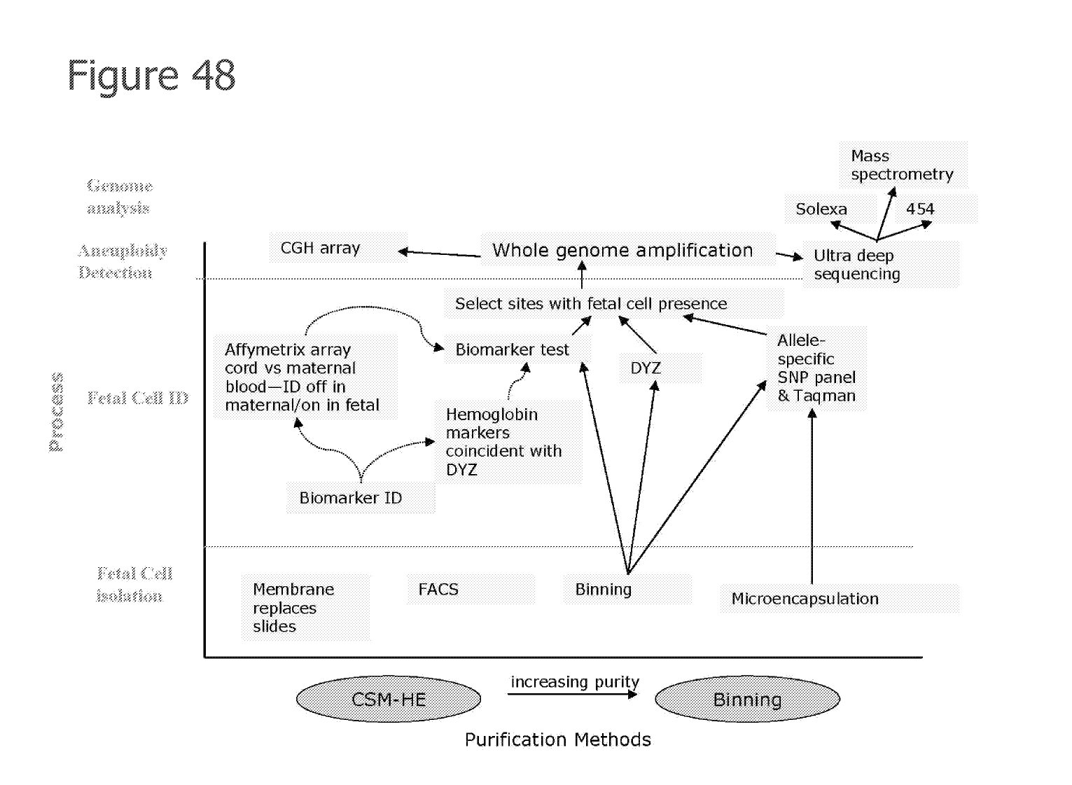

Furthermore, in some embodiments, enriched target cells (e.g., fnRBC) can be “binned” prior to further analysis. (FIGS. 47 & 48). Binning is any process which results in the reduction of complexity and/or total cell number of the enriched cell output. Binning may be performed by any method known in the art or described herein. One method of binning the enriched cells is by serial dilution. Such dilution may be carried out using any appropriate platform (e.g., PCR wells, microtiter plates). Other methods include nanofluidic systems which separate samples into droplets (e.g., BioTrove, Raindance, Fluidigm). Such nanofluidic systems may result in the presence of a single cell present in a nanodroplet.

Binning may be preceded by positive selection for target cells including, but not limited to affinity binding (e.g. using anti-CD71 antibodies). Alternately, negative selection of non-target cells may precede binning. For example, output from the size-based separation module may be passed through a magnetic hemoglobin enrichment module (MHEM) which selectively removes WBCs from the enriched sample.

For example, the possible cellular content of output from enriched maternal blood which has been passed through a size-based separation module (with or without further enrichment by passing the enriched sample through a MHEM) may consist of: 1) approximately 20 fnRBC; 2) 1,500 mnRBC; 3) 4,000-40,000 WBC; 4) 15×10⁶ RBC. If this sample is separated into 100 bins (PCR wells or other acceptable binning platform), each bin would be expected to contain: 1) 80 negative bins and 20 bins positive for one fnRBC; 2) 150 mnRBC; 3) 400-4,000 WBC; 4) 15×10⁴ RBC. If separated into 10,000 bins, each bin would be expected to contain: 1) 9,980 negative bins and 20 bins positive for one fnRBC; 2) 8,500 negative bins and 1,500 bins positive for one mnRBC; 3) <1-4 WBC; 4) 15×10² RBC. One of skill in the art will recognize that the number of bins may be increased depending on experimental design and/or the platform used for binning. The reduced complexity of the binned cell populations may facilitate further genetic and cellular analysis of the target cells.

Analysis may be performed on individual bins to confirm the presence of target cells (e.g. fnRBC) in the individual bin. Such analysis may consist of any method known in the art, including, but not limited to, FISH, PCR, STR detection, SNP analysis, biomarker detection, and sequence analysis (FIGS. 47 & 48).

### Analysis of Enrichment Product

### Lysis/Permeabilization

In some embodiments, a CSM and/or MHEM output comprising the cells of interest (e.g., fnRBCs) is lysed to isolate nuclei using cell lysis techniques known in the art, such as methods and compositions as disclosed in WO 2004/029221, WO 2004/113877 or U.S. Pat. No. 5,837,115. For example, cells can be permeabilized by treating them with a cytoskeletal buffer (CB buffer: 100 mM NaCl, 30 mM sucrose, 3 mM MgCl₂, 10 mM PIPES, pH 6.8), 30 s (CB buffer can be replaced with 1×PBS).

In other embodiments, a blood sample can be combined with an agent that selectively lyses one or more cells or components in a blood sample. For example, cells can undergo selective lysis thus releasing their nuclei when a blood sample including fetal cells is combined with de-ionized water. Such selective lysis allows for the subsequent enrichment of nuclei using, e.g., size or affinity based separation. In another example platelets and/or enucleated red blood cells are selectively lysed to generate a sample enriched in nucleated cells, such as fetal nucleated red blood cells (fnRBC) and maternal nucleated blood cells (mnBC). The fnRBC's can subsequently be separated from the mnRBC's using, e.g., affinity to antigen-i or magnetism differences in fetal and adult hemoglobin.

### Arraying

Enriched cells or cellular components (e.g., nuclei) may be transferred to a solid support for further analysis. For example, in some embodiments, enriched product of cells or cellular components (e.g., sample comprising fetal cells or fetal nuclei) are cytospun onto a slide. In such cases anywhere from 10²-10⁷ nuclei can be applied to one slide.

### Marker Genes

In some embodiments, biomarkers are utilized to determine that a cell is of fetal origin. For example, either after enrichment or after detection of fetal abnormality or lack thereof, fetal biomarkers can be used to detect and/or isolate fetal cells. This is accomplished by distinguishing between fetal and maternal nRBCs based on relative expression of a gene (e.g., DYS1, SRY, DYZ, CD-71, ε-, ζ-, γ- and α-globins) that is differentially expressed during fetal development. In preferred embodiments, biomarker genes are differentially expressed in the first and/or second trimester.

Yolk sac erythroblasts synthesize ε-, ζ-, γ- and α-globins, which combine to form the embryonic hemoglobins. Between six and eight weeks, the primary site of erythropoiesis shifts from the yolk sac to the liver, the three embryonic hemoglobins are replaced by fetal hemoglobin (HbF) as the predominant oxygen transport system, and ε- and ζ-globin production gives way to γ-, α- and β-globin production within definitive erythrocytes (Peschle et al., 1985). HbF remains the principal hemoglobin until birth, when the second globin switch occurs and β-globin production accelerates. Hemoglobin (Hb) is a heterodimer composed of two identical a globin chains and two copies of a second globin.

“Differentially expressed,” as applied to nucleotide sequences or polypeptide sequences in a cell or cell nuclei, refers to differences in over/under-expression of that sequence when compared to the level of expression of the same sequence in another sample, a control or a reference sample. In some embodiments, expression differences can be temporal and/or cell-specific. For example, for cell-specific expression of biomarkers, differential expression of one or more biomarkers in the cell(s) of interest can be higher or lower relative to background cell populations. Detection of such difference in expression of the biomarker may indicate the presence of a rare cell (e.g., fnRBC) versus other cells in a mixed sample (e.g., background cell populations). In other embodiments, a ratio of two or more such biomarkers that are differentially expressed can be measured and used to detect rare cells. Therefore, a ratio of two or more markers provides an index that corresponds to whether a fetal or maternal cell or cell component is present. In other embodiments, such an index is comprised of at least one marker to a gene that is not differentially expressed as between a fetus or adult.

Therefore, in various embodiments, one or more marker genes used to detect fetal cells include but are not limited to a Y-chromosome specific gene, DYS1, SRY, DYZ, CD-71, ε-globin, γ-globin, ζ-globin, α-globin and β-globin or a combination thereof. In various embodiments, combinations of such markers measured include but are not limited to two or more globins, DYS1 and one or more globins, DYZ and one or more globins. SRY and one or more globins.

In one embodiment, fetal biomarkers detected comprise differentially expressed hemoglobins (e.g., ε-glohin, γ-globin and β-globin). Erythroblasts (nRBCs) are very abundant in the early fetal circulation, virtually absent in normal adult blood and by having a short finite lifespan, there is no risk of obtaining fnRBC which may persist from a previous pregnancy. Furthermore, unlike trophoblast cells, fetal erythroblasts are not prone to mosaic characteristics.

Due to differential gene expression during fetal development, the composition of the second chain changes from ε globin during early embryonic development (1 to 4 weeks of gestation) to γ globin during fetal development (6 to 8 weeks of gestation) to β-globin in neonates and adults as illustrated in (Table 1).

TABLE 1
Relative expression of ε, γ and β in maternal and fetal RBCs.

| | | ε | γ | B |
|---|---|---|---|---|
| 1^{st} trimester | Fetal | ++ | ++ | − |
| | Maternal | − | +/− | ++ |
| 2^{nd} trimester | Fetal | − | ++ | +/− |
| | Maternal | − | +/− | ++ |

In the late-first trimester, the earliest time that fetal cells may be sampled by CVS, fnRBCs contain, in addition to a globin, primarily ε and γ globin. In the early to mid second trimester, when amniocentesis is typically performed, fnRBCs contain primarily γ globin with some adult β globin. Maternal cells contain almost exclusively α and β globin, with traces of γ detectable in some samples. Therefore, by measuring the relative expression of the ε, γ and β genes in RBCs purified from maternal blood samples, the presence of fetal cells in the sample can be determined. Furthermore, positive controls can be utilized to assess failure of the FISH analysis itself.

In various embodiments, fetal cells are distinguished from maternal cells based on the differential expression of hemoglobins β, γ or ε. Expression levels or RNA levels can be determined in the cytoplasm or in the nucleus of cells. Thus in some embodiments, the methods herein involve determining levels of messenger RNA (mRNA), ribosomal RNA (rRNA), or nuclear RNA (nRNA). Therefore, in one embodiment, relative expression of ε, γ and β globins is used to identity one or more fetal RBCs. Furthermore, in some embodiments in determining whether an enriched cell is a fetal or maternal cell a ratio measured is of γ/β, γ+ε/β, β+ε/γ or β/γ.

In some embodiments, identification of fnRBCs can be achieved by measuring the levels of at least two hemoglobins in the cytoplasm or nucleus of a cell. In various embodiments, identification and assay is from 1, 2, 3, 4, 5, 6, 7, 8, 9, 10, 15 or 20 fetal nuclei. Furthermore, total nuclei arrayed on one or more slides can number from about 100, 200, 300, 400, 500, 700, 800, 5000, 10,000, 100,000, 1,000,000, 2,000,000 to about 3,000,000. In some embodiments, a ratio for γ/β or ε/β is used to determine the presence of fetal cells, where a number less than one indicates that a fnRBC(s) is not present. In some embodiments, the relative expression of γ/β or ε/β provides a fnRBC index (“FNT”), as measured by γ or ε relative to β. In some embodiments, a FNI for γ/β greater than 5, 10, 15, 20, 25, 30, 35, 40, 45, 90, 180, 360, 720, 975, 1020, 1024, 1250 to about 1250, indicate that a fnRBC(s) is present. In yet other embodiments, a FNI for γ/β of less than about 1 indicates that a fnRBC(s) is not present. Preferably, the above FNI is determined from a sample obtained during a first trimester. However, similar ratios can be used during second trimester and third trimester.

In some embodiments, the expression levels are determined by measuring nuclear RNA transcripts including, nascent or unprocessed transcripts (e.g., RNA FISH or RT-PCR). In another embodiment, expression levels are determined by measuring mRNA, including ribosomal RNA. There are many methods known in the art for imaging (e.g., measuring) nucleic acids or RNA including, but not limited to, using expression arrays from Affymetrix, Inc. or Illumina, Inc. In yet further embodiments, measurements of marker genes comprises measuring protein levels using standard biochemical and immunochemical methods known in the art (e.g., BioRad, antibody immunochemistry).

FIG. 7 illustrates differential expression of gamma versus beta globin in cells enriched using devices and methods of the invention described herein. As provided in FIGS. 7A and 7B the ratio of γ/β globins for maternal cells is substantially distinct from fetal cells isolated during the same time period—FIG. 7C first trimester and FIG. 7D second trimester. As evidenced from the FIGS. 7C and 7D, in fetal cells γ globin levels are higher relative to β globin levels.

Furthermore, as illustrated in FIG. 8 the differential expression provides similar results from relatively small number of cells (e.g., 40 nuclei) FIG. 8A down to a single cell FIG. 8C. Therefore, in some embodiments, the number of fetal cells identified using methods and devices of the invention comprise from 1 to about 200 cells, about 5 to about 50 cells, about 25 to about 100 cells, about 75 to about 200 cells, about 100 to about 500 cells. In various embodiments, the number of fetal cells identified is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 cells.

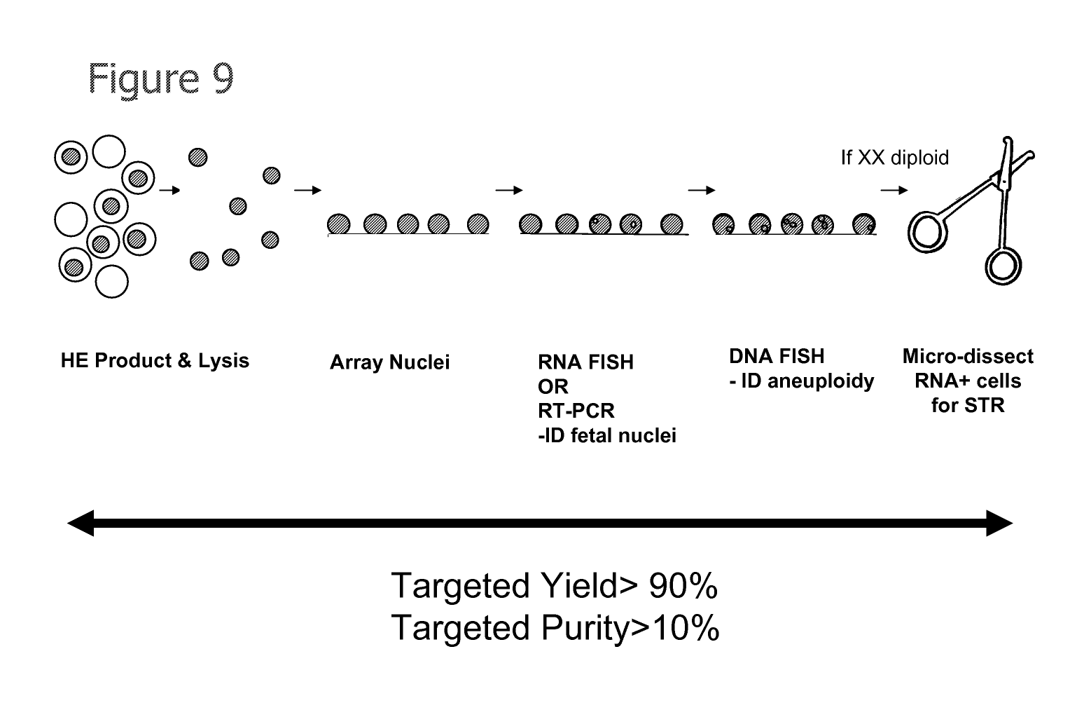

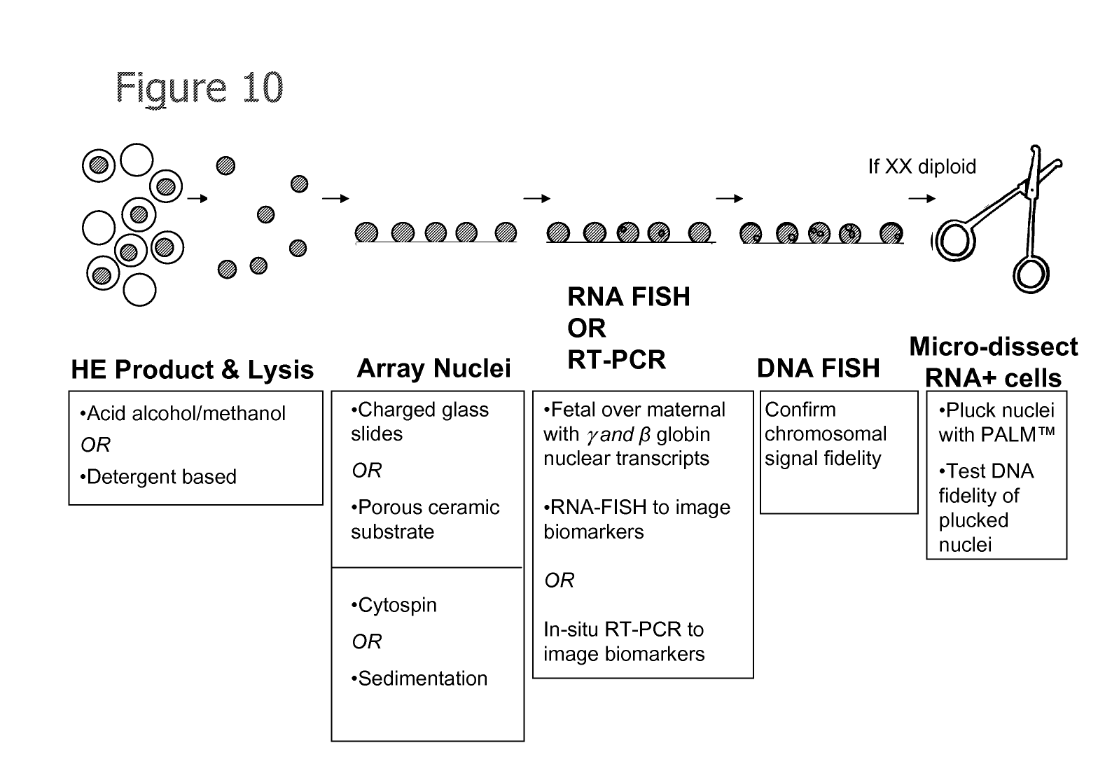

As illustrated in FIG. 9, in one embodiment, marker gene expression is utilized to identify fetal cell(s) (e.g., RNA FISH or RT-PCR), which are screened for an abnormality (e.g., DNA FISH to identify aneuploidy; see also, FIG. 10) and microdissected for STR analysis. Cells enriched through HE can be subjected to lysis through detergent based or acid alcohol/methanol treatment FIG. 10. Furthermore, cells can be arrayed utilizing charged glass slides, porous ceramic substrate and/or cytospin or sedimentation. Fetal cells identified through biomarker imaging, and assayed for chromosomal signal fidelity can be microdissected using PALM to pluck nuclei, which can also be assayed for fidelity FIG. 10.

RT-PCR primers can be designed by targeting the globin variable regions, selecting the amplicon size, and adjusting the primers annealing temperature to achieve equal PCR amplification efficiency. Thus TaqMan probes can be designed for each of the amplicons with well-separated fluorescent dyes, Alexa fluor-355 for ε, Alexa Fluor-488 for γ, and Alexa Fluor-555 for β. The specificity of these primers can be first verified using ε, γ, and β cDNA as templates. The primer sets that give the best specificity can be selected for further assay development. As an alternative, the primers can be selected from two exons spanning an intron sequence to amplify only the mRNA to eliminate the genomic DNA contamination.

The primers selected can be tested first in a duplex format to verify their specificity, limit of detection, and amplification efficiency using target cDNA templates. The best combinations of primers can be further tested in a triplex format for its amplification efficiency, detection dynamic range, and limit of detection.

Various commercially available reagents are available for RT-PCR, such as One-step RT-PCR reagents, including Qiagen One-Step RT-PCR Kit and Applied Biosytems TaqMan One-Step RT-PCR Master Mix Reagents kit. Such reagents can be used to establish the expression ratio of ε, γ and β using purified RNA from enriched samples. Forward primers can be labeled for each of the targets, using Alexa fluor-355 for ε, Alexa fluor-488 for γ, and Alexa fluor-555 for β. Enriched cells can be deposited by cytospinning onto glass slides. Additionally, cytospinning the enriched cells can be performed after in situ RT-PCR. Thereafter, the presence of the fluorescent-labeled amplicons can be visualized by fluorescence microscopy. The reverse transcription time and PCR cycles can be optimized to maximize the amplicon signal:background ratio to have maximal separation of fetal over maternal signature. Preferably, signal background ratio is greater than 5, 10, 50 or 100 and the overall cell loss during the process is less than 50, 10 or 5%.

### Detection of Fetal Abnormality

Fetal abnormality can be detected in a sample enriched for fetal cells using any method known in the art or disclosed herein.

In some cases, fetal abnormality is fetal aneuploidy. Examples of fetal aneuploidy include, but are not limited to, trisomy 13, trisomy 18, trisomy 21 (Down Syndrome), Klinefelter Syndrome (XXY) and other irregular number of sex or autosomal chromosomes. In some embodiments, the fetal abnormality detected is a segmental aneuploidy. Examples of segmental aneuploidy include, but are not limited to, 1p36 duplication, dup(17)(p11.2p11.2) syndrome, Pelizaeus-Merzbacher disease, dup(22)(q11.2q11.2) syndrome, Cat eye syndrome. In some embodiment, the fetal abnormality detected is due to one or more deletions in sex or autosomal chromosomes, including Cri-du-chat syndrome, Wolf-Hirschhorn, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, Steroid sulfatase deficiency, Kallmann syndrome, Microphthalmia with linear skin defects, Adrenal hypoplasia, Glycerol kinase deficiency, Pelizaeus-Merzbacher disease, Testis-determining factor on Y, Azospermia (factor a), Azospermia (factor b), Azospermia (factor c) and 1p36 deletion. In some embodiments, the fetal abnormality to be detected is due to decrease chromosomal number such as XO syndrome.

Fetal abnormalities such as aneuploidy can be detected using DNA FISH to identify an irregular chromosome number. In some cases, DNA FISH probes such those available by Vysis probes (Abbott Laboratories, Downer's Grove, Ill.) are utilized. In one embodiment, FISH probes are administered concurrently to both stain for fetal cells (e.g., marker genes described herein) and to detect one or more abnormalities described herein (e.g., trisomy), or determining paternal versus maternal traits.

In one aspect of the invention, fetal cell detection utilizing the compositions and methods of the invention further comprises using multiple FISH probes specific for the same chromosome. Thus in some embodiments, two Y-chromosome probes are utilized in FISH (i.e., YY-FISH) analysis of fetal cells present in a blood sample. Some embodiments of the invention are directed to methods utilizing XY- and YY-FISH probes. In one embodiment, two different Y chromosome-specific FISH probes (e.g., alpha satellite and classical satellite III regions) are utilized in combination with an X chromosome specific FISH probe. For examples male fetal cells can be detected in maternal blood samples.

In other embodiments, two different FISH probes are used against chromosome 13, 18, 21 or X. In yet further embodiments, two or more different FISH probes are utilized against any chromosome as desired.

In some embodiments, YY-FISH can be conducted with additional staining methods to concurrently, provide further genetic analysis of the cells determined to be of fetal origin (e.g., determining trisomy, aneuploidy etc.). In some other embodiments multiple FISH probes specific for the same chromosome (such as but not limited to regions on chromosome 13, 18, 21 and X) can be used to increase the accuracy of detection of aneuploidy or segmental aneuploidy.

Fetal abnormalities can also be detected using comparative genomics hybridization (CGH) arrays. Comparative Genomic Hybridization (CGH) employs the kinetics of in situ hybridization to compare the copy numbers of different DNA or RNA sequences from a sample, or the copy numbers of different DNA or RNA sequences in one sample to the copy numbers of the substantially identical sequences in another sample. In many useful applications of CGH, the DNA or RNA is isolated from a subject cell or cell population. The comparisons can be qualitative or quantitative. Procedures are described that permit determination of the absolute copy numbers of DNA sequences throughout the genome of a cell or cell population if the absolute copy number is known or determined for one or several sequences. The different sequences are discriminated from each other by the different locations of their binding sites when hybridized to a reference genome, usually metaphase chromosomes but in certain cases interphase nuclei. The copy number information originates from comparisons of the intensities of the hybridization signals among the different locations on the reference genome. The methods, techniques and applications of CGH are known, such as described in U.S. Pat. No. 6,335,167, and in U.S. App. Ser. No. 60/804,818, the relevant parts of which are herein incorporated by reference.

Array CGH has been implemented using a wide variety of techniques. The initial approaches used an arrays produced from large-insert genomic clones such as bacterial artificial chromosomes (BACs). Producing sufficient BAC DNA of adequate purity to make arrays is arduous, so several techniques to amplify small amounts of starting material have been employed. These techniques include ligation-mediated polymerase chain reaction (PCR) (Snijders et al, Nat. Genet. 29:263-64), degenerate primer PCR using one or several sets of primers, and rolling circle amplification. BAC arrays that provide complete genome tiling paths are also available. Arrays made from less complex nucleic acids such as cDNAs, selected PCR products, and oligonucleotides can also be used. Although most CGH procedures employ hybridization with total genomic DNA, it is possible to use reduced complexity representations of the genome produced by PCR techniques. Computational analysis of the genome sequence can be used to design array elements complementary to the sequences contained in the representation. Various single nucleotide polymorphism (SNP) genotyping platforms, some of which use reduced complexity genomic representations, are useful for their ability to determine both DNA copy number and allelic content across the genome.

The different basic approaches to array CGH provide different levels of performance, so some are more suitable for particular applications than others. The factors that determine the performance requirements include the magnitudes of the copy number changes, their genomic extents, the state and composition of the specimen, how much material is available for analysis, and how the results of the analysis will be used. Many applications require reliable detection of copy number changes of much less than 50%, a more stringent requirement than for other microarray technologies. Note that technical details are extremely important and different implementations of the “same” array CGH approach may yield different levels of performance. Various CGH methods are known in the art and are equally applicable to one or more methods of the present invention. For example, COP methods are disclosed in U.S. Pat. Nos. 7,034,144; 7,030,231; 7,011,949; 7,014,997; 6,977,148; 6,951,761; and 6,916,621, the disclosure from each of which is incorporated by reference herein in its entirety.

Fetal abnormalities can also be detected using short tandem repeats (STRs) or variable number of tandem repeats (VNTRs). STRs are short, tandemly repeated DNA sequences which are interspersed throughout the human genome at up to several hundred thousand loci (Koreth, et al. J Pathol. 1996 March; 178(3):239-48). They are also found in animals and plants where they are similarly useful as genetic markers (Orti, et al., Mol Ecol. 1997 June; 6(6):575-80). STRs are typically 2-7 base pairs in length. These loci are highly polymorphic with respect to the number of repeat units they contain and may vary in internal structure as well. Variation in the number of STR repeat units at a particular locus causes the length of the DNA at that locus to vary from allele to allele and from individual to individual. Thus, many allelic variants exist within the human population, and STRs provide a rich source of genetic markers. For example, in one embodiment a blood sample is subjected to a CSM and HE modules described herein to produce an enriched sample of cells, which are micromanipulated such as by microdissection/nucleic isolation, and genetic analysis such as by STR. In a further embodiment STR analysis is applied in an assay of a single cell from a sample (e.g., blood sample). Examples of STRs that can be used to distinguish between maternal and fetal cell(s) include but are not limited to D21S11, D21S1411, D2181412, and D8SS535. In one embodiment, such analysis is applied to a single cell isolated using the compositions and methods of the invention.

STR polymorphic loci selected for analysis can be used to detect non-maternal fetal alleles in the mixed sample and to determine the copy number of such alleles. When amplifying more than one polymorphic loci or DNA regions, primers are selected to be multiplexable (fairly uniform melting temperature, absence of cross-priming on the human genome, and absence of primer-primer interaction based on sequence analysis) with other primer pairs. Primers and Loci are chosen so that the amplicon lengths from a given locus do not overlap with those from another locus.

Amplified STR loci from both mixed (maternal and fetal sample) and, optionally, reference (maternal only) samples, can be quantified for characterization/diagnosis of fetal abnormality using any method known in the art. Examples of such methods include, but are not limited to, gas chromatography, supercritical fluid chromatography, liquid chromatography, including partition chromatography, adsorption chromatography, ion exchange chromatography, size-exclusion chromatography, thin-layer chromatography, and affinity chromatography, electrophoresis, including capillary electrophoresis, capillary zone electrophoresis, capillary isoelectric focusing, capillary electrochromatography, micellar electrokinetic capillary chromatography, isotachophoresis, transient isotachophoresis and capillary gel electrophoresis, comparative genomic hybridization (CGH), microarrays, bead arrays, high-throughput genotyping technology, such as molecular inversion probe (MIP), and Genescan.

When using CGH arrays or when analyzing STRs or VNTRs, it is optional to compare a maternal blood sample enriched for fetal cells with one diluted such that it is statistically unlikely to have a fetal cell. Comparison of maternal and enriched samples allows for the detection of the presence of fetal cells and diagnosis of prenatal condition associated with the fetus.

Fetal abnormalities can also be detected using high throughput sequencing methods to identify SNPs and/or RNA expression patterns. Methods for quantifying nucleic acids are known in the art and include, but are not limited to, gas chromatography, supercritical fluid chromatography, liquid chromatography (including partition chromatography, adsorption chromatography, ion exchange chromatography, size-exclusion chromatography, thin-layer chromatography, and affinity chromatography), electrophoresis (including capillary electrophoresis, capillary zone electrophoresis, capillary isoelectric focusing, capillary electrochromatography, micellar electrokinetic capillary chromatography, isotachophoresis, transient isotachophoresis and capillary gel electrophoresis), comparative genomic hybridization (CGH), microarrays, bead arrays, and high-throughput genotyping such as with the use of molecular inversion probe (MIP).

Other analysis techniques include but are not limited to in situ hybridization techniques, RNA in situ fluorescence hybridization (RNA-FISH), in situ reverse transcription PCR, nucleic acid based amplification such as ligase chain reaction (LCR), transcription mediated amplification (TMA), strand displacement amplification (SDA), rolling-circle amplification (RCA), multiple-displacement amplification (MDA) or helicase-dependent amplification (HDA).

### RNA-FISH Assay for ε, γ, and β Transcripts

RNA-FISH probes can detect ε, γ, and β nuclear transcripts from fetal NRBC's and as such can be used to identify the presence of fnRBC's and/or distinguish fnRBC's from adult nRBC's. Sequences for gamma, epsilon and beta globin are available in Genbank and can be utilized to select the sequence regions that provide maximum sequence variation, such as intron and exon junctions. RNA-FISH probes for these regions can be prepared by any method known in the art including but not limited to nick-translation of PCR amplified DNA fragments (e.g., Invitrogen DNA FISH: Tag Kit), or in vitro transcription using PCR amplified DNA fragments tagged with a T7 RNA polymerase promoter consensus sequence and RNA FISH Tag Kit. In addition, the specificity of these RNA-FISH probes can readily be tested on hemin-induced K562 cells. Thus each probe's specificity, dynamic range, and limit of detection using ε, γ, and β cDNA in a dot blot assay can be determined. The probes that give the best signal to background ratio, the best limit of detection, and highest dynamic range, are selected for further use. The use of RNA-FISH probes is particularly useful for the detection of normal fetal female NRBC's in a sample of maternal RBC's. Preferably, the differential expression between the fetal transcripts and maternal transcripts is by a factor of at least 10, 50, 100, 500, 1000, or 5000. Using these methods >50, 60, 70, 80, 90, 85 or 99% of fetal cells can be identified by RNA-FISH and with less than 10, 1, or 0.1% of false positive readout.

The following oligonucleotides are contemplated herein for use as RNA FISH probes:

| RNA FISH | |
|---|---|
| probe for | Sequence |
| γ globin | CGACCTGGACTTTTGCCAGGCACAGGGTCC |
| | [SEQ ID NO: 1] |
| | TCACTCCCAACCCCAGTATCTTCAAACAGC |
| | [SEQ ID NO: 2] |
| | GCATCTTTTTAACGACCATACTTGTCCTGC |
| | [SEQ ID NO: 3] |
| | ACAGAGCTGACTTTCAAAATCTACTCCAGC |
| | [SEQ ID NO: 4] |
| γ globin | Genbank accession number HUMBB |
| | 48910-50766, 52388-54776 |
| β globin | TTCCACACTGATGCAATCATTCGTCTGTTT |
| | [SEQ ID NO: 5] |
| | TGTGTACACATATTAAAACATTACACTTTA |
| | [SEQ ID NO: 6] |
| | ATTAGCAATATGAAACCTCTTACATCAGTT |
| | [SEQ ID NO: 7] |
| | AGTAATGTACTAGGCAGACTGTGTAAAGTT |
| | [SEQ ID NO: 8] |
| β globin | Genbank accession number HUMBB |
| | 58041-61921 |

In some embodiments, probes are selected for detecting mRNA but not genomic DNA. Such probes have the highest sequence difference between gamma and beta globin. Such probes include primers that span over intron(s) sequence so that only mRNA but not genomic DNA is detected.

Examples of oligonucleotide or PNA probes that can be used for RNA FISH of γ globin include: (1) CTG GAC TAG GAG CTT ATT GAT AAC CTC AGA CGT TC [SEQ ID NO: 9], and (2) GCA GAA TAA AGC CTA TCC TTG AAA GCT CTG AAT CAT [SEQ ID NO: 10]. Examples of oligonucleotide or PNA probes that can be used for RNA FISH of β globin include: (1) AGG GAA CAA AGG AAC CTT TAA TAG AAA TTG GAC AGC [SEQ ID NO: 11], and (2) TTT GAG GTT GCT AGT GAA CAC AGT TGT GT [SEQ ID NO: 12].

Examples of in vitro transcribed RNA probes that can be used for RNA FISH of γ globin include (1) CTG GAC TAG GAG CTT ATT GAT AAC CTC AGA CGT TC [SEQ ID NO: 13]; and (2) GCA GAA TAA AGC CTA TCC TTG AAA GCT CTG AAT CAT [SEQ ID NO: 14]. Examples of in vitro transcribed RNA probes that can be used for RNA FISH of (globin include (1) AGG GAA CAA AGG AAC CTT TAA TAG AAA TTG GAC AGC [SEQ ID NO: 15]; and (2) TTT GAG GTT GCT AGT GAA CAC AGT TGT GT [SEQ ID NO: 16].

In some embodiments, probes are selected for detecting nuclear transcript (e.g., nuclear RNA or nRNA). Preferably, such probes do not bind mRNA. Such probes can be selected to bind intron-exon junction(s) that have the highest difference in exon sequences.

Examples of oligonucleotide or PNA probes for γ globin include CCT GGT CAC CAG AGC CTA CCT TCC CAG GGT [SEQ ID NO: 17] and for β globin CTT GTA ACC TTG ATA CCA ACC TGC CCA G [SEQ ID NO: 18]. Examples of an in vitro transcribed RNA probe that can be used for RNA FISH of γ globin include CCT GGT CAC CAG AGC CTA CCT TCC CAG GGT [SEQ ID NO: 19], and for β globin CTT GTA ACC TTG ATA CCA ACC TGC CCA G [SEQ ID NO: 20].

In any of the embodiments herein, the RNA FISH probe may be coupled to a molecular beacon for its detection and/or imaging. Examples of molecular beacons (labeling reagents) include but are not limited to chromophores, fluorescent moieties, enzymes, antigens, heavy metal, magnetic probes, dyes, phosphorescent groups, radioactive materials, chemiluminescent moieties, scattering or fluorescent nanoparticles, Raman signal generating moieties, and electrochemical detection moieties.

In one embodiment, maternal blood (20 mL) is diluted with an equal volume of Dulbecco's PBS (without Ca2+, Mg2+) with 1% BSA/2 mM EDTA (CSM buffer) and pumped into a CSM along with a parallel flow of CSM buffer. Typical operating pressures can range from about 0.6 to 1.2 PSI, yielding whole blood process rates of 5 to 15 mL/hr. The process can separate 99.5% of the nucleated cells from the diluted blood stream with minimal erythrocyte (99.995% RBC removal) or platelet contamination (>99.5% platelet removal) (Table 1).

Cells recovered from a CSM are treated with sodium nitrite to oxidize the hemoglobin, and applied to a MHEM (e.g., Miltenyi LS column outfitted with a specially configured magnetic circuit; FIG. 5). A CSM can comprise a flow restrictor to ensure that flow rates are sufficiently slow to promote cell capture (FIG. 5). Contaminating, unbound or loosely bound cells (primarily leukocytes) are removed by washing. Cells captured are released by removing the column from the system and eluting in the absence of the magnet field and are assessed for purity and cell structure by conventional histological stains (FIG. 5). Table 2 summarizes data obtained from the CSM run at 1 mL/hr. The CSM provided very high enrichment: removal of 100 billion RBCs, and collection of practically all the nucleated cells in a protein-free buffer of choice.

TABLE 2
Efficiency of the Cell Separation Module.

| | Distribution of cells | | |
|---|---|---|---|
| Fraction | Mean %: | SEM: | n: |
| % Nucleated cell retention (product) | 99.90% | 1.8 | 174 |
| % RBC removal (waste) | 99.99% | 0.03 | 167 |
| % Platelet removal (waste) | 99.90% | 0.7 | 138 |
| % Viability | 98.90% | 1.0 | 12 |

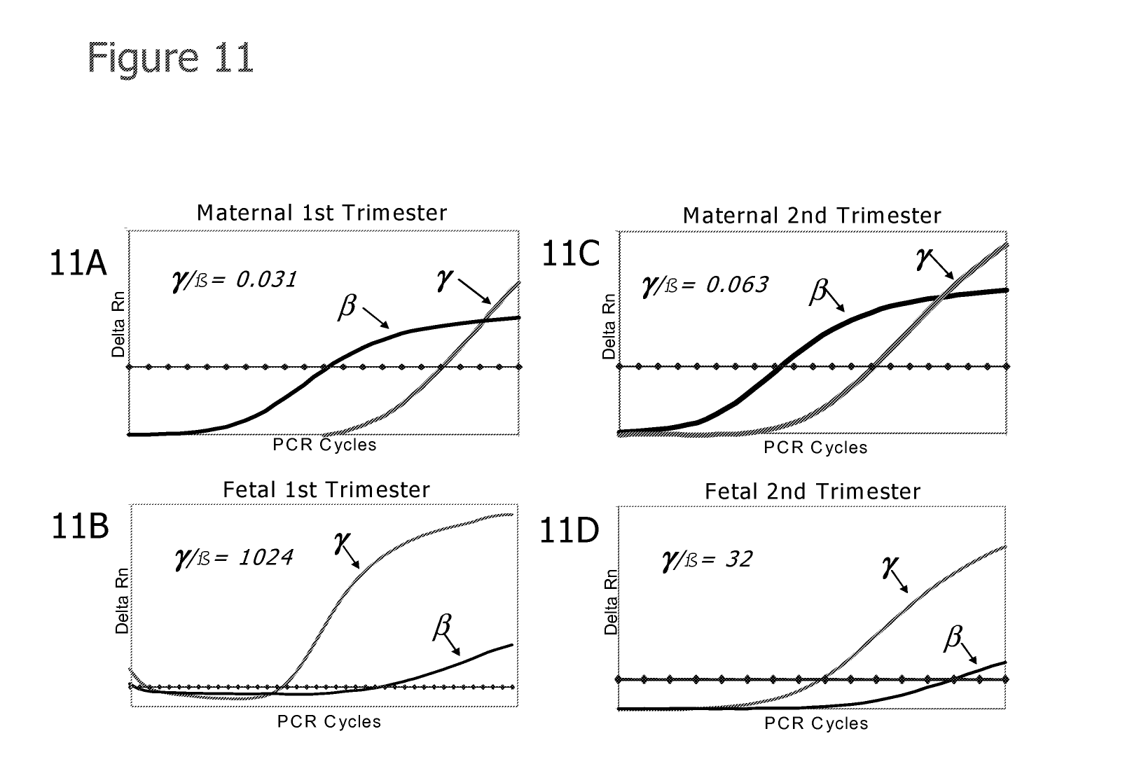

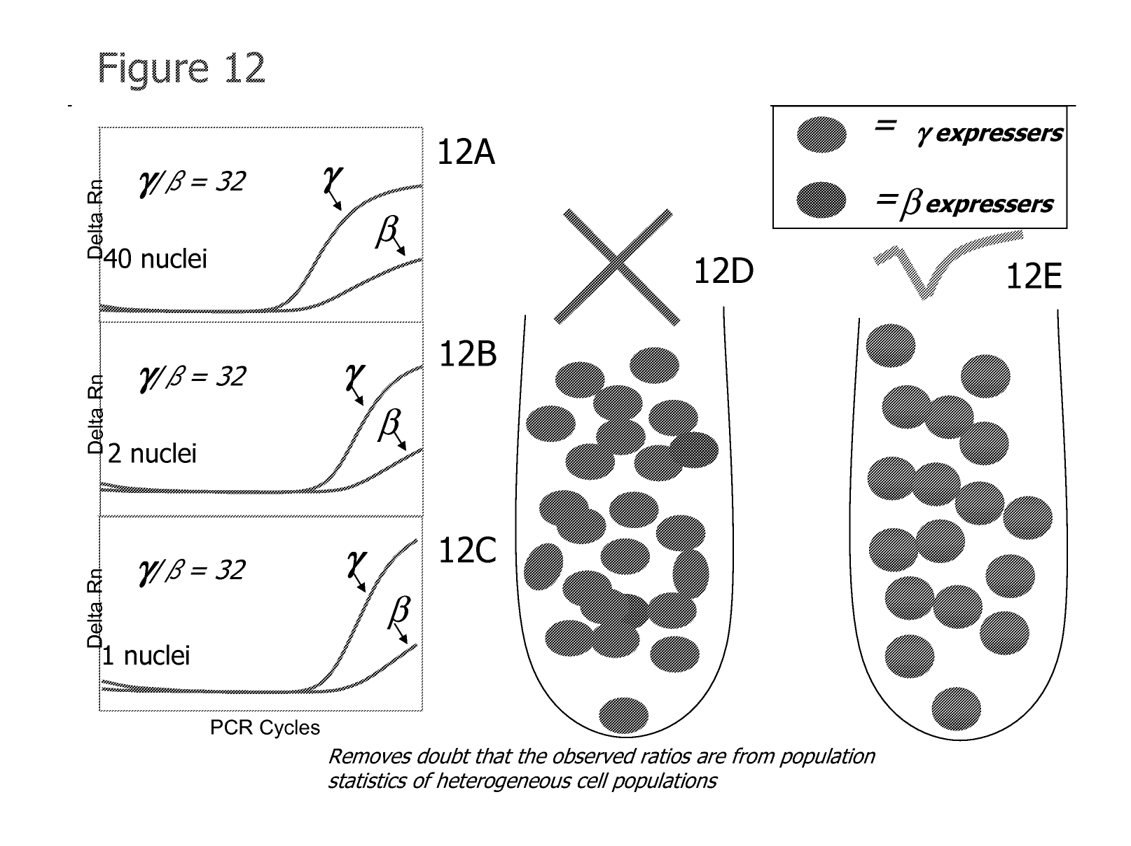

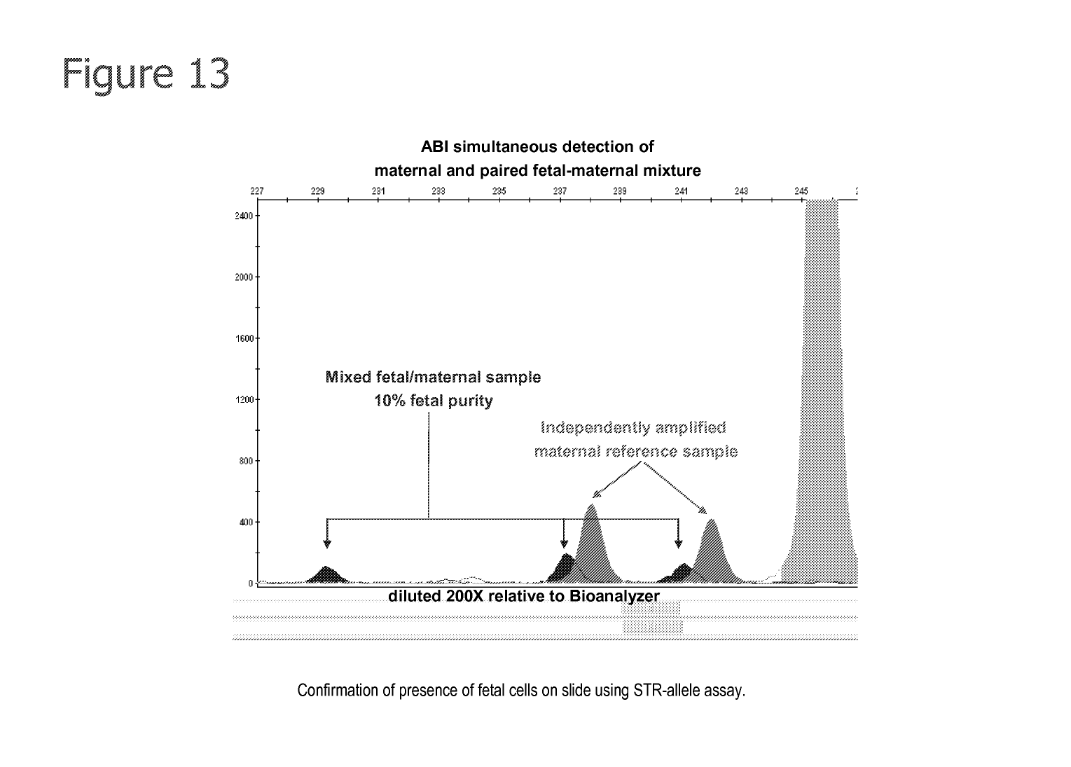

Cells are then lysed to release free nuclei which are cytospun onto glass slides. Subsequently, RT-PCR or RNA FISH as described herein can be utilized to distinguish fetal nuclei from adult nuclei based on measurement of differential expression for gamma/beta hemoglobins (FIGS. 11 and 12). Next the nuclei can be subject to DNA FISH to identify aneuploidy or Y probe to determine sex, for example. In addition, to confirm the fetal cell identification via biomarker ratio detection/measurement, STR allele analysis can confirm the origin of the cells (e.g., fetal versus adult).

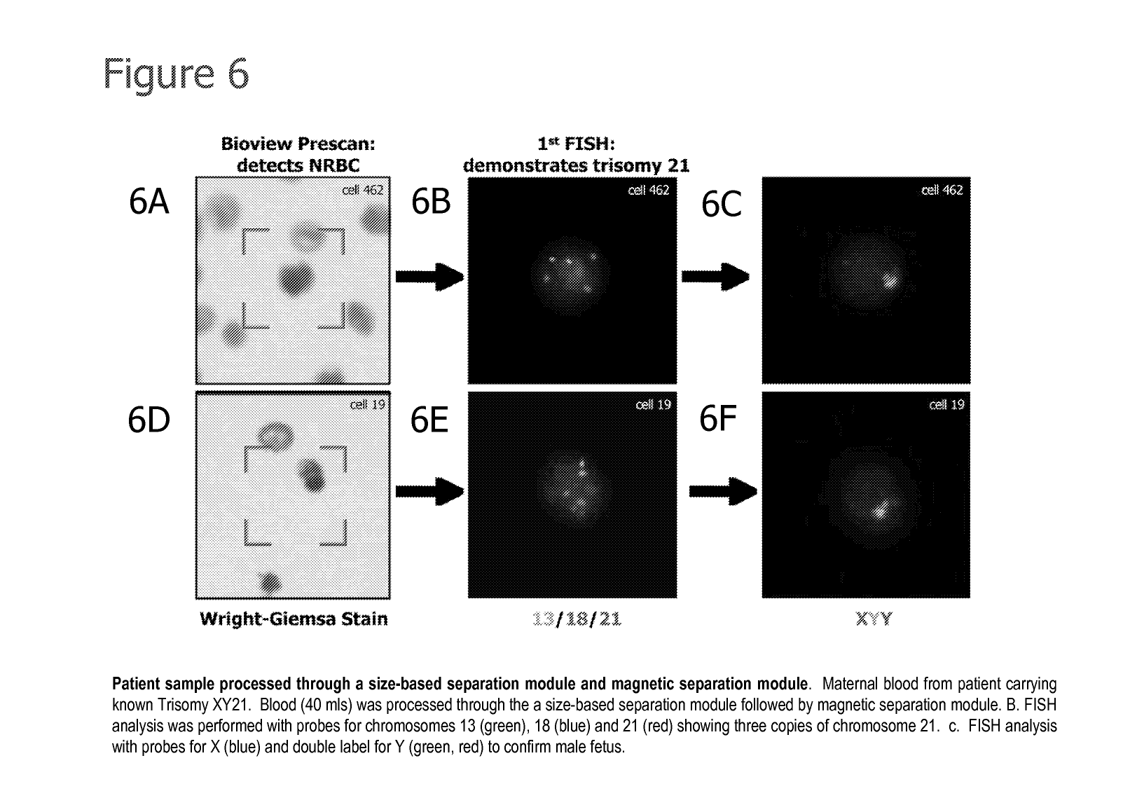

One illustration of fnRBC enrichment is powerfully demonstrated in FIG. 6, which contains a complete characterization of male fetal cells isolated from maternal blood which can be collected in the first or second trimester of pregnancy. First, the morphology of the Wright-Giemsa stained target cells was verified to be consistent with that of a NRBC, confirming that the target cells have been isolated. Next, cells are subjected to FISH with probes specific for the chromosomes of interest to look for particular genetic abnormalities—e.g., trisomy 21. Furthermore, the fetal origin of the abnormal cells was confirmed by successful hybridization of two Y chromosome specific FISH probes.

In one embodiment, a maternal blood sample (20 mL) populations of nRBCs that were enriched with the CSM and selected with the MHEM were treated in conventional ways to cytospin the cells onto poly-lysine coated glass slides and then subjected to fluorescence in situ hybridization (FISH) with oligonucleotide probes for chromosomes 21, X, and Y (FIG. 1, step 3). An automated fluorescence microscope (Bioview Inc., Israel) was used to scan the slides and identify aneuploid cells. As shown in Table 3, this process recovered substantial fnRBCs from each sample. The cells were of high quality enabling detection of Trisomy 21 in 5 out of 6 fetuses that had been diagnosed with Trisomy 21 by karyotype analysis (Table 3 and FIG. 6). Notably, no instances of false positives (calling a euploid 21 fetus a triploid for 21) were observed. The combination of more cells, and higher sensitivity and specificity, demonstrates the feasibility of using this novel microfluidic method for non-invasive prenatal testing.

TABLE 3
nRBC and fnRBC recovery with LMS Technology of CSM + Magnetic
Hemoglobin Enrichment + FISH Make confidential

Mean # cells/ml
(% aneuploid samples
detected)

nRBC Recovery

| | |
|---|---|
| LMS Process (n = 53) | 49 (100%) |

Post-termination fnRBC Recovery

| | |
|---|---|
| LMS Process (n = 15) | 18 (100%) |

Abnormal fetus fnRBC Recovery

| | |
|---|---|
| LMS Process (n = 6) | 0.8 (83%) |

### Labels and Probes

Labeled nucleic acid probes can be detected using any method known in the art including standard scanning equipment with either a scanning confocal laser or a charge coupled device (CCD) camera-based reader followed by spot identification using commercially or freely available software packages. Of course, a plurality of labels can be utilized and any conventional fluorochrome can be utilized in the invention. These are well known and commercially available. Specific examples of detectable molecules include radioactive isotopes such as p³² or H³, fluorophores such as fluorescein isothiocyanate (FITC), TRITC, rhodamine, tetramethylrhodamine, R-phycoerythrin, Cy-3, Cy-5, Cy-7. Texas Red, Phar-Red, allophycocyanin (APC), umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, Texas Red, dichlorotriazinylamine fluorescein, dansyl chloride, phycoerythrin, fluorescent lanthanide, as well as epitope tags such as the FLAG or HA epitope, and enzyme tags such as alkaline phosphatase, horseradish peroxidase, I²-galactosidase, and hapten conjugates such as digoxigenin or dinitrophenyl, etc. Other detectable markers include chemiluminescent and chromogenic molecules, optical or electron density markers, etc. The probes can also be labeled with semiconductor nanocrystals such as quantum dots (i.e., Qdots), described in U.S. Pat. No. 6,207,392. Qdots are commercially available from Quantum Dot Corporation. (See, Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland).

Furthermore, backbone labels that stain nucleic acid molecules in a sequence independent manner are also contemplated as probes herein. Examples of backbone labels include intercalating dyes such as phenanthridines and acidines (e.g., ethidium bromide, propidium iodide, hexidium iodide, dihydroethidium, ethidium homodimer-1 and -2, ethidium monoazide, and ACMA); some minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), and hydroxystilbamidine. The aforementioned nucleic acid stains are commercially available from suppliers such as Molecular Probes, Inc.

### Imaging Methodologies

Cells or nuclei of interest may be imaged using any suitable method. In one embodiment, cells or nuclei may be imaged by RNA-FISH with one or a combination of DNA probes, RNA probes, or synthetic oligo nucleotides. The cells or nuclei can be either in solution or on a slide. The probes can be prepared through nick translation of genomic or cDNA of selected marker genes, prepared through PCR amplification of the interested sequences, or RNA probes prepared through in vitro transcription, or synthetic oligo nucleotides with modified nucleotides, such as but not limited to locked nucleotide, o-methylated liked or peptide nucleotide acid, and synthetic oligo nucleotide without modified nucleotides. The probes, such as those that selectively bind RNA of beta, gamma, and epsilon globin genes can be selected to cover the region of sequence in such a way that only the spliced or matured mRNA can be detected or selected in such a way to detect the nascent unspliced RNA transcripts. Furthermore, the expression of multiple genes can serve as a marker of the presence (or absence) of fetal cells and probe(s) specific to such genes or transcripts thereof can be prepared as described above. These probes can be prepared with different labels, e.g. fluorescence labels, to facilitate identification. The cells or nuclei of interest can be identified based on the presence or absence of particular selected marker genes and the differential expression of selected marker genes. Additionally, probes can be selected from other target region, such as but not limited to genes on chromosome 13, 18, 21 and X, and these probes can be combined with the first set of probes, e.g., that detect the expression of, beta, gamma, and/or epsilon globins, to simultaneously identify the presence of fetal nucleated red blood cells and possible trisomy. Furthermore, the identified cells or nuclei can be isolated and subjected to genetic analysis to extract genetic information such as but not limited to chromosomal aneuploidy.

In one embodiment, the cells or nuclei of interest are identified through in situ reverse transcription polymerase chain reaction with selected marker genes, such as but not limited to beta, gamma, epsilon globin and fetal markers. The PCR primers are labeled with different fluorescence dyes to facilitate the PCR amplicon identification. The in situ rt-PCR can be performed in either solution or on slide. The target cells or nuclei of interest can be identified through FACS analysis of rt PCR reaction products to collect the target of interest or through imaging of sides, or cytospun RT-PCR reaction products on to a slides and then imaging the cells or nuclei. The presence and/or absence of particular marker gene or the differential expression of particular group of genes can be used to identify the target cells or nuclei of interest. The identified cells or nuclei can be further subjected to DNA FISH or isolated for extracting genetic information.

In one embodiment, the cells or nuclei of interest are identified through isothermal nucleic acid amplification of a group of selected marker genes, such as but not limited to beta, gamma, and epsilon globin. The isothermal nucleic acid amplification methodologies, such as but not limited to LCR, TMA, SDA, RCA, MDA, and HDA, can be employed are such that the selected marker genes are amplified proportionally to their individual dosage within the cells or nuclei. The amplification products can be directly detected through detecting the amplified labeled fluorescence dyes or hybridizing with fluorescence dye labeled probe specific to the amplified products and then measure the fluorescence dye labeled probes. The identified cells or nuclei can be further processed to extract genetic information.

The detection and analysis steps involve quantifying genomic DNA regions from cells in a sample or enriched sample. In some embodiments, the quantified genomic DNA regions are polymorphic sites such as short tandem repeats (STRs) or variable number of tandem repeats (VNTRs).

Polymorphic genomic DNA region(s) or whole genome(s) from the mixed sample (e.g., maternal blood sample enriched for fetal cells) and optionally reference (diluted maternal blood sample or sample having no fetal cells) are pre-amplified to increase the overall abundance of DNA used for quantification and analysis. Pre-amplification can be preformed using multiple displacement amplification (MDA) (Gonzalez et al. Envircon Microbiol; 7(7); 1024-8 (2005)) or amplification with outer primers in a nested PCR approach. This permits detection and analysis of fetal DNA even if the total amount of fetal DNA in the mixed (e.g. enriched) sample is only up to 1 pg, 500 ng, 200 ng, 100 ng, 50 ng, 40 ng, 30 ng, 20 ng, 10 ng, 5 ng, 1 ng, 500 pg, 200 pg, 100 pg, 50 pg, 40 pg, 30 pg, 20 p, 10 pg, 5 pg, or 1 pg or between 1-5 μg, 5-10 μg, or 10-50 μg. Pre-amplification allows the products to be split into multiple reactions at the next step.

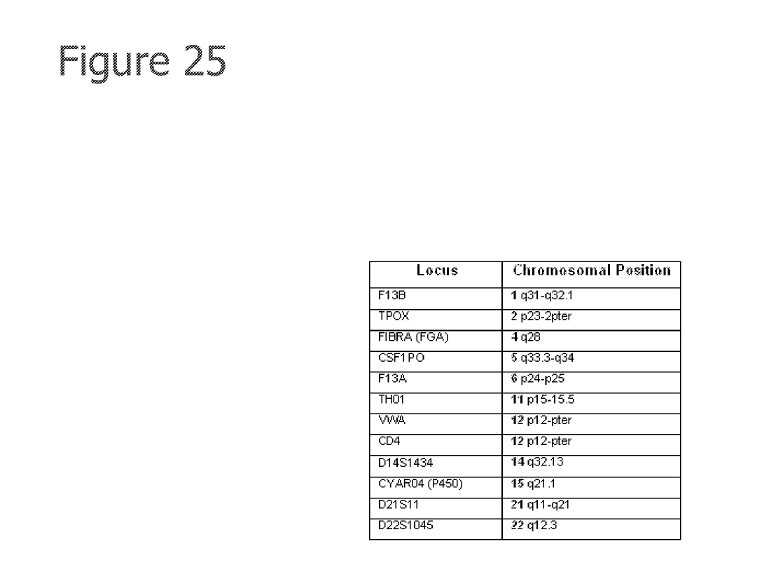

Polymorphic DNA region(s) such as short tandem repeats (STRs) or variable number of tandem repeats (VNTRs) are selected on suspected trisomic chromosome(s) (e.g., 13, 18, 21, X or Y) or chromosome(s) associated with a condition to be detected and optionally on control (non-trisomic) chromosomes. In some embodiments, 1 or more than 1, 2, 3, 4, 5, 6, 7, 8, 9, 10, 15, 20 DNA polymorphic loci are selected per target chromosome. Multiple polymorphic regions can be analyzed independently or at the same time in the same reaction. The polymorphic DNA regions, e.g. STRs loci, are selected for high heterozygosity (variety of alleles) so that the paternal allele of the fetal cells is more likely to be distinct in length from the maternal alleles. This results in an improved power to detect the presence of fetal cells in the mixed sample and potential fetal abnormalities in such cells. When the polymorphic regions selected are STR loci, di-, tri-, tetra- or penta-nucleotide repeat loci can be used for detection and analysis of fetal cells. Examples of STR loci include but are not limited to D21S1414, D21S1411, D21S1412, D21S11 MBP, D13S634, D13S631, D18S8535, AmgXY, XHPRT or a combination thereof, as well as those listed in FIGS. 25-27. Therefore, by detection of sex-linked STRs, in some embodiments, the methods of the invention allow for the determination of maternal or paternal trisomy.

In some embodiments, polymorphic loci selected are amplified. This amplification can be used to detect non-maternal fetal alleles in the mixed sample and to determine the copy number of such alleles. When amplifying more than one polymorphic locus or DNA region, primers are selected to be multiplexable (fairly uniform melting temperature, absence of cross-priming on the human genome, and absence of primer-primer interaction based on sequence analysis) with other primer pairs. Primers and loci are chosen so that the amplicon lengths from a given locus do not overlap with those from another locus.

In some embodiments, multiple dyes and multi-color fluorescence readout may be used to increase the multiplexing capacity, e.g. of a single CGE. This ensures that the loci are kept distinct in the readout (e.g. CGE readout). In such a case, PCR primer pairs can be grouped and the same end-labeling is applied to the members of a group.

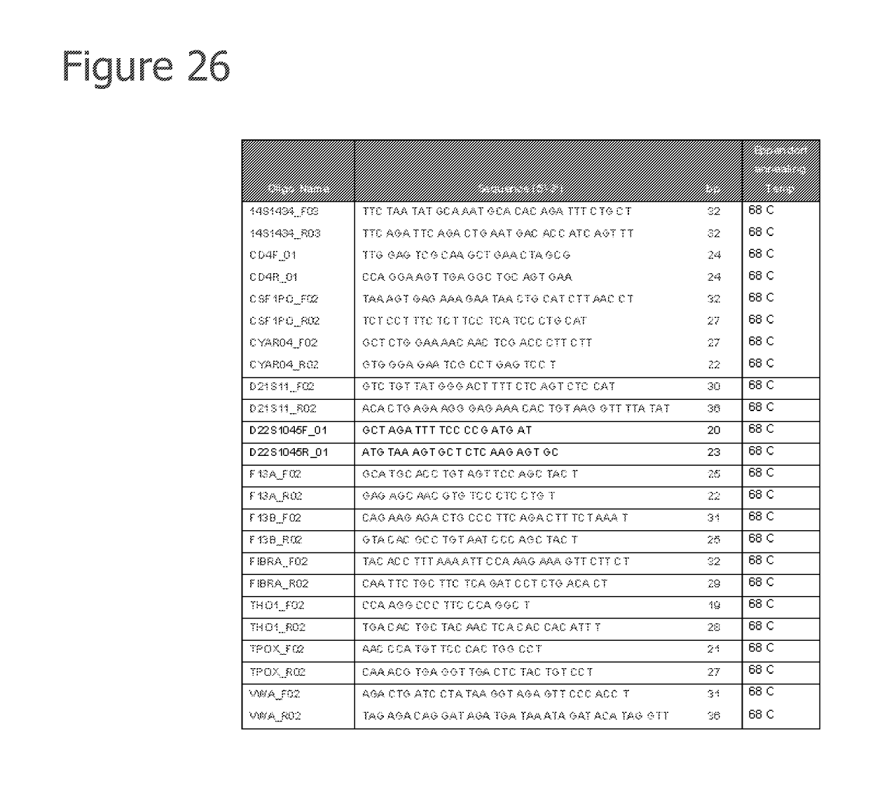

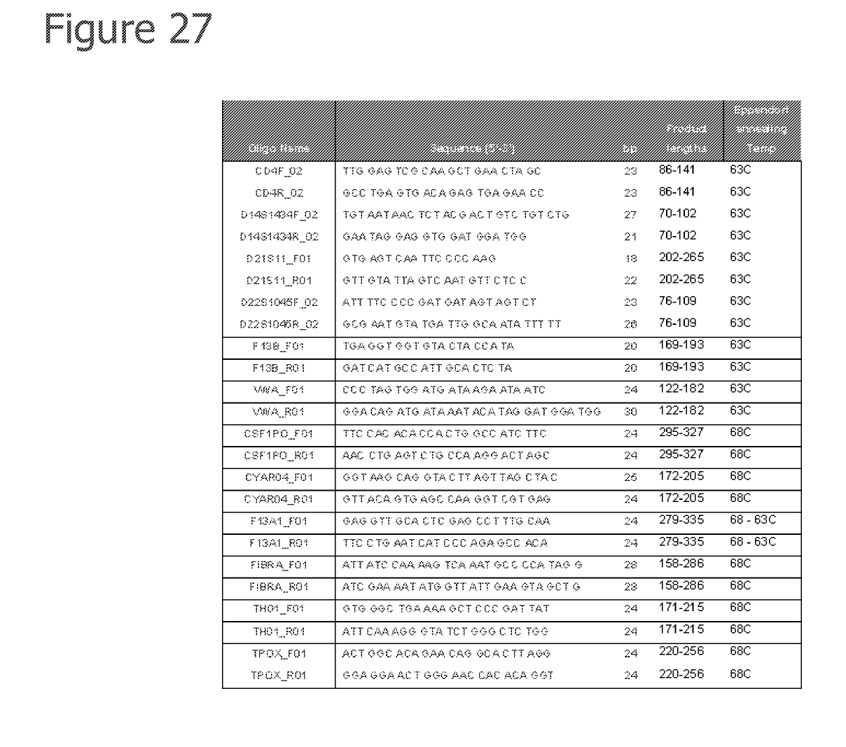

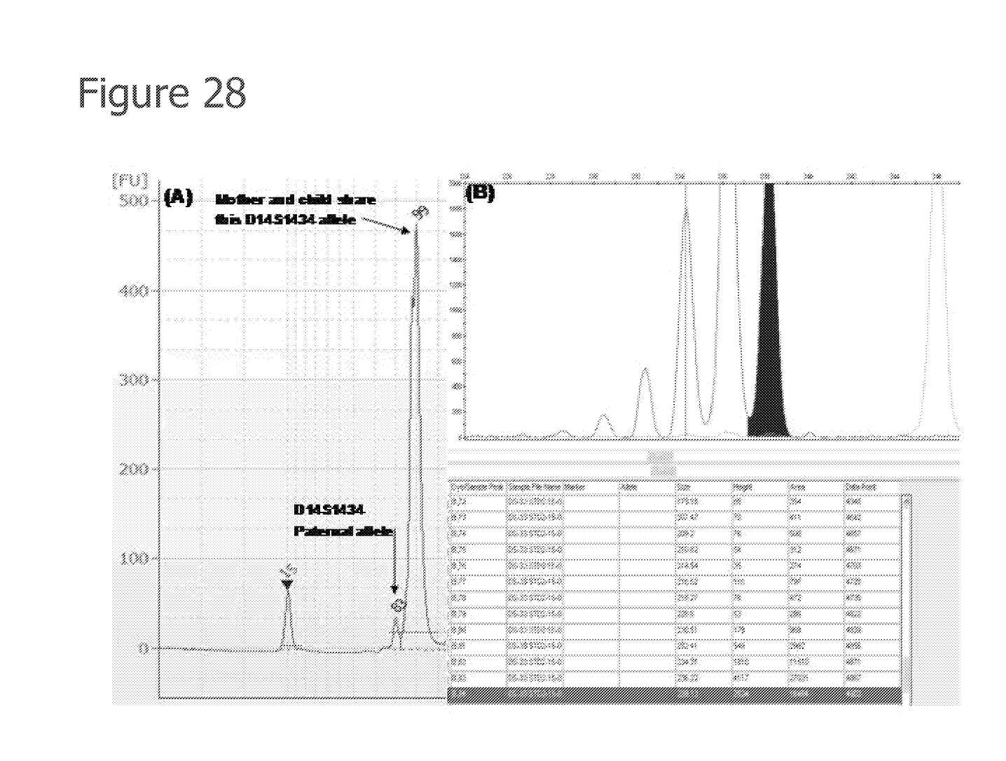

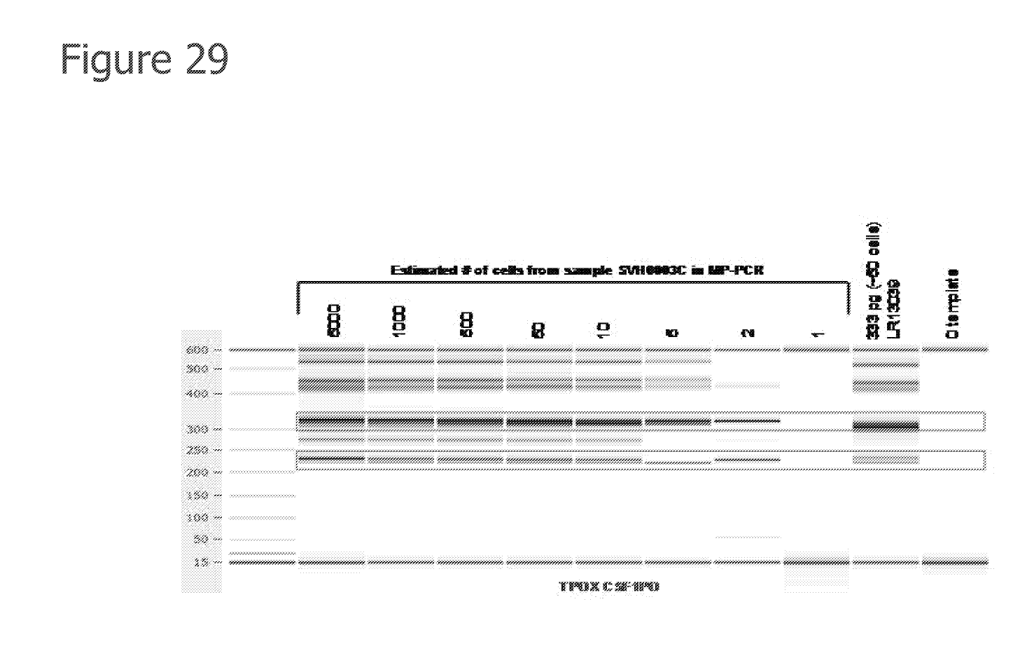

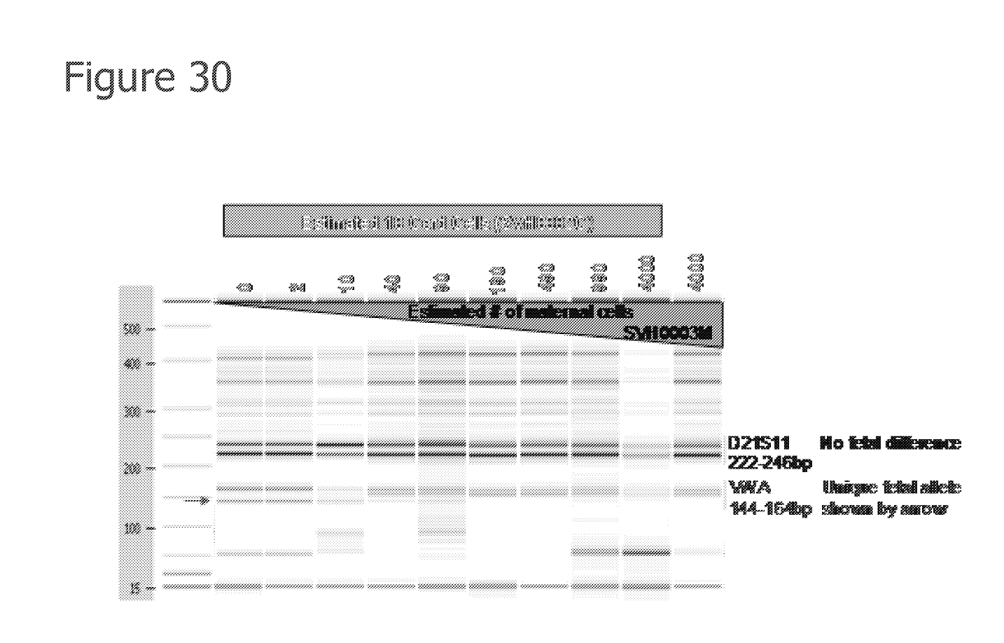

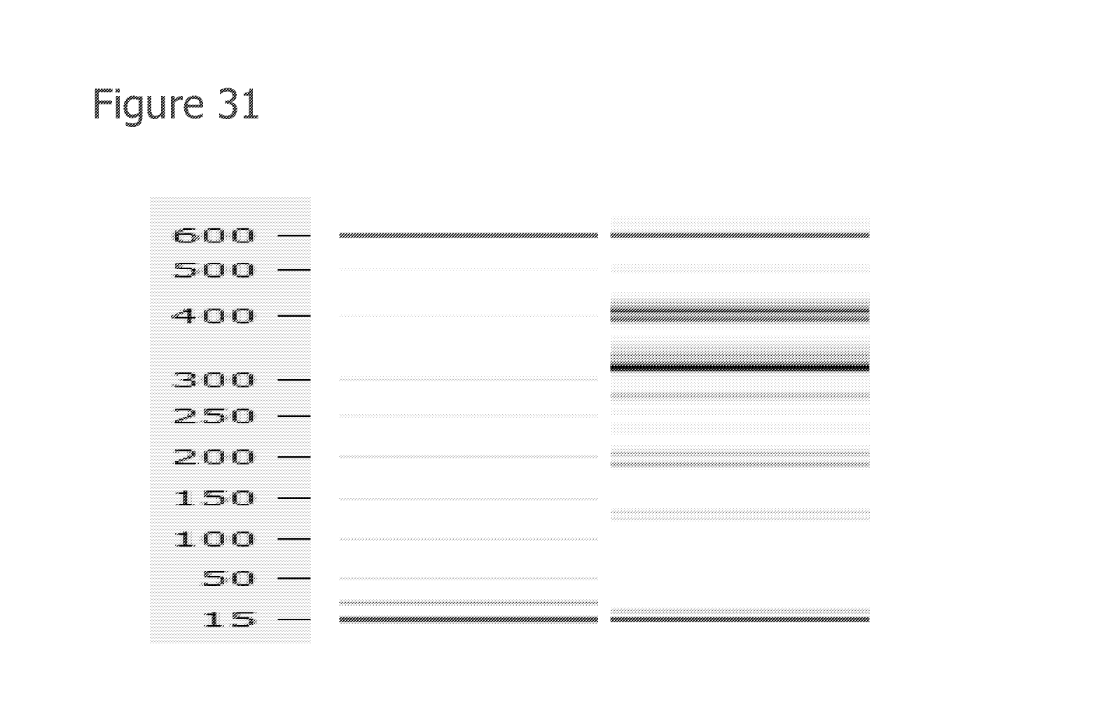

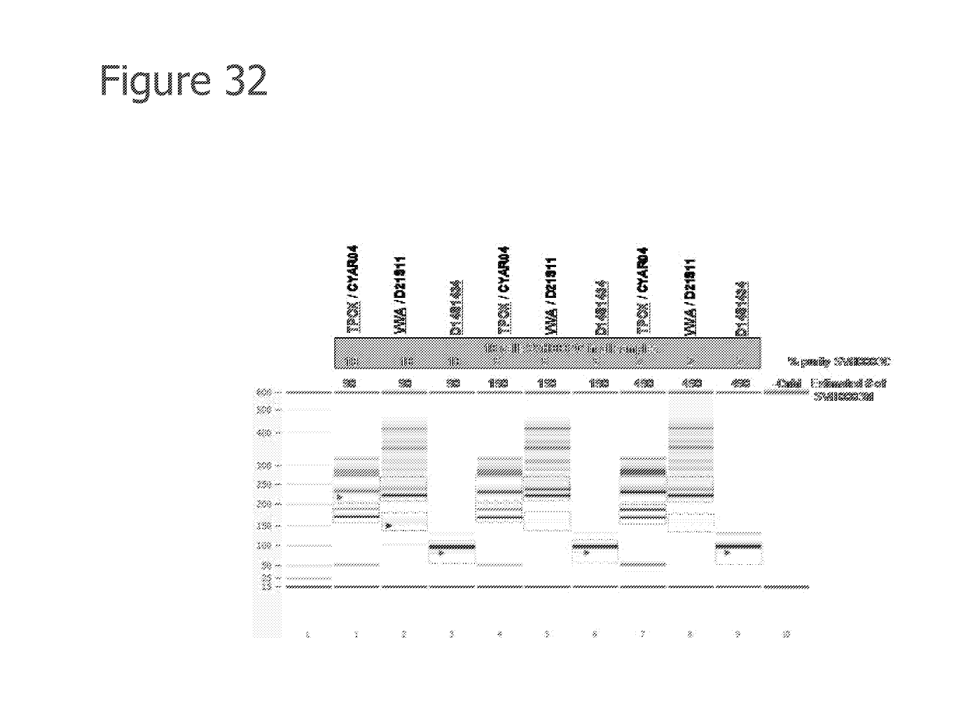

Examples of primers known in the art that correspond to specific STR loci that can be used in the present invention are described in FIGS. 26-27.

Examples of PCR techniques that can be used to amplify the DNA/RNA regions in methods of the invention herein include, but are not limited, to quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), bridge PCR, picotiter PCR and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that may be used to amplify specific polymorphic loci include those described in, U.S. Pat. Nos. 5,242,794, 5,494,810, 4,988,617 and 6,582,938.

The amplified DNA polymorphic regions (e.g. STR loci) from both mixed and reference samples are characterized and quantified using any method known in the art. Examples of such methods include, but are not limited to, gas chromatography, supercritical fluid chromatography, liquid chromatography, including partition chromatography, adsorption chromatography, ion exchange chromatography, size-exclusion chromatography, thin-layer chromatography, and affinity chromatography, electrophoresis, including capillary electrophoresis, capillary zone electrophoresis, capillary isoelectric focusing, capillary electrochromatography, micellar electrokinetic capillary chromatography, isotachophoresis, transient isotachophoresis and capillary gel electrophoresis, comparative genomic hybridization (CGH), microarrays, bead arrays, high-throughput genotyping technology, such as molecular inversion probe (MIP), and Genescan.

Nucleic acid probes can be utilized to determine RNA levels in a cell or nucleus. Examples of labels include, but are not limited to, chromophores, fluorescent moieties, enzymes, antigens, heavy metal, magnetic probes, dyes, phosphorescent groups, radioactive materials, chemiluminescent moieties, scattering or fluorescent nanoparticles, Raman signal generating moieties, and electrochemical detection moieties. For example, nucleic acid probes can be labeled with either Cy3 or Cy5-dUTP using random priming and are subsequently hybridized onto a sample in a solution containing an excess of Cot1-DNA to block repetitive sequences. Hybridizations can either be performed manually under a coverslip, in a gasket with gentle rocking or, automatically using commercially available hybridization stations. These automated hybridization stations allow for an active hybridization process, thereby improving the reproducibility as well as reducing the actual hybridization time, which increases throughput.

### Genetic Analysis

In various embodiments, isolated target cells are subjected to one or more genetic analyses. For example, in one embodiment, capillary gel electrophoresis (CGE) is used to quantify STRs in both the mixed and reference samples. This can be used to detect non-maternal fetal alleles in the mixed sample and to determine the copy number of such alleles. The mixed sample and the reference sample can be analyzed in separate reactions, e.g. separate CGE lanes. Alternatively, the mixed and the reference sample can be run in the same reaction, e.g. same CGE lane, by using two different dye labels, e.g. differently labeled PCR primers. When a reference sample is run through the PCR/CGE process, the alleles show up as peaks in the CGE. It is desirable, but not essential, to associate these peaks with known alleles in the population at each locus. When performing PCR/CGE it may be useful to reduce non-linearities in the response of PCR to input DNA copies (i.e. to effect more quantitative PCR) so that the data can be more easily related to models of aneuploidy. This ‘linearization’ can be accomplished by the following procedure: (a) the PCR reaction is initiated; (b) the PCR reaction is interrupted after N cycles (N=5 to 10) and ˜one third of the reaction products are removed and run on CGE. PCR cycling is re-initiated. Repeat until 40 PCR cycles or saturation is achieved; (a) CGE peak masses are determined and normalized to correct for the depletion of the reaction products at each iteration of (b) a saturation (splining) curve is fit to the normalized data for each allele peak, and quantitative starting concentrations are inferred as in customary qPCR.

The above procedure tends to accomplish quantitative PCR while enabling a high degree of multiplexing. Because each CGE run has a slightly different relation between DNA fragment size (and sequence) and mobility, each trace typically needs to undergo a length transformation, such as a low-order (cubic or quartic) polynomial transformation, in order to map to the data from the trace corresponding to the previous amplification point. This mapping can be determined by adjusting the transformation parameters to achieve the best fit of the one data trace to the other, with both normalized to the same total sum of squares or summed peak heights.

The maternal peaks at each locus provide an estimate of the secondary ‘stutter’ structure at each locus due to PCR errors. The locations of these small secondary peaks can be used to blank out length regions that are contaminated by this stutter when looking for and using the non-maternal allele peaks (as described herein for example). Alternatively, more sophisticated ‘deconvolution’ algorithms can be applied to remove the stutter (e.g., Stoughton, et al., Electrophoresis; 18(1): 1-S (1997)).

The sample containing an unknown mixture of fetal and maternal cells is analyzed as in Step (2). This could be done in a separate CGE lane, or in the same CGE lane as the maternal sample by using two different dye labels on the PCR primers. Because each CGE trace has a slightly different relation between DNA fragment size (and sequence) and mobility, these data typically needs to undergo a length transformation, such as a low-order (cubic or quartic) polynomial transformation in order to map one trace onto the other to facilitate peak identification and model fitting. This mapping can be determined by adjusting the transformation parameters to achieve the best fit of the peak locations in one data trace to the other. This mapping can be well determined in the assumed situation where the maternal cells are more numerous than the fetal cells, because the maternal signature can dominate and can be shared in the two data sets.

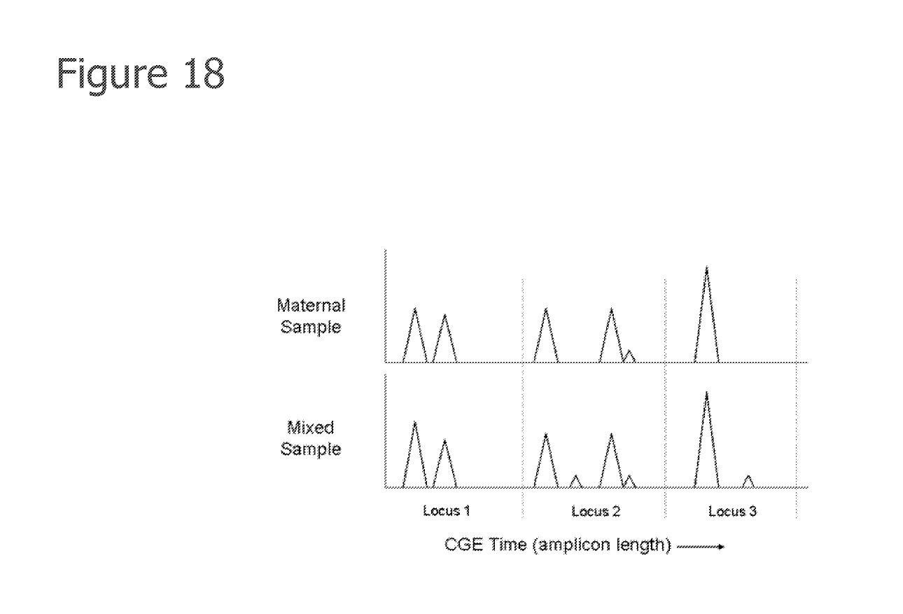

FIG. 18 illustrates typical locus patterns arising from a normal (diploid) fetus and mother. At Locus 1, the paternal allele is the same as the left hand maternal allele, and adds to its apparent height. At Locus 2, the paternal allele has a length between the lengths of the maternal alleles. In addition, there is a secondary ‘stutter’ peak on the shoulder of the right hand maternal peak. In Locus 3, the maternal sample is homozygous leading to only one main peak, and the paternal allele is distinct from this allele.

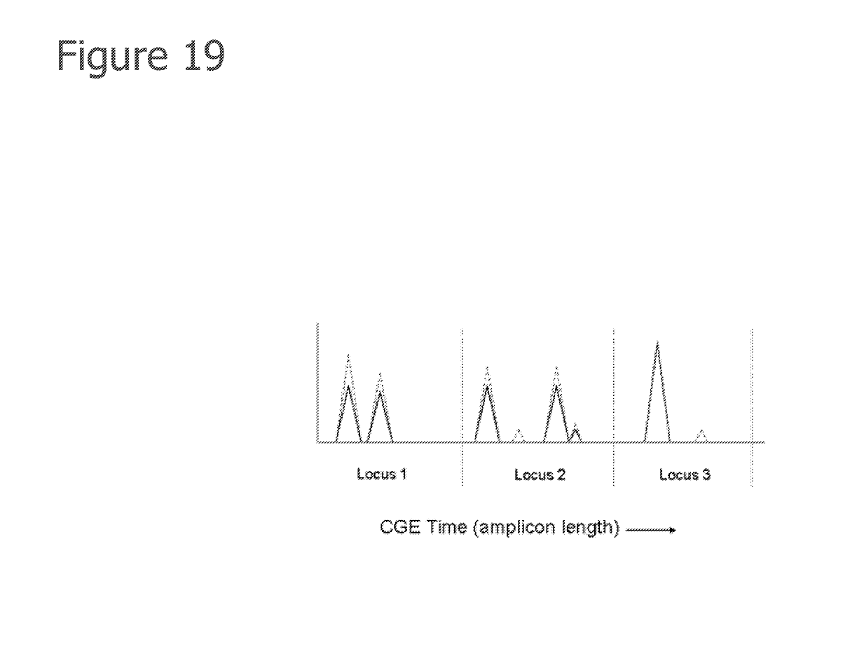

FIG. 19 illustrates locus patterns arising from trisomic fetal cells. The dashed trace represents mixed sample containing trisomic fetal cells, superposed on maternal sample trace (solid black). Trisomy causes excess amplitude in maternal alleles at loci contained within the aneuploid region (here assumed to contain Loci 1 and 2 but not Locus 3). The left hand maternal peak at Locus 1 contains contributions from the trisomy and from a paternal allele.

From the data obtained from the quantifying step different data models can be constructed depending upon different assumptions. For example, a data model for the CGE patterns in FIGS. 18 and 19 can be as follows:

Let m1 denote the CGE signal obtained from one of the maternal alleles at a given locus and m2 the signal obtained from the other maternal allele, which might be the same allele. Let p denote the CGE signal obtained from the paternal allele at a given locus. Let p1 and p2 denote the CGE signals obtained from the paternal alleles at a given locus when a paternally derived trisomy occurs. Let α and β denote the relative number of maternal and fetal cells, respectively. Then in the case of a chromosome with maternal non-dysjunction trisomy, the data can have the form

x=α(m1+m2)+β(m1+m2+p). (1)

A normal (diploid) chromosome can give

x=α(m1+m2)+β([m1 or m2]+p), (2)

and a paternally derived trisomy can give

x=α(m1+m2)+β([m1 or m2]+p1+p2). (3)

In some embodiments, data and data model is represented as discrete peak masses (or heights) and peak locations or as vectors of values representing the actual peak profiles. In the case of representation by peak characteristics, the ‘addition’ operation in Equations 1-3 denotes summation of peak height or mass at the discrete allele location. In the case where the full peak profiles are represented, summation denotes summation of signals bin by bin over the CGE trace, and in this case it may be helpful to zero the data except in the immediate vicinity of actual peaks. Representation via peak characteristics is preferable when using the PCR linearization technique described above.

To determine aneuploidy, the difference between the structure of the β-term that appears in the first and second equations above is determined. In the first case, there is an additional contribution to both maternal alleles along with the paternal allele, and in the second case there is an additional contribution only to one of the maternal alleles along with the paternal allele. The essence of the presence/absence declaration for fetal cells lies in the evidence for P being greater than zero.

The best overall fit of model to data is selected from among all the model sets. This modeling approach optimally uses information contained in the increase of chromosome copy number with aneuploidy and its association with the strength of non-maternal alleles.

In some embodiments CGE signals representing m1 and m2 at each locus are obtained by profiling the maternal-only sample and mapping the peak locations to the corresponding ones of the mixed sample. The heights of m1 and m2 may be unequal, and this helps correct for PCR amplification biases associated with particular alleles. The values of p, p1, p2, α, and β are determined from the mixed sample data by fitting Equations 1-3 to the data, optionally by using the least squares, or the maximum likelihood methods.

The three models need to be fit to each chromosome with suspected trisomy, e.g. chromosomes 13, 18, 21, x and/or y. If there are only 3 suspected chromosomes, this results in 27 model variants (3×3×3=27). In Equations 2 and 3, there is also the ambiguity between using m1 or m2 in the β-term, so there are 5 model variants for each chromosome, with 5×5×5=125 total variants over three suspected trisomy chromosomes.

Segmental aneuploidies also could be tested by hypothesizing that different contiguous subsets of loci are contained within the aneuploid region. With each model variant, α and β have to be determined and the parameters describing the paternal alleles have to be determined at each locus for each model variant. The paternal allele peak height and shape can be assumed to be an average of the known maternal ones at that locus, while the paternal allele location needs to be fit to the data. The possible locations for the paternal allele can be the location of m1, the location of m2, and ‘elsewhere in the locus window’ where this latter possibility involves a search over discrete shifts smaller than a typical peak half-width at half maximum. Prior probabilities on the choices of p, taken from population allele frequency data, can be used, if their product lengths can be predicted.

In some cases, because of the number of parameters being fit, suboptimal searches can be used for computational efficiency. For example, one possible approach involves iterative methods, such as the following, which would be applied to each data model variant: (i) Set β to 0 and solve for a; (ii) Set β/α to a value where β/α is the smallest fetal/maternal cell ratio for which fetal cells are likely to be detectable; (iii) Solve for paternal allele location(s) at each locus, one locus at a time that minimize data-model residuals; (iv) Fix the paternal, allele parameters and adjust β to minimize residuals over all the data; (v) Now vary only a to minimize residuals; (vi) Repeat iv and v until convergence; and (vii) Repeat iii through v until convergence.

The presence or absence of fetal DNA is determined using the models described above. The best overall fit for such models yields the values of β, α that can be called βₘₐₓ, αₘₐₓ. The likelihood of observing the data given βₘₐₓ can be compared to the likelihood given β=0. The ratio is a measure of the amount of evidence for fetal DNA. A threshold for declaring fetal DNA is the likelihood ratio of approximately 1000 or more. The likelihood calculation can be approximated by a Chi-squared calculation involving the sum of squared residuals between the data and the model, where each residual is normalized by the expected rms error.

If it is determined that fetal DNA is not present in the mixed sample as calculated above, then the test is declared to be non-informative. On the other hand, if it is decided that fetal DNA is present in the mixed sample, then the likelihoods of the data given the different data model types can be compared to declare trisomy or another condition.

The likelihood ratios of trisomy models (Equations 1 and 3) to the normal model (Equation 2) are calculated and these ratios are compared to a predefined threshold. This threshold can be set so that in controlled tests all the trisomic cases are declared aneuploid, and so that it is expected that the vast majority (>99.9%) of all truly trisomic cases are declared aneuploid by the test. In one embodiment, to accomplish a detection rate of >90% or 95% or approximately 99.9%, the likelihood ratio threshold is increased beyond what is necessary to declare all the known trisomic cases in the validation set by a factor of 1000/N, where N is the number of trisomy cases in the validation set.

Errors that may arise from the experimental procedure used to obtain the data can be taken into account in the model calculation(s). For instance, in the example described above, CGE data contain small additive errors associated with CGE readout, and larger multiplicative errors associated with PCR amplification efficiencies being different from locus to locus and from allele to allele within a locus. By using the maternal-only data to define m1 and m2 peak characteristics at each locus, the effects of PCR amplification biases associated with different primers and different amplicons from the same primers have been mostly controlled. Nevertheless, small variations in the process from day to day and the statistics of small numbers of starting genome copies can cause some random errors to remain. These tend to be multiplicative errors in the resulting CGE peak heights; e.g. two peaks may be 20% different in height although the starting concentrations of the alleles were identical. In one embodiment, it may be assumed that errors are random from peak to peak, and have relatively small additive errors, and larger Poisson and multiplicative error components. The magnitudes of these error components can be estimated from repeated PCR/CGE processing of identical samples. The Chi-square residuals calculation for any data-model fit then can be supported with these modeled squared errors for any peak height or data bin.

In another aspect of the present invention, the presence of fetal cells in a mixed sample and fetal abnormalities in said cells is determined without trying to integrate them in a data-model fitting procedure described above. Then, analysis using Equations 1 and 2 focuses on two indications. First, aneuploidy results in an excess of DNA for the trisomic chromosome, and this is indicated by the difference in mean strengths of the alleles on the trisomic chromosome compared to control chromosomes. A t-test can be applied to the two distributions of m1 and m2 peak heights. These peak heights are normalized to (e.g., divided peak-by-peak by) the corresponding peaks in the maternal-only sample to reduce PCR amplification biases. Second, Equations 1 and 2 show that aneuploidy is associated with less inequality in the heights of m1 and m2 at a given locus, particularly for loci where the paternal allele is distinct from the maternal alleles. Loci are selected where a third (paternal) allele is visibly distinct from two maternal alleles, and the distribution of the inequalities (measured in %) between the m1 and m2 peaks are compared between suspected trisomy chromosomes and control chromosomes. Again, peak heights first are normalized by the maternal-only sample. These two lines of evidence are combined to create an overall likelihood, such as by multiplying the probability values from the two lines of evidence. The presence/absence call is done in a simplified way by looking for loci where a third allele is clearly visible, and comparing the distribution of these peak heights between the maternal and mixed samples. Again, a t-test between these distributions gives the probability of fetal DNA being present.

In another aspect of the invention, the methods herein only determine presence or absence of fetal DNA, and aneuploidy information is known from another sources (e.g. fluorescence in situ hybridization (FISH) assay). For example, it may be desirable only to verify the presence or absence of fetal cells to ensure that a diploid test result is truly due to a normal fetus and not to failure of an assay (e.g. FISH). In this case, the process may be simplified by focusing on detecting the presence of non-maternal alleles without regard to associating them with increases in the maternal allele strengths at the same locus. Thus a process similar to the one outlined above may be used but it is not as necessary to arrange the PCR product lengths so that the products from different loci have distinct length windows in the CGE readout. The alleles from the different loci can be allowed to fall essentially anywhere in the effective measurement length window of the CGE. It also is not necessary to ‘lineate’ the PCR result(s) via multiple CGE readouts at different stages in the PCR cycling.

Therefore, maternal-only and mixed samples are run and mapped to each other to align maternal allele peak locations, as described above. PCR is run to saturation, or nearly to saturation, to be sure to detect the low abundance fetal sequences. The evidence for fetal DNA then arises from extra peaks in the mixed-sample data with respect to the maternal-sample data. Based on typical heterozygosities of approximately 0.7 for highly polymorphic STRs, the chance of not seeing a distinct paternal allele (distinct from both maternal alleles) when fetal DNA is in fact present decreases approximately as (0.7²)^{N}, where N is the number of loci included. Thus approximately ten STR loci can provide 99.9% probability of detection. In addition, the present invention provides methods to determine when there are insufficient fetal cells for a determination and report a non-informative case. The present invention involves quantifying regions of genomic DNA from a mixed sample. More particularly the invention involves quantifying DNA polymorphisms from the mixed sample. In some embodiments, one or more than 1, 2, 3, 4, 5, 6, 7, 8, 9, 10, 15, 20 DNA polymorphism loci (particularly STRs) per target chromosome are analyzed to verify presence of fetal cells.

Any of the steps above can be performed by a computer program product that comprises a computer executable logic that is recorded on a computer readable medium. For example, the computer program can execute some or all of the following functions: (i) controlling enrichment of fetal cells or DNA from mixed sample and reference sample, (ii) pre-amplifying DNA from both samples, (iii) amplifying specific polymorphic DNA regions from both samples, (iv) identifying and quantifying maternal alleles in the reference sample, (v) identifying maternal and non-maternal alleles in the mixed sample, (vi) fitting data on alleles detected from mixed and/or reference samples into data models, (vii) determining the presence or absence of fetal cells in the mixed sample, (viii) declaring normal or abnormal phenotype for a fetus based on data models or declaring non-informative results, and (ix) declaring a specific fetal abnormality based on the above results. In particular, the computer executable logic can fit data on the quantity of DNA polymorphism(s) (e.g. STR's) into one or more data models. One example of a data model provides a determination of a fetal abnormality from given data signals obtained by molecular analysis e.g. CGE. The computer executable logic provides for a determination of the presence or absence of a trisomy, and distinguish whether the trisomy is paternally derived or if it originates from a maternal non-disjunction event.

For example, given the following data signals obtained through molecular analysis (e.g. CGE): m1, which represents a signal obtained from one of the maternal alleles (m1) at a given locus, m2, which represents a signal obtained from the other maternal allele, which might be the same allele, p, which is a signal that is obtained from the paternal allele at a given locus, and p1 and p2, which are signals obtained from the paternal alleles at one given locus when a paternally derived trisomy occurs, and letting α and β, which denote the relative number of maternal and fetal cells, respectively, the following determinations can be made. In the case of a chromosome with maternal non-disjunction trisomy, the data can have the form x=α(m1+m2)+β(m1+m2+p). (1) A normal (diploid) chromosome can give x=at m1+m2)+β([m1 or m2]+p). (2) and a paternally derived trisomy can give x=α(m1+m2)+β([m1 or m2]+p1+p2). (3) The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine.

The program can provide a method of evaluating the presence or absence of trisomy in a mixed cell sample by accessing data that reflects the level of polymorphism(s) at two alleles at two or more given loci in a mixed sample (maternal and fetal cells) and in a sample enriched in fetal cells, relating the levels of polymorphism(s) to the number of maternal and fetal cells (α and β in equations 1-3), and determining the presence or absence of trisomy in the samples.

In one embodiment, the computer executing the computer logic of the invention may also include a digital input device such as a scanner. The digital input device can provide information on the polymorphism levels/quantity. For example, a scanner of this invention can provide an image of the DNA polymorphism (particularly STRs) according to method herein. For instance, a scanner can provide an image by detecting fluorescent, radioactive, or other emission; by detecting transmitted, reflected, or scattered radiation; by detecting electromagnetic properties or other characteristics; or by other techniques. The data detected is typically stored in a memory device in the form of a data file. In one embodiment, a scanner may identify one or more labeled targets. For instance, a first DNA polymorphism may be labeled with a first dye that fluoresces at a particular characteristic frequency, or narrow band of frequencies, in response to an excitation source of a particular frequency. A second DNA polymorphism may be labeled with a second dye that fluoresces at a different characteristic frequency. The excitation sources for the second dye may, but need not, have a different excitation frequency than the source that excites the first dye, e.g., the excitation sources could be the same, or different, lasers.

Another aspect of the invention includes kits containing the devices and reagents for performing the enrichment and genetic analysis. Such kits may include the materials for any individual step disclosed, any combination of devices and reagents or the devices and reagents for performing all of the steps. For example, a kit may include the arrays for size-based enrichment, the device for magnetic separation of the cells and reagents for performing PCR or CGE. Also included may be the reagents for performing multiple displacement amplification. This is an exemplary kit and the kits can be constructed using any combination of disclosed materials and devices. The use of the size-based enrichment provides gentle handling that is particularly advantageous for permitting subsequent genetic analysis.

In one embodiment, a kit comprises nucleic acid probes/primers for detection of.

### EXAMPLES

### Example 1

### RNA FISH Analysis

After fetal cells are enriched by one or more of the methods described herein, RNA FISH analysis is performed using methods disclosed in Raamsdonk et al., 2001, NAR; 29:8:e42 or as described below:
1. Cytospin the cells onto a glass slide (or cell grow on slide).
2. Permeabilize the cells with cytoskeletal buffer (CB buffer 100 mM NaCl, 30 mM sucrose, 3 mM MgCl2, 10 mM PIPES, pH 6.8), 30 s (CB buffer can be replaced with 1×PBS).
3. CB+0.5% Tx-100, 30 s
4. CB buffer for 30 s.
5. Fix in 4% paraformaldehyde in 1×PBS for 10 min
6. 70% ethanol at 4 C for up to 2 weeks
7. or dehydrate the slides with 70, 80, 90, 100% ethanol, and dried
8. Hybridize with probes on at 37 C in 50% formamide, 6×SCC
9. Slides wash in 50% formamide,
10. 2×SSC at 39 C for 3×5 min,
11. 2×SSC at 37 C for 3×5 min,
12. Room temperature using 1×SSC 10 min
13. 4×SSC 5 min

Preparation of Probe:
1. Probes are prepared using Boehringer Mannheim nick translation kit or Stratagene random primer labeling kit, essentially as recommended by manufacturer, with either biotin-16-dUTP or digoxigenin-11-dUTP. After labeling, the probes are precipitated with mouse Cot1 DNA or yeast tRNA (as appropriate based on the presence or absence of cross-hybridizing repetitive DNA), 0.3M Na-acetate and 2 volumes EtOH at 4° C. for 1 h. The probes will be spun and resuspended in hybridization buffer at 2 ng/μl probe DNA. Hybridization buffer: 50% formamide (American Bioanalytical), 2×SSC-pH7.4 (autoclaved), 2 mg/ml BSA (Boehringer Mannheim), 10% Dextran sulfate-500K (autoclaved).
2. For hybridization, the probe is denatured 75° C., for 10 minutes.
3. If probe contains repetitive elements, the probe is preannealed at 42° C., 10-60 minutes using a Cot-1 cocktail (length of preannealling depends upon the repeats present).

Preparation of Slides:
1. Cells can be isolated either by lysis to remove enucleated RBC with methanol:acetic alcohol (3:1) or by directly cytospinning onto a glass slide.
2. Slides are fixed in cleaned Coplin jars as follows:
   a. Immerse slides for 5 min. in ice-cold PBS.
   b. Transfer to ice-cold CSK buffer (100 mM NaCl, 300 mM sucrose, 10 mM PIPES-pH6.8, 3 mM MgCl₂) for 60 sec.
   c. Immerse in CSK+0.5% Triton for 60 sec.
   d. Re-equilibrate in CSK for 60 sec.
   e. Fix in 4% paraformaldehyde in 1×PBS, pH 7.4 for 10 min. at room temperature. (dissolve in 4 mM NaOH at 50° C., add 1/10 vol. 10×PBS after dissolved, and pH to 7.4 with Hcl). The fixative is good only for 3 weeks when stored in the dark at 4 C.
   f. Store in 70% ethanol. RNA FISH should be performed within 1-2 weeks.
3. For hybridization, the slides are dehydrated using 80%, 90%, 100% ethanol sequentially for 2 min. each,
4. Air dry slides at room temperature, <5 min.

Hybridization:
1. 4-10 μl preannealed probe DNA is pipetted onto each well of slide (amount depends on the surface area of the preparation).
2. Cover with clean 18 mm×18 mm (for small surface areas) cover slip or 18×50 mm (for larger spots) cover slip (VWR).
3. Optional: Seal edges of cover slip with rubber cement and incubate slide at 37° C. in a humid, dark chamber.
4. Hybridize in TC incubator.

Wash and Detection:
1. Each slide is washed three times for 5 minutes in 50% formamide, 2×SSC pH 7.4 at 45° C. with light agitation.
2. The slides are then washed 3 times for 5 minutes each at 45° C. in 2×SSC pH 7.4 with light agitation.
3. The slides are then blocked with 1% BSA (NEBL), 4×SSC, 0.1% Tween20 for 10 minutes at room temperature.
4. Detection: For biotinylated probes, 1:400 dilution of avidin-conjugated fluorochrome (Vector) is used; for DIG-labelled probes, 1:50 dilution of anti-digoxigenin antibody coupled to fluorochrome (Sigma, Fab fragments) is used. Dilutions are made in 1% BSA, 4×SSC, 0.1% Tween20. Incubate at 37° C. for 40 min.
5. The slides are then washed 3 times for 10 minutes each at 45° C. in 4×SSC pH 7.4, 0.1% (Tween20 with light agitation.
6. The slides are counterstained with DAPI for 5 minutes in 4×SSC and rinsed once for 1 min. in 4×SSC.
7. 3-10 μl Vectashield (Vector) mounting medium with antifade is applied to each well. Coverslips are sealed with nail polish.

RNA FISH Probes

Any oligonucleotide known in the art that specifically binds γ or β globin may be used for purposes of RNA FISH. See, e.g., Chakalova et al., 2005; Blood, 105:2154; and Gribnau et al., 2000; Cell; 5:377. The following oligonucleotide are specifically contemplated herein for use as RNA FISH probes:

| RNA FISH | |
|---|---|
| probe for | Sequence |
| γ globin | CGACCTGGACTTTTGCCAGGCACAGGGTCC |
| | [SEQ ID NO: 21] |
| | TCACTCCCAACCCCAGTATCTTCAAACAGC |
| | [SEQ ID NO: 22] |
| | GCATCTTTTTAACGACCATACTTGTCCTGC |
| | [SEQ ID NO: 23] |
| | ACAGAGCTGACTTTCAAAATCTACTCCAGC |
| | [SEQ ID NO: 24] |
| γ globin | Genbank accession number HUMBB |
| | 48910-50766, 52388-54776 |
| β globin | TTCCACACTGATGCAATCATTCGTCTGTTT |
| | [SEQ ID NO: 25] |
| | TGTGTACACATATTAAAACATTACACTTTA |
| | [SEQ ID NO: 26] |
| | ATTAGCAATATGAAACCTCTTACATCAGTT |
| | [SEQ ID NO: 27] |
| | AGTAATGTACTAGGCAGACTGTGTAAAGTT |
| | [SEQ ID NO: 28] |
| β globin | Genbank accession number HUMBB |
| | 58041-61921 |

In some embodiments, probes are selected for detecting mRNA but not genomic DNA. Such probes have the highest sequence difference between gamma and beta globin. Such probes include primers that span over intron(s) sequence so that only mRNA but not genomic DNA is detected.

Examples of oligonucleotide or PNA probes that can be used for RNA FISH of γ globin include: (1) CTG GAC TAG GAG CTT ATT GAT AAC CTC AGA CGT TC [SEQ ID NO: 29], and (2) GCA GAA TAA AGC CTA TCC TTG AAA GCT CTG AAT CAT [SEQ ID NO: 30]. Examples of oligonucleotide or PNA probes that can be used for RNA FISH of β globin include: (1) AGG GAA CAA AGG AAC CTT TAA TAG AAA TTG GAC AGC [SEQ ID NO: 31], and (2) TTT GAG GTT GCT AGT GAA CAC AGT TGT GT [SEQ ID NO: 32].

Examples of in vitro transcribed RNA probes that can be used for RNA FISH of γ globin include (1) CTG GAC TAG GAG CTT ATT GAT AAC CTC AGA CGT TC [SEQ ID NO: 33]; and (2) GCA GAA TAA AGC CTA TCC TTG AAA GCT CTG AAT CAT [SEQ ID NO: 34]. Examples of in vitro transcribed RNA probes that can be used for RNA FISH of β globin include (1) AGG GAA CAA AGG AAC CTT TAA TAG AAA TTG GAC AGC [SEQ ID NO: 35]; and (2) TTT GAG GTT GCT AGT GAA CAC AGT TGT GT [SEQ ID NO: 36].

In some embodiments, probes are selected for detecting nuclear transcript (e.g., nuclear RNA or nRNA). Preferably, such probes do not bind mRNA (e.g., intron/exon gap specific). Such probes can be selected to bind intron-exon junction(s) that have the highest difference in exon sequences.

Examples of oligonucleotide or PNA probes for γ globin include CCT GGT CAC CAG AGC CTA CCT TCC CAG GGT [SEQ ID NO: 37] and for β globin CTT GTA ACC TTG ATA CCA ACC TGC CCA G [SEQ ID NO: 38]. Examples of an in vitro transcribed RNA probe that can be used for RNA FISH of γ globin include CCT GGT CAC CAG AGC CTA CCT TCC CAG GGT [SEQ ID NO: 39], and for β globin CTT GTA ACC TTG ATA CCA ACC TGC CCA G [SEQ ID NO: 40].

In any of the embodiments herein, the RNA FISH probe is coupled to a molecular beacon for its detection. Examples of molecular beacons (labeling reagents) are known in the art and disclosed herein above.

### Example 2

### In Situ rt-PCR Identification of Fetal Nucleated Red Blood Cells from Maternal Nucleated Red Blood Cells in Solution

A highly purified cells population with fnRBCs and mnRBCs, and maternal white blood cells is fixed in 2% paraformaldehyde solution in 1×PBS for 20 minutes at room temperature, then the cells are collected by a brief centrifugation, and resuspended in 1×RT-PCR reaction buffer with dNTP mix, reverse transcriptase, Hot-start Taq DNA polymerase, fluorescence dye labeled primers for beta and gamma globin. This is followed by a reverse transcription reaction step at 50° C. for 30 minutes to convert the mRNA into cDNA and then followed by 30 to 40 cycles of PCR. The amplified products can be visualized under a fluorescent microscope and images may be captured through a COD camera. Cells demonstrating a high gamma to beta globin expression ratio will be selected and further genetic information will be extracted.

### Example 3

### In Situ rt-PCR Identification of Fetal Nucleated Red Blood Cell Nuclei from Maternal Nucleated Red Blood Cell Nuclei on Slide

A highly purified cells population with fnRBCs and mnRBCs, and maternal white blood cells is fixed in 2% paraformaldehyde solution in 1×PBS for 20 minutes at room temperature; the cells are then cytospin on to a slide. The cells are submerged in 1×rt-PCR reaction buffer with dNTP mix, reverse transcriptase, Hot-start Taq DNA polymerase, fluorescence dye labeled primers for beta and gamma globin. This is followed by a reverse transcription reaction step at 50° C. for 30 minutes to convert the mRNA into cDNA and then followed by 30 to 40 cycles of PCR. The amplified products can be visualized under a fluorescent microscope and images may be captured through a CCD camera. Cells demonstrating a high gamma to beta globin expression ratio will be selected and further genetic information will be extracted.

### Example 4

### In Situ rt-PCR Identification of Fetal Nucleated Red Blood Cell Nuclei from Maternal Nucleated Red Blood Cell Nuclei on Slide

A highly purified cells population with fnRBCs and mnRBCs, and maternal white blood cells is lysed with cold methanol: acetic acid (3:1) on ice for 20 minutes to release the nuclei. The nuclei are then cytospun on to a slide. The nuclei are submerged in 1×rt-PCR reaction buffer with dNTP mix, reverse transcriptase, Hot-start Taq DNA polymerase, fluorescence dye labeled primers for beta and gamma globin. This is followed by a reverse transcription reaction step at 50° C. for 30 minutes to convert the mRNA into cDNA and then followed by 30 to 40 cycles of PCR. The amplified products can be visualized under a fluorescent microscope and images may be captured through a CCD camera. Nuclei demonstrating a high gamma to beta globin expression ratio will be selected and further genetic information will be extracted.

### Example 5

### RNA FISH Identification of Fetal Nucleated Red Blood Cells from Maternal Nucleated Red Blood Cells on Slide

A highly purified cells population with fnRBCs and maternal nRBCs, and maternal white blood cells is lysed with cold methanol: acetic acid (3:1) on ice for 20 minutes; the nuclei are then cytospin on to a slide. As alternative, cells can be directly cytospin on to slides. Cells and nuclei are fixed and permeabilized with buffer well known to the art. The slide is hybridized with labeled probes, such as but not limited to probes for beta and gamma globin, over night at 37 C. The hybridization signal can be visualized under a fluorescent microscope and images may be captured through a CCD camera. The cells or nuclei demonstrating a high gamma to beta globin expression ratio will be selected and further genetic information will be extracted.

### Example 6

### RNA FISH Imaging of Fetal Nucleated Red Blood Cells or Nuclei from Maternal Nucleated Red Blood Cells or Nuclei on Slide

RNA FISH

Target cells (e.g., fnRBCs) or their nuclei are enriched by one or more methods disclosed herein or known in the art. Slides are prepared with target cells (fetal nRBCs or nuclei thereof) by cytospin methods. The slides with the target cells (or nuclei) are subjected to the procedure as listed above and probed with either of labeled oligonucleotide, PNA, in vitro transcribed RNA probes or nick translated DNA probes. The probes are hybridized overnight and the slides are washed according to the example above. Fetal cells or nuclei are imaged and detected using, e.g., a fluorescence microscopy.

Reverse Transcription and Polymerase Chain Reaction (RT-PCR)

Target cells (e.g., fnRBC's) or nuclei enriched by one or more of the methods disclosed herein or known in the art, that are either in solution or on slides, are first subjected to a paraformaldehyde fix, then a brief protease digestion. The target cells or nuclei are then subjected to in situ RT-PCR with labeled primers (e.g., those disclosed herein). These cells will go through few rounds of washing and the amplified products will be imaged using fluorescence microscopy.

Molecular Beacon or Hybridization Probes Detecting Amplified Products

RT-PCR primers that can amplify gamma and beta transcripts, e.g. forward primer TGG ACC CAG AGG TT TTT GA and reverse primer TGC AGC TTG TCA CAG TGC AG [SEQ ID NO: 41] are selected. Target cells (e.g., fetal nRBC's) or nuclei isolated using one or more of the methods disclosed herein or known in the art, either in solution or on slides, are first subjected to a paraformaldehyde fix, then a brief protease digestion. The target cells or nuclei are then subjected to in situ rt-PCR. Different fluorescent dye labeled molecular beacons targeting gamma and beta globin region with high sequence difference, such as gamma GAG CCA GGC CAT CAC TAA AGG CAC CG [SEQ ID NO: 42] and beta CTT TGT GGC ATC TCC CAA GGA AGT C[SEQ ID NO: 43], are allowed to hybridize with the amplification product in situ. Examples of fluorescent dyes are known in the art and disclosed herein above. The target cells or nuclei are then imaged under a fluorescence microscopy. Images may be captured through a COD camera. Alternatively, probes, such as γ probe GAG CCA GGC CAT CAC TAA AGG CAC CG [SEQ ID NO: 44] and β probe CTT TGT GGC ATC TCC CAA GGA AGT C [SEQ ID NO: 45], can be prepared either using PCR or in vitro transcription to incorporate biotin-11-dUTP or digoxingenin-16-dUTP, respectively. The modified probes are then allowed to hybridize with the amplified target in situ and then detect the target cells using antibody mediated imaging technology.

Reduction of Noise

In some embodiments, RT-PCR background can be reduced by selecting PCR primers that result in amplified products having a region of overlap. In this scenario, only the primers outside the overlap region is labeled.

In one embodiment, in situ PCR was performed for DYS1, a Y specific gene according to the following method: (1) Cells enriched by one or more of the methods disclosed herein (e.g., size-based separation followed by hemoglobin enrichment) are fixed with 2% paraformaldehyde at 20 minutes at room temperature. Then the cells are diluted 10× with 1×PBS, and are spun to remove unreacted paraformaldehyde; (2) The cells are then suspended in 1 mg/ml of protease K solution for 30 minutes, followed by 15 minutes at 98 C; (3) The fixed cells are subsequently used as input for in situ PCR or rt-PCR; (4) The cells are washed post-PCR with 1× with 2×SSC; (5) Target cells are detected using fluorescence microscopy.

### Example 7

### γ- and β-mRNA Expression in Fetal and Maternal nRBC Nuclei

Nuclei were prepared from enriched mnRBCs and buffy coat HE3 enriched cord blood using an acidic alcohol solution (methanol; acetic acid 3:1) to disrupt the cells and release the nuclei. RNA was isolated using Qiagen Rneasy mini kit. FIG. 9 below illustrates this embodiment. (FIG. 8)

### Example 8

### Gamma and Beta Expression in Single Nucleus

Nucleated RBCs from fetal cord blood were subjected to an acidic alcohol treatment (methanol: acetic acid 3:1) to disrupt the cells and release the nuclei. The resulting nuclei were washed once with 1×PBS, then diluted with 1×PBS to create single nucleus. The single nuclei were added directly into a real-time RT-PCR reaction. (FIG. 8)

### Example 9

### In Situ PCR for Detecting DYS1, Y specific Gene

Cells were isolated from male or female donor blood and fixed with 2% paraformaldehyde, then treated with 20 mg/ml of protease K and followed by 15 minutes 98 C. The cells were then added into a PCR reaction with Cy5 labeled PCR primers for Dys1. The Cy5 labeled PCR amplicons were detected using fluorescence microscopy. Male cells have a positive red signal and negative control female cells have a blue signal. The Figures below illustrate male cells in the first figure and female cells in the second figure.

### Example 10

### A Silicon Device Multiplexing 14 Three-Stage Array Duplexes

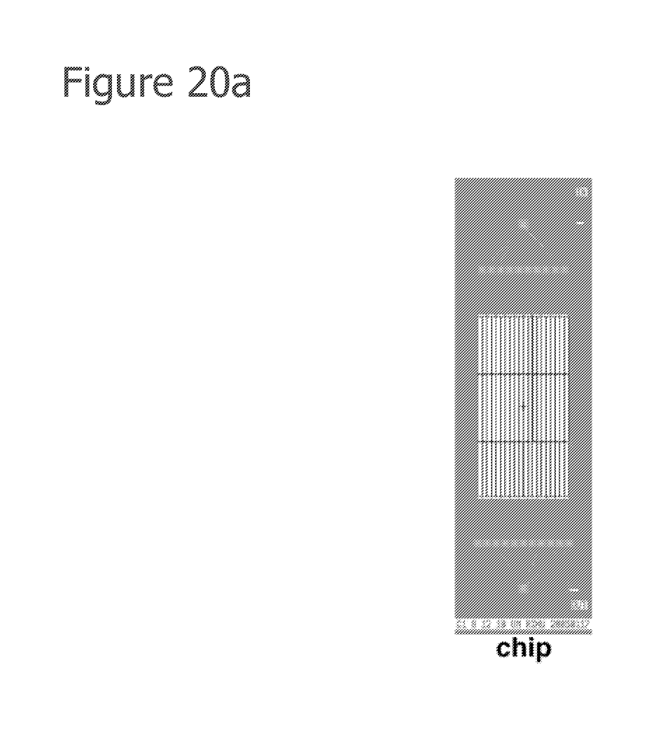

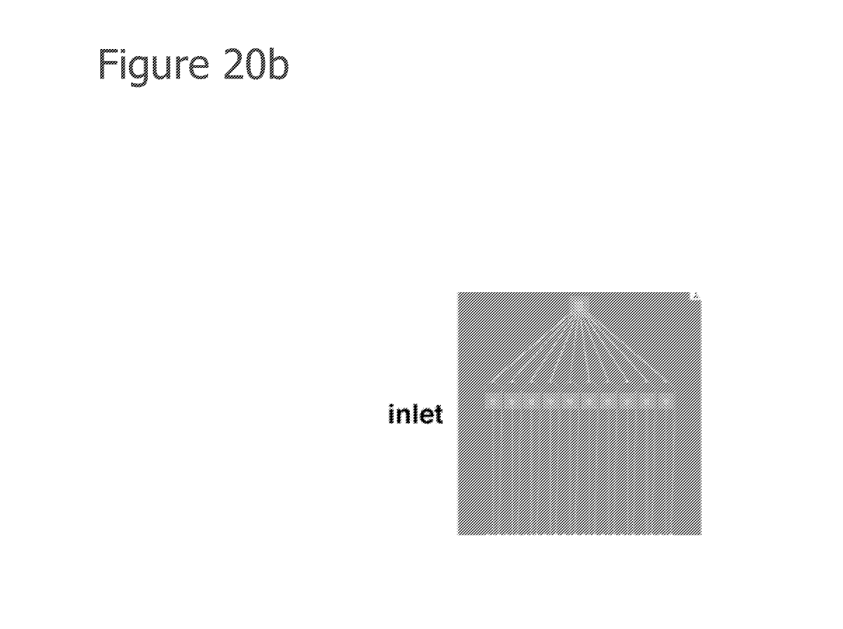

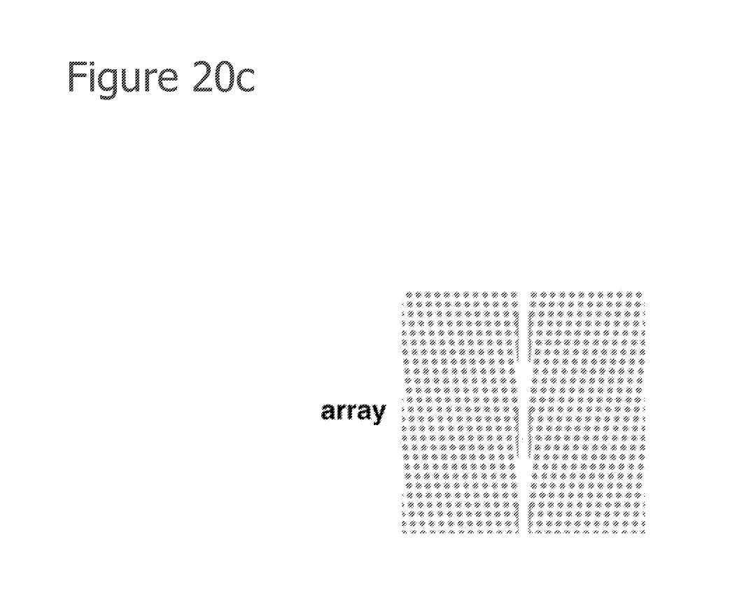

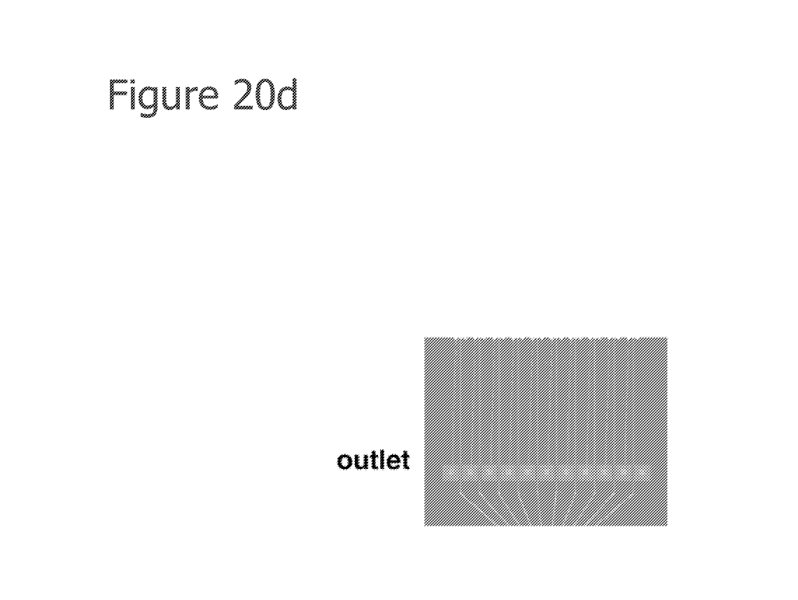

Exemplary size-based separation module (FIGS. 20A-D) of the invention, characterized as follows:

### Dimensions: 90 mm×34 mm×1 mm

Array design: 3 stages, gap size 18, 12 and 8 μm for the first, second and third stage, respectively. Bifurcation ratio=1/10.

### Duplex; single bypass channel

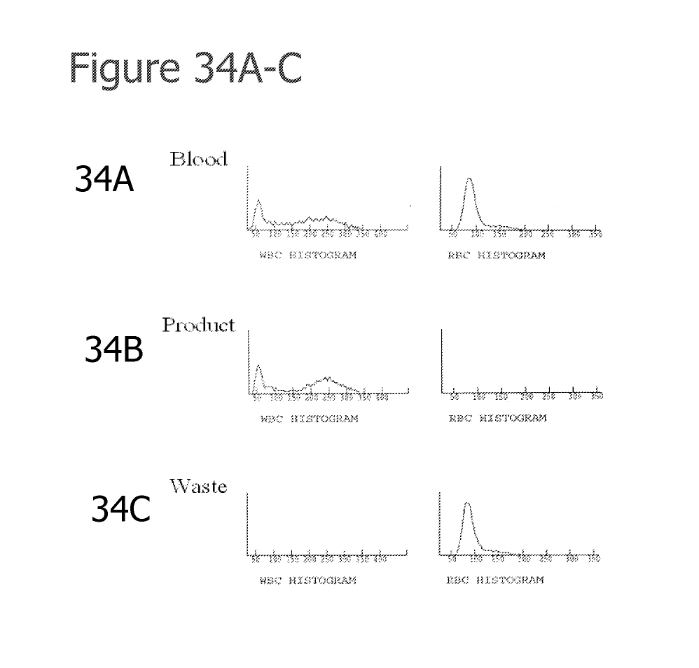

Device design: multiplexing 14 array duplexes; flow resistors for flow stability
Device fabrication: The arrays and channels were fabricated in silicon using standard photolithography and deep silicon reactive etching techniques. The etch depth is 150 μm. Through holes for fluid access are made using KOH wet etching. The silicon substrate was sealed on the etched face to form enclosed fluidic channels using a blood compatible pressure sensitive adhesive (9795, 3M, St Paul. Minn.).
Device packaging: The device was mechanically mated to a plastic manifold with external fluidic reservoirs to deliver blood and buffer to the device and extract the generated fractions.
Device operation: An external pressure source was used to apply a pressure of 2.4 PSI to the buffer and blood reservoirs to modulate fluidic delivery and extraction from the packaged device.
Experimental conditions: human blood from consenting adult donors was collected into K₂EDTA vacutainers (366643, Becton Dickinson, Franklin Lakes, N.J.). The undiluted blood was processed using the exemplary device described above at room temperature and within 9 brs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100 ML, Sigma-Aldrich, St Louis, Mo.).
Measurement techniques: Complete blood counts were determined using a Coulter impedance hematology analyzer (COULTER Ac•T diff, Beckman Coulter, Fullerton, Calif.).
Performance: FIGS. 34A-F shows typical histograms generated by the hematology analyzer from a blood sample and the waste (buffer, plasma, red blood cells, and platelets) and product (buffer and nucleated cells) fractions generated by the device. The following table shows the performance over 5 different blood samples:

| | | Performance Metrics | | |
|---|---|---|---|---|
| a) Sample | | RBC | Platelet | |
| number | i) throughput | removal | removal | WBC loss |
| 1 | 4 mL/hr | 100% | 99% | <1% |
| 2 | 6 mL/hr | 100% | 99% | <1% |
| 3 | 6 mL/hr | 100% | 99% | <1% |
| 4 | 6 mL/hr | 100% | 97% | <1% |
| 5 | 6 mL/hr | 100% | 98% | <1% |

### Example 11

### A Silicon Device Multiplexing 14 Single-Stage Array Duplexes

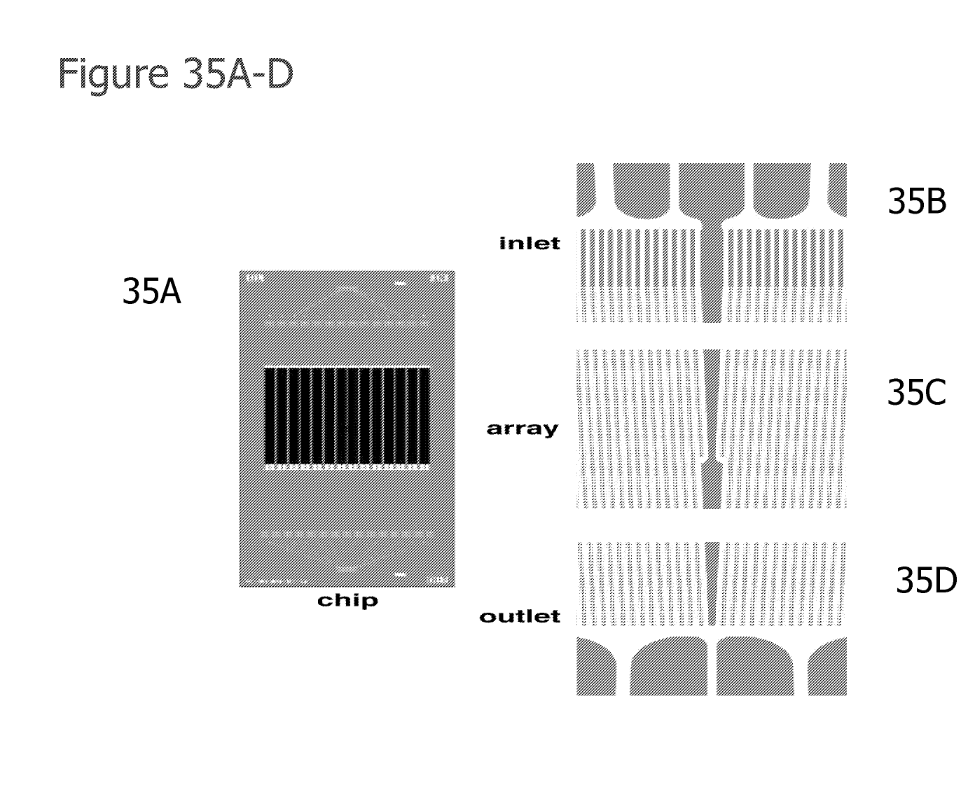

### FIGS. 35A-D show an exemplary device of the invention, characterized as follows.

### Dimensions: 90 mm×34 mm×1 mm

Array design: 1 stage, gap size=24 μm. Bifurcation ratio=1/60. Duplex; double bypass channel
Device design: multiplexing 14 array duplexes; flow resistors for flow stability
Device fabrication: The arrays and channels were fabricated in silicon using standard photolithography and deep silicon reactive etching techniques. The etch depth is 150 μm. Through holes for fluid access are made using KOH wet etching. The silicon substrate was sealed on the etched face to form enclosed fluidic channels using a blood compatible pressure sensitive adhesive (9795, 3M, St Paul, Minn.).
Device packaging: The device was mechanically mated to a plastic manifold with external fluidic reservoirs to deliver blood and buffer to the device and extract the generated fractions.
Device operation: An external pressure source was used to apply a pressure of 2.4 PSI to the buffer and blood reservoirs to modulate fluidic delivery and extraction from the packaged device.
Experimental conditions: human blood from consenting adult donors was collected into K₂EDTA vacutainers (366643, Becton Dickinson, Franklin Lakes, N.J.). The undiluted blood was processed using the exemplary device described above at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100 ML, Sigma-Aldrich, St Louis, Mo.).
Measurement techniques: Complete blood counts were determined using a Coulter impedance hematology analyzer (COULTER Ac•T diff, Beckman Coulter. Fullerton, Calif.).

### Performance: The device operated at 17 mL/hr and achieved >99% red blood cell removal, >95% nucleated cell retention, and >98% platelet removal.

### Example 12

### Separation of Fetal Cord Blood

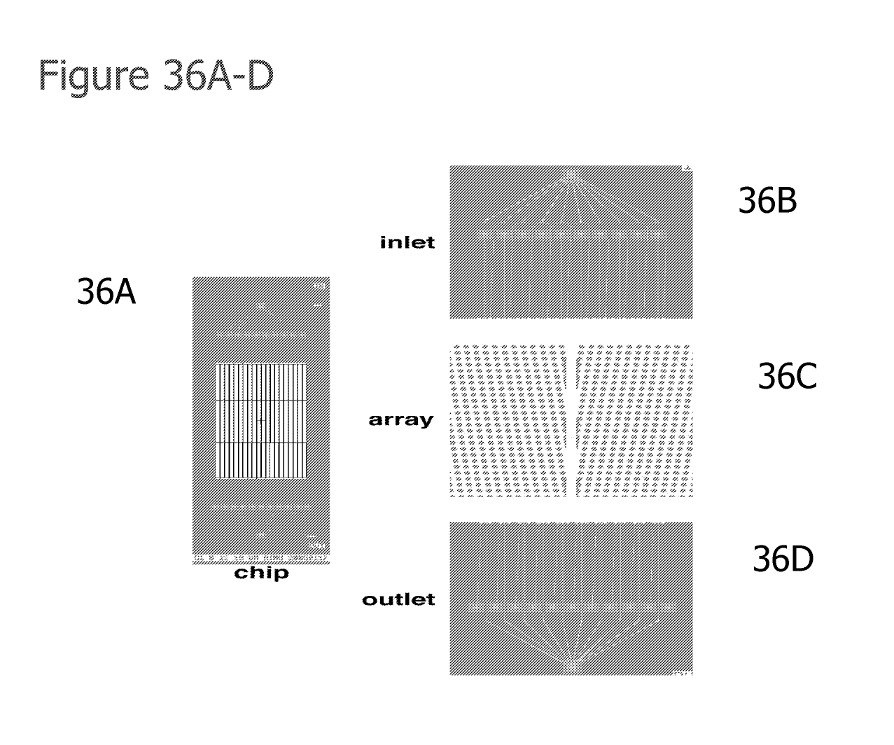

### FIGS. 36A-D show a schematic of the device used to separate nucleated cells from fetal cord blood.

### Dimensions: 100 mm×28 mm×1 mm

Array design: 3 stages, gap size=18, 12 and 8 μm for the first, second and third stage, respectively. Bifurcation ratio=1/10.

### Duplex; single bypass channel.

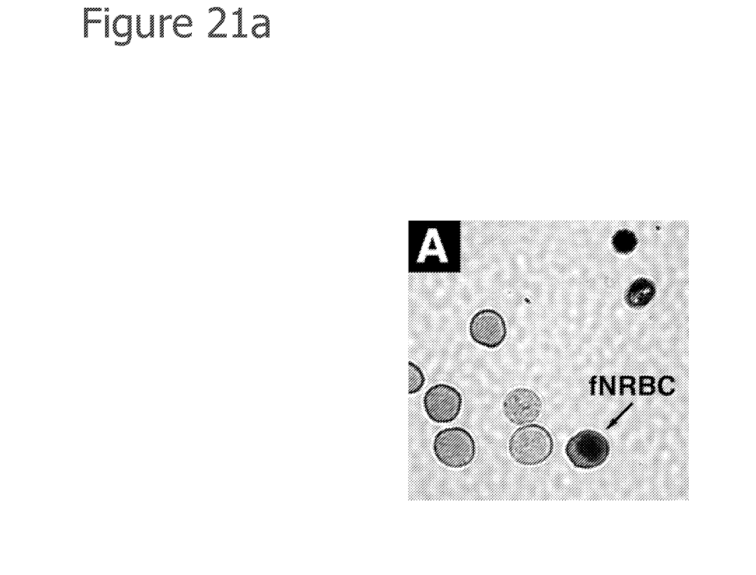

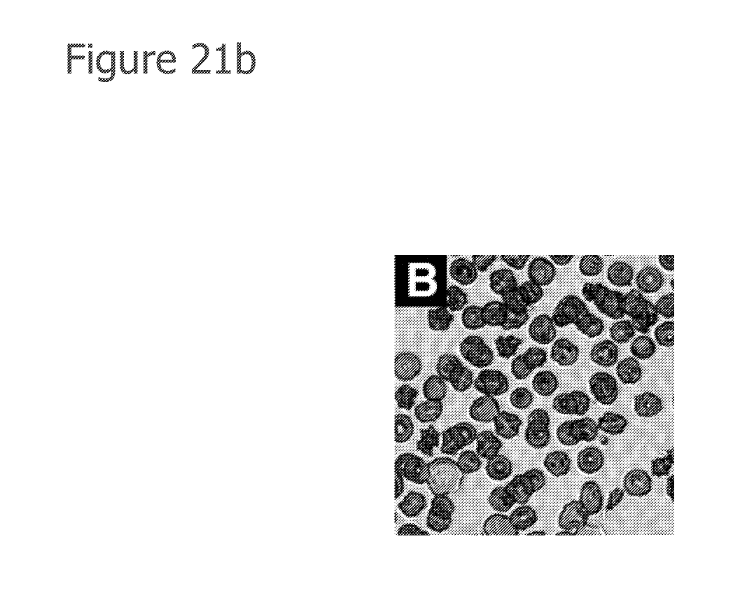

Device design: multiplexing 1.0 array duplexes; flow resistors for flow stability.
Device fabrication: The arrays and channels were fabricated in silicon using standard photolithography and deep silicon reactive etching techniques. The etch depth is 140 μm. Through holes for fluid access are made using KOH wet etching. The silicon substrate was sealed on the etched face to form enclosed fluidic channels using a blood compatible pressure sensitive adhesive (9795, 3M, St Paul, Minn.).
Device packaging: The device was mechanically mated to a plastic manifold with external fluidic reservoirs to deliver blood and buffer to the device and extract the generated fractions.
Device operation: An external pressure source was used to apply a pressure of 2.0 PSI to the buffer and blood reservoirs to modulate fluidic delivery and extraction from the packaged device.
Experimental conditions: Human fetal cord blood was drawn into phosphate buffered saline containing Acid Citrate Dextrose anticoagulants. One mL of blood was processed at 3 mL/hr using the device described above at room temperature and within 48 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen. Carlsbad, Calif.) containing 1% Bovine Serum Albumin (BSA) (A8412-100 ML, Sigma-Aldrich, St Louis, Mo.) and 2 mM EDTA (15575-020, Invitrogen, Carlsbad, Calif.).
Measurement techniques: Cell smears of the product and waste fractions (FIG. 21A-B) were prepared and stained with modified Wright-Giemsa (WG16, Sigma Aldrich, St. Louis, Mo.).

### Performance: Fetal nucleated red blood cells were observed in the product fraction (FIG. 21A) and absent from the waste fraction (FIG. 21B).

### Example 13

### Isolation of Fetal Cells from Maternal Blood

### The device and process described in detail in Example 1 were used in combination with immunomagnetic affinity enrichment techniques to demonstrate the feasibility of isolating fetal cells from maternal blood.

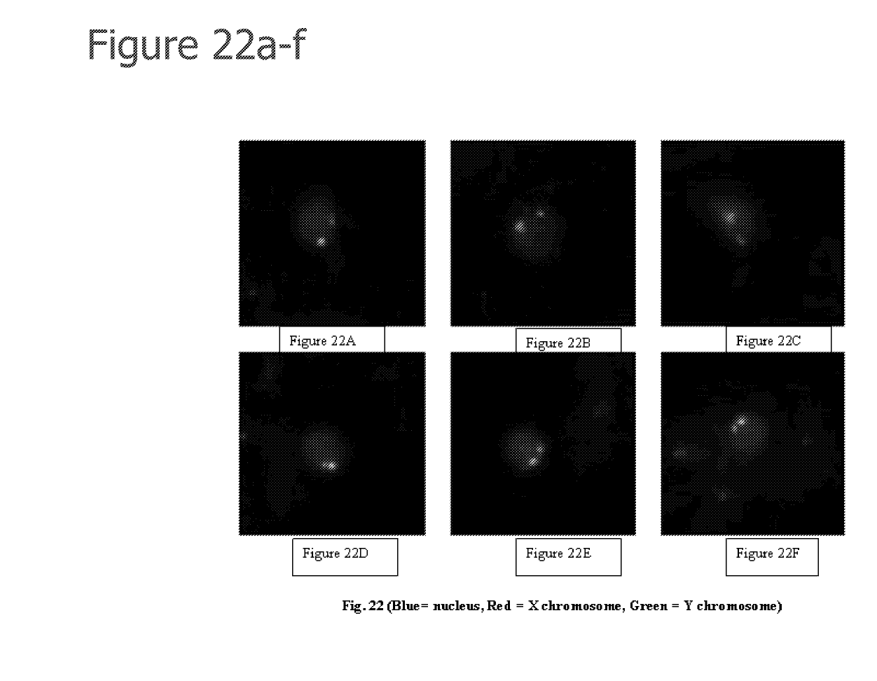

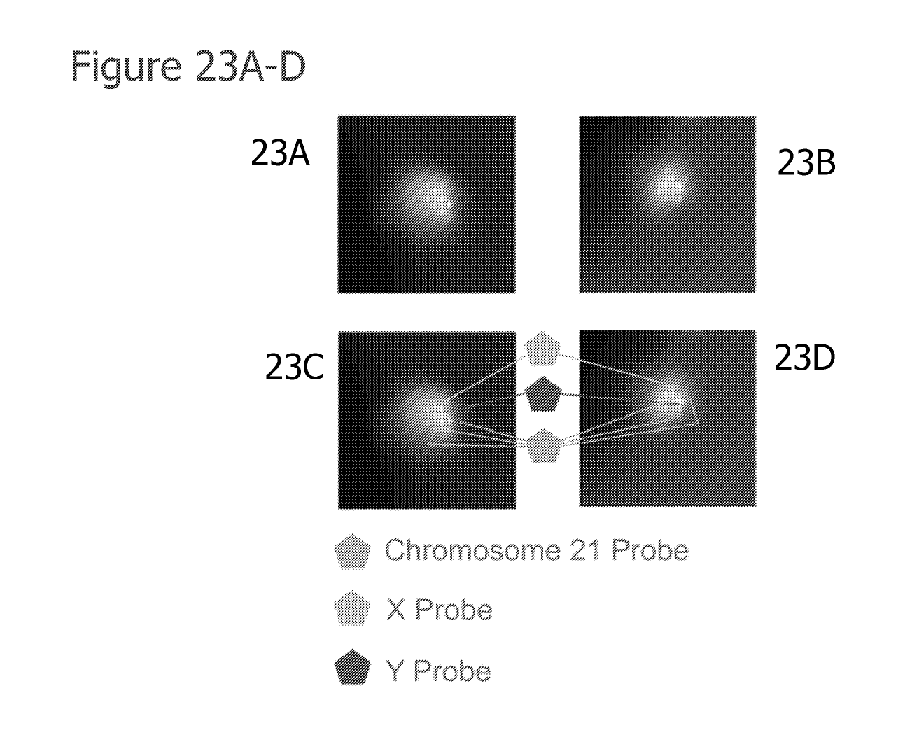

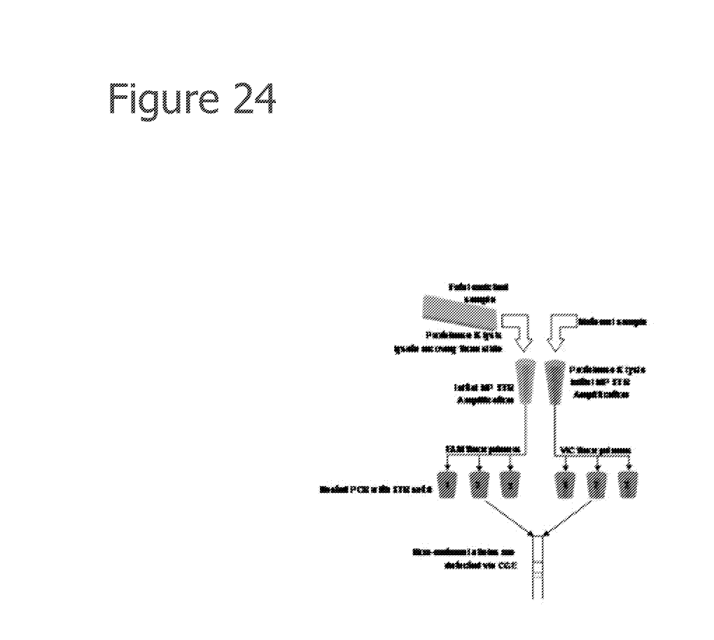

Experimental conditions: blood from consenting maternal donors carrying male fetuses was collected into K₂EDTA vacutainers (366643, Becton Dickinson. Franklin Lakes, N.J.) immediately following elective termination of pregnancy. The undiluted blood was processed using the device described in Example 1 at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100 ML, Sigma-Aldrich, St Louis, Mo.). Subsequently, the nucleated cell fraction was labeled with anti-CD71 microbeads (130-046-201, Miltenyi Biotech Inc., Auburn, Calif.) and enriched using the MiniMACS™ MS column (130-042-201, Miltenyi Biotech Inc., Auburn, Calif.) according to the manufacturer's specifications. Finally, the CD71-positive fraction was spotted onto glass slides.
Measurement techniques: Spotted slides were stained using fluorescence in situ hybridization (FISH) techniques according to the manufacturer's specifications using Vysis probes (Abbott Laboratories, Downer's Grove, Ill.). Samples were stained from the presence of X and Y chromosomes. In one case, a sample prepared from a known Trisomy 21 pregnancy was also stained for chromosome 21.
Performance: Isolation of fetal cells was confirmed by the reliable presence of male cells in the CD71-positive population prepared from the nucleated cell fractions (FIG. 22). In the single abnormal case tested, the trisomy 21 pathology was also identified (FIG. 23).
The following examples show specific embodiments of devices of the invention. The description for each device provides the number of stages in series, the gap size for each stage, ε (Flow Angle), and the number of channels per device (Arrays/Chip). Each device was fabricated out of silicon using DRIE, and each device had a thermal oxide layer.

### Example 14

### This device includes five stages in a single array.

| Array Design: | 5 stage, asymmetric array | |
|---|---|---|
| Gap Sizes: | Stage 1: | 8 μm |
| | Stage 2: | 10 μm |
| | Stage 3: | 12 μm |
| | Stage 4: | 14 μm |
| | Stage 5: | 16 μm |

| | |
|---|---|
| Flow Angle: | 1/10 |
| Arrays/Chip: | 1 |

### Example 15

This device includes the stages, where each stage is a duplex having a bypass channel. The height of the device was 125 μm.

| Array Design: | symmetric 3 stage array | |
|---|---|---|
| | with central collection | |
| | channel | |
| Gap Sizes: | Stage 1: | 8 μm |
| | Stage 2: | 12 μm |
| | Stage 3: | 18 μm |

| | |
|---|---|
| Flow Angle: | 1/10 |
| Arrays/Chip: | 1 |
| Other | central collection channel |

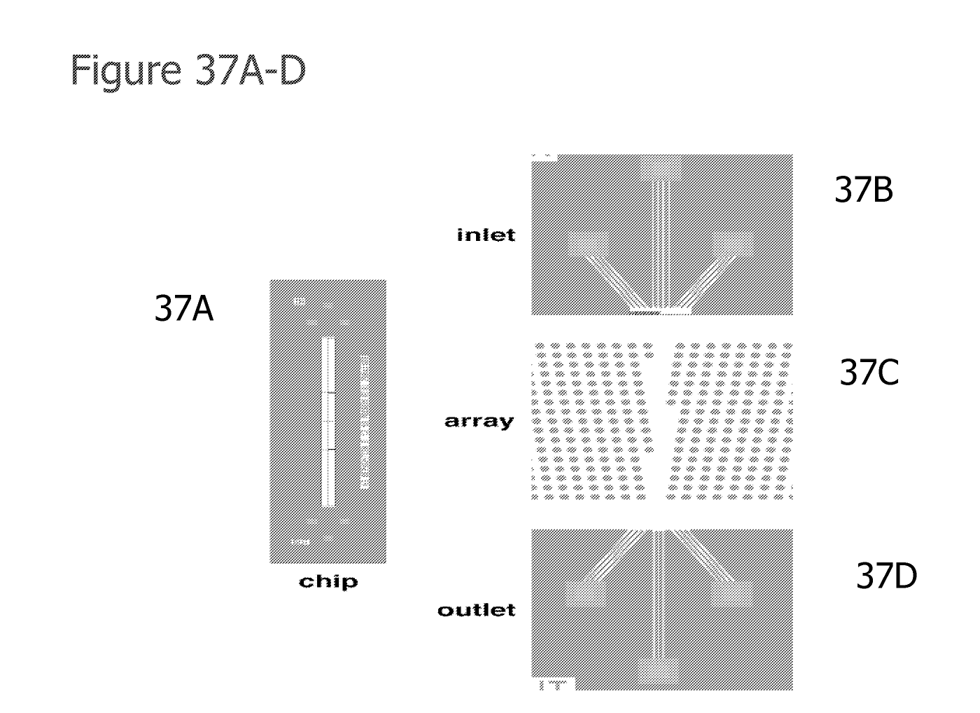

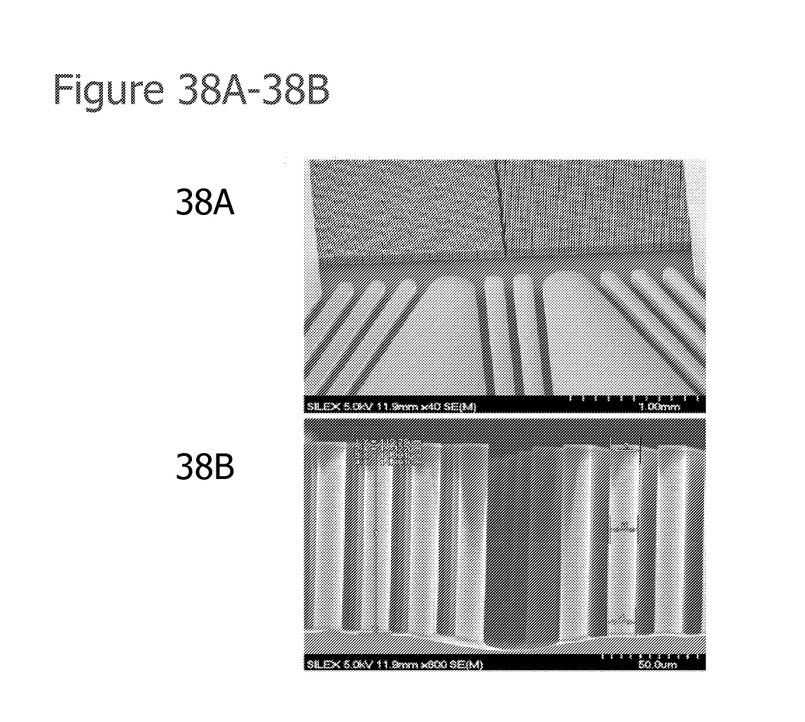

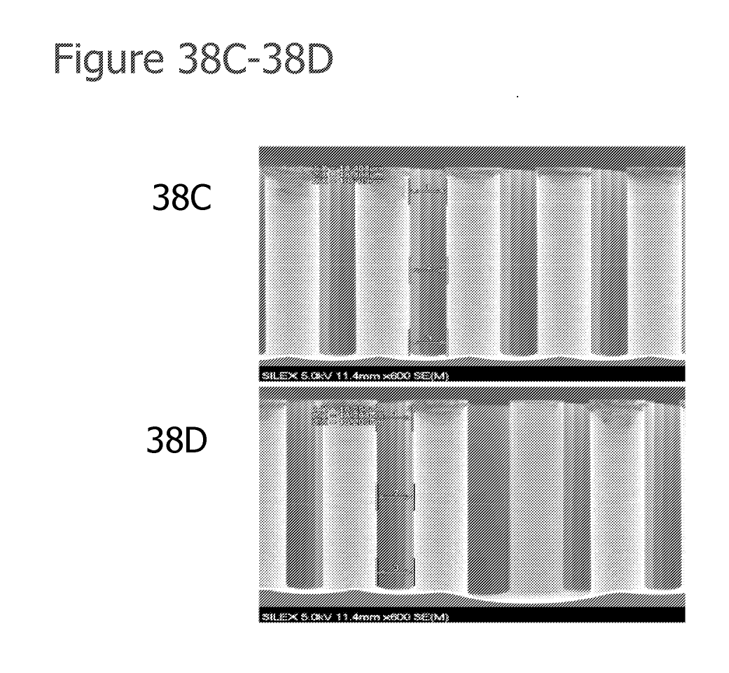

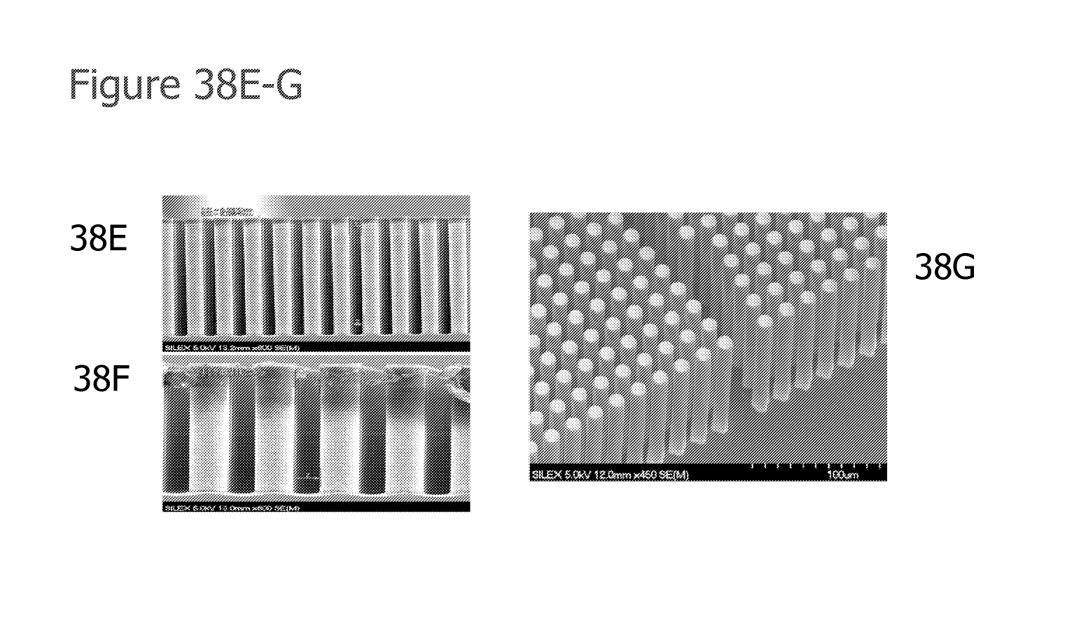

FIG. 37A shows the mask employed to fabricate a size-based separation device herein. FIGS. 37B-D are enlargements of the portions of the mask that define the inlet, array, and outlet. FIGS. 38A-G show SEMs of a size based separation module herein.

### Example 16

This device includes the stages, where each stage is a duplex having a bypass channel, “Fins” were designed to flank the bypass channel to keep fluid from the bypass channel from re-entering the array. The chip also included on-chip flow resistors, i.e., the inlets and outlets possessed greater fluidic resistance than the array. The height of the device was 117 μm.

| Array Design: | 3 stage symmetric array | |
|---|---|---|
| Gap Sizes: | Stage 1: | 8 μm |
| | Stage 2: | 12 μm |
| | Stage 3: | 18 μm |

| | |
|---|---|
| Flow Angle: | 1/10 |
| Arrays/Chip: | 10 |
| Other | large fin central collection channel |
| | on-chip flow resistors |

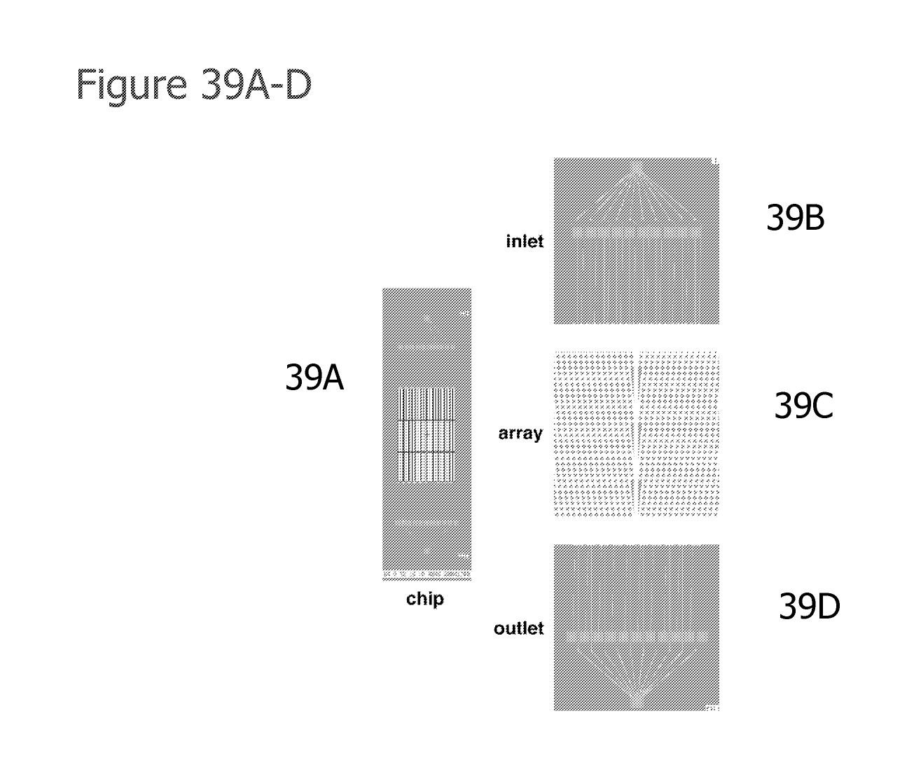

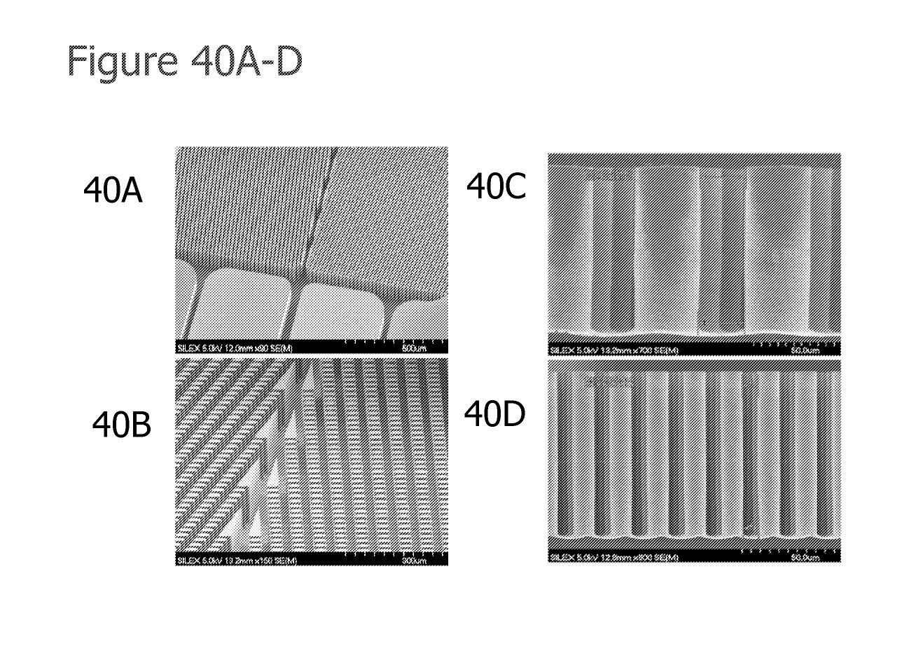

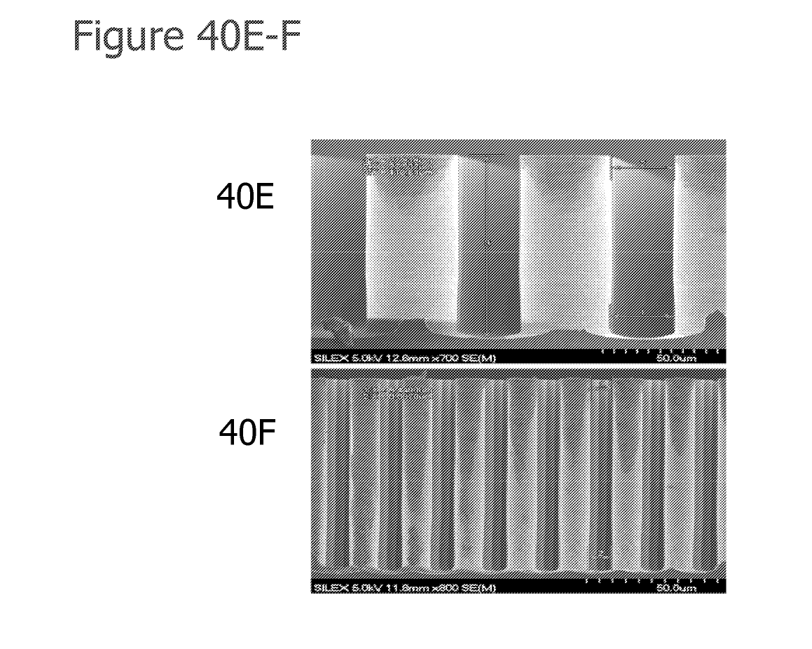

FIG. 39A shows the mask employed to fabricate a size-based separation module herein. FIGS. 39B-39D are enlargements of the portions of the mask that define the inlet, array, and outlet. FIGS. 40A-40F show SEMs of a separation module used in this example.

### Example 17

This device includes the stages, where each stage is a duplex having a bypass channel. “Fins” were designed to flank the bypass channel to keep fluid from the bypass channel from re-entering the array. The edge of the fin closest to the array was designed to mimic the shape of the array. The chip also included on-chip flow resistors, i.e., the inlets and outlets possessed greater fluidic resistance than the array. The height of the device was 138 μm.

| Array Design: | 3 stage symmetric array | |
|---|---|---|
| Gap Sizes: | Stage 1: | 8 μm |
| | Stage 2: | 12 μm |
| | Stage 3: | 18 μm |

| | |
|---|---|
| Flow Angle: | 1/10 |
| Arrays/Chip: | 10 |
| Other | alternate large fin central collection channel |
| | on-chip flow resistors |

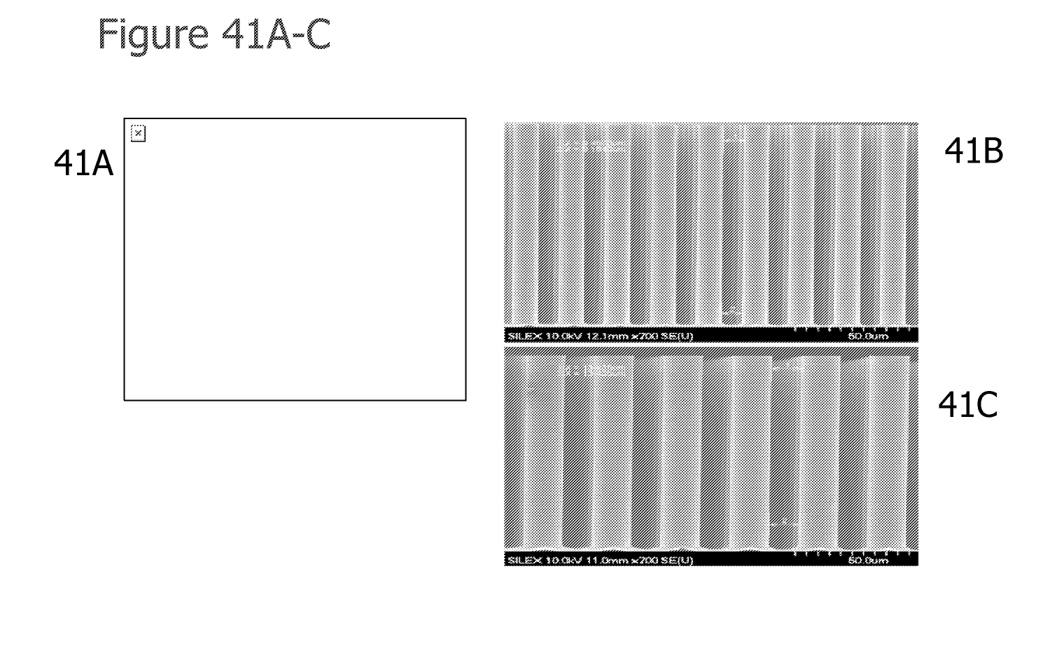

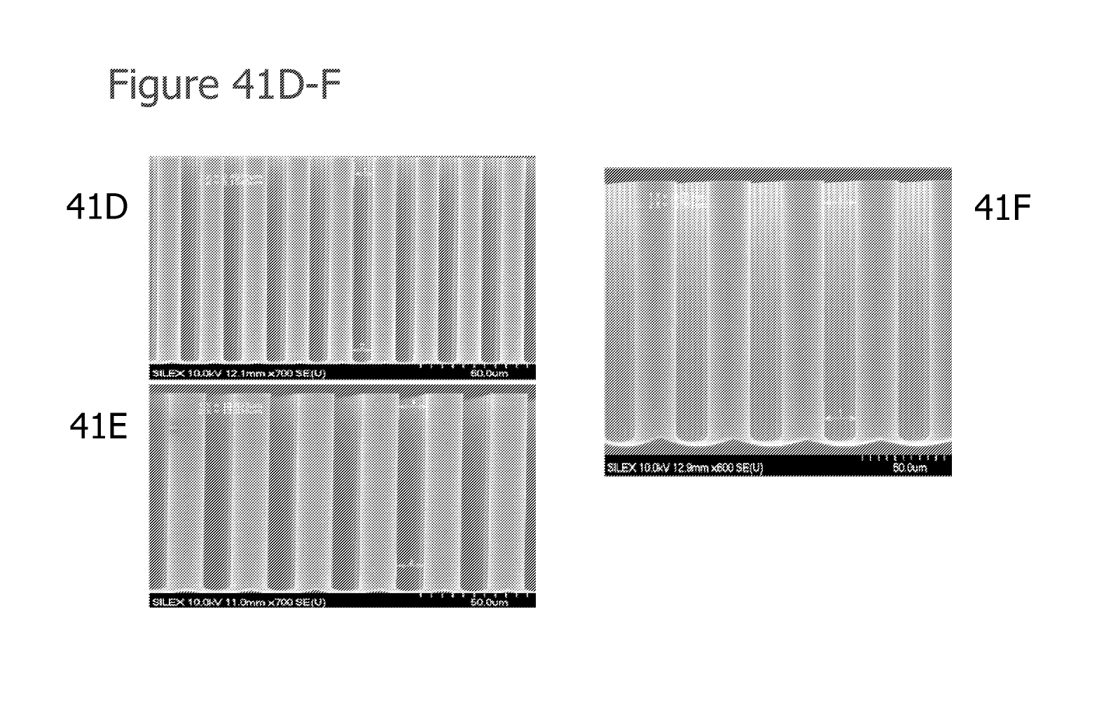

FIG. 36A shows the mask employed to fabricate the device. FIGS. 36B-36D are enlargements of the portions of the mask that define the inlet, array, and outlet. FIGS. 41A-41F show SEMs of a device as described above.

### Example 18

This device includes the stages, where each stage is a duplex having a bypass channel. “Fins” were optimized to flank the bypass channel to keep fluid from the bypass channel from re-entering the array. The edge of the fin closest to the array was designed to mimic the shape of the array. The chip also included on-chip flow resistors. i.e., the inlets and outlets possessed greater fluidic resistance than the array. The height of the device was 139 or 142 μm.

| Array Design: | 3 stage symmetric array | |
|---|---|---|
| Gap Sizes: | Stage 1: | 8 μm |
| | Stage 2: | 12 μm |
| | Stage 3: | 18 μm |

| | |
|---|---|
| Flow Angle: | 1/10 |
| Arrays/Chip: | 10 |
| Other | Femlab optimized central collection channel (Femlab I) |
| | on-chip flow resistors |

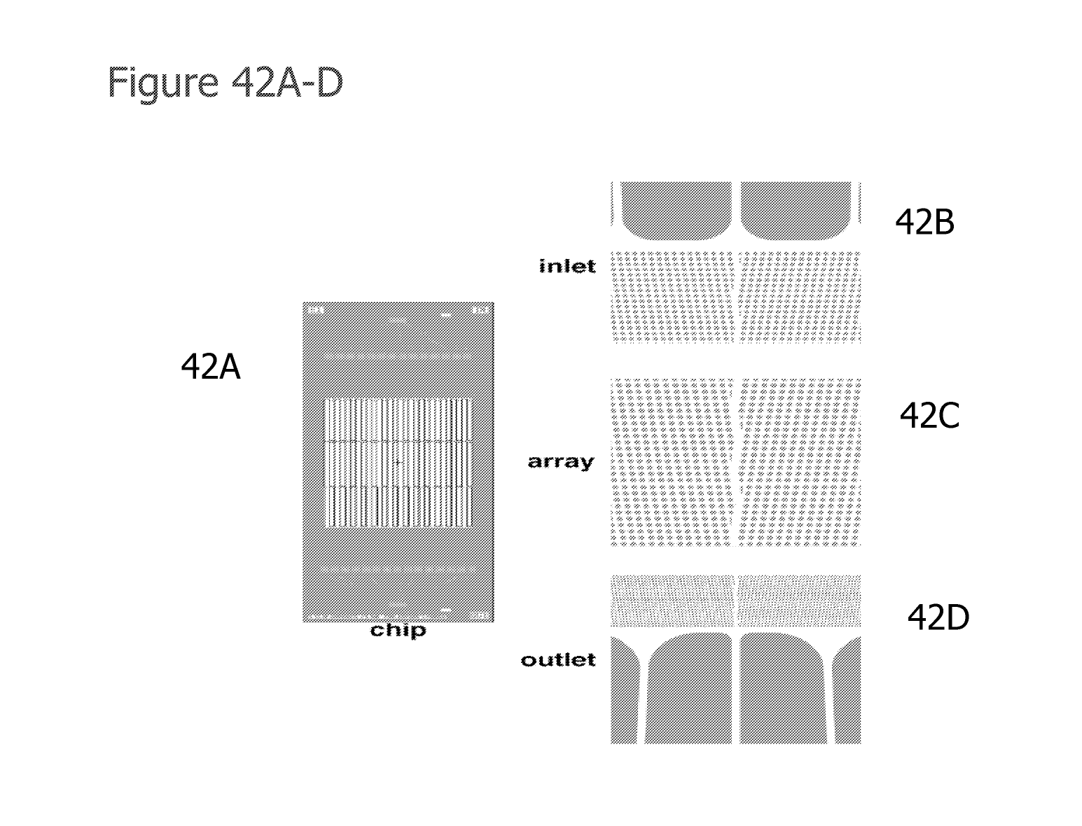

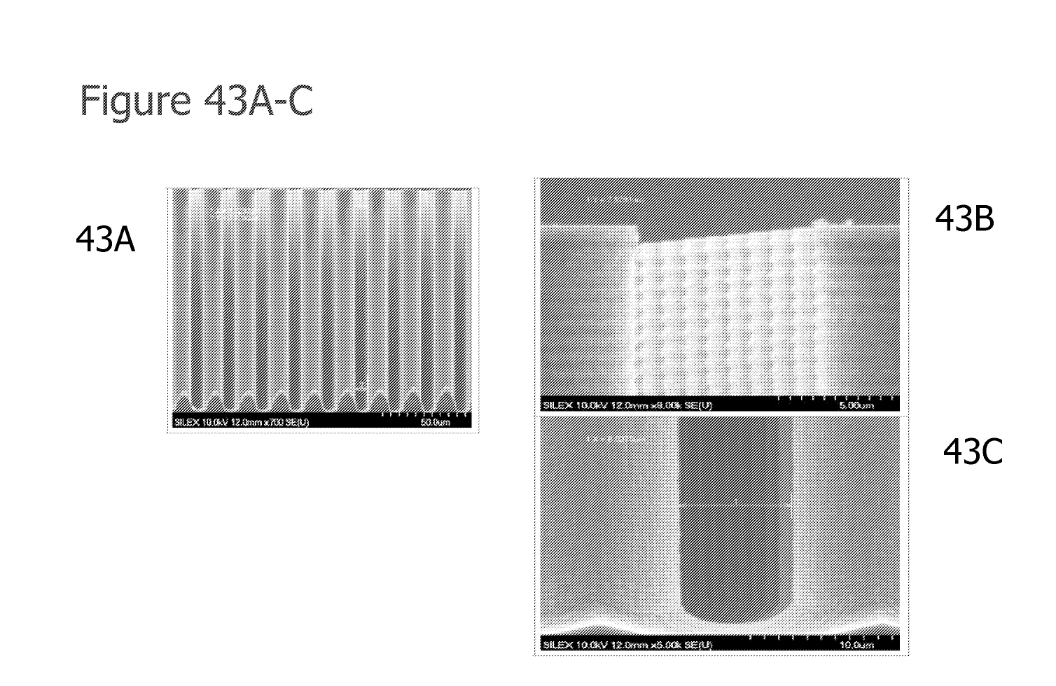

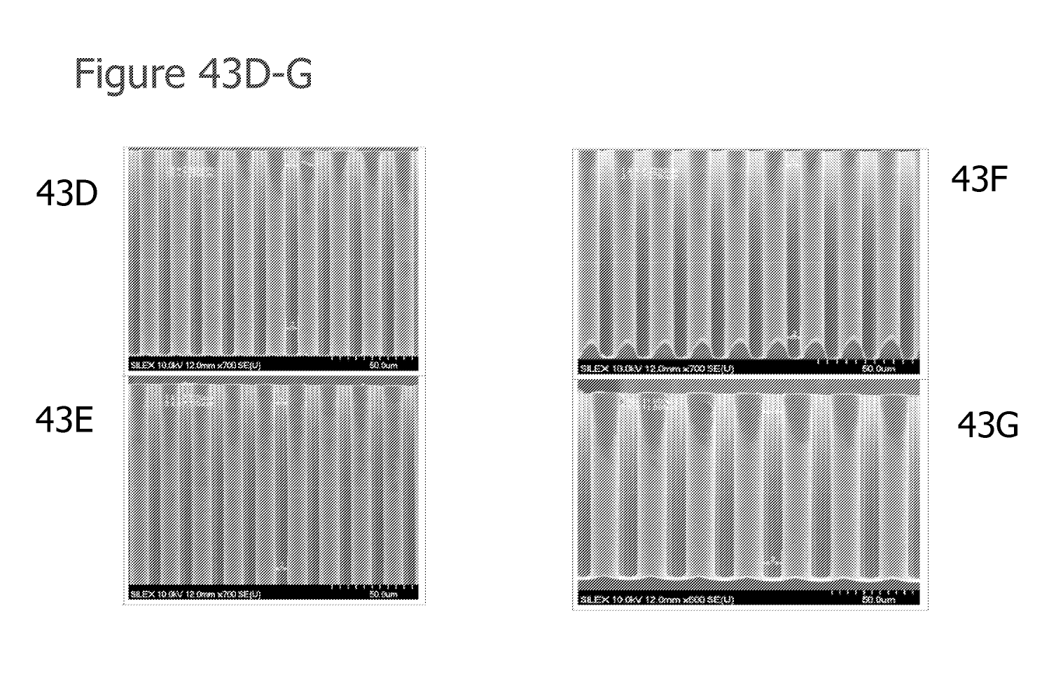

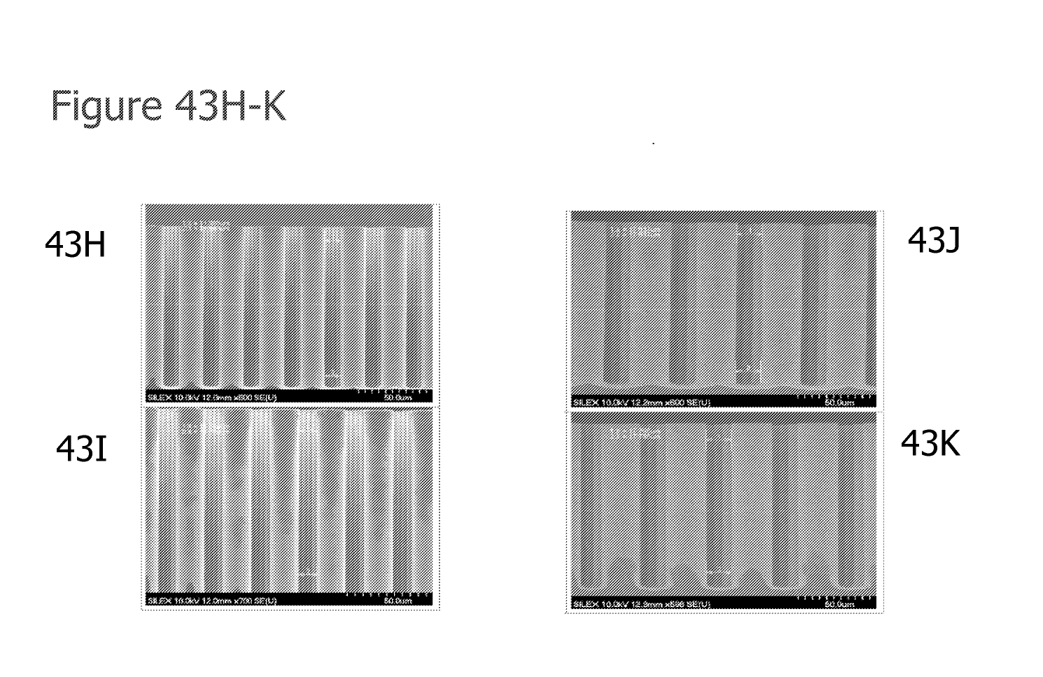

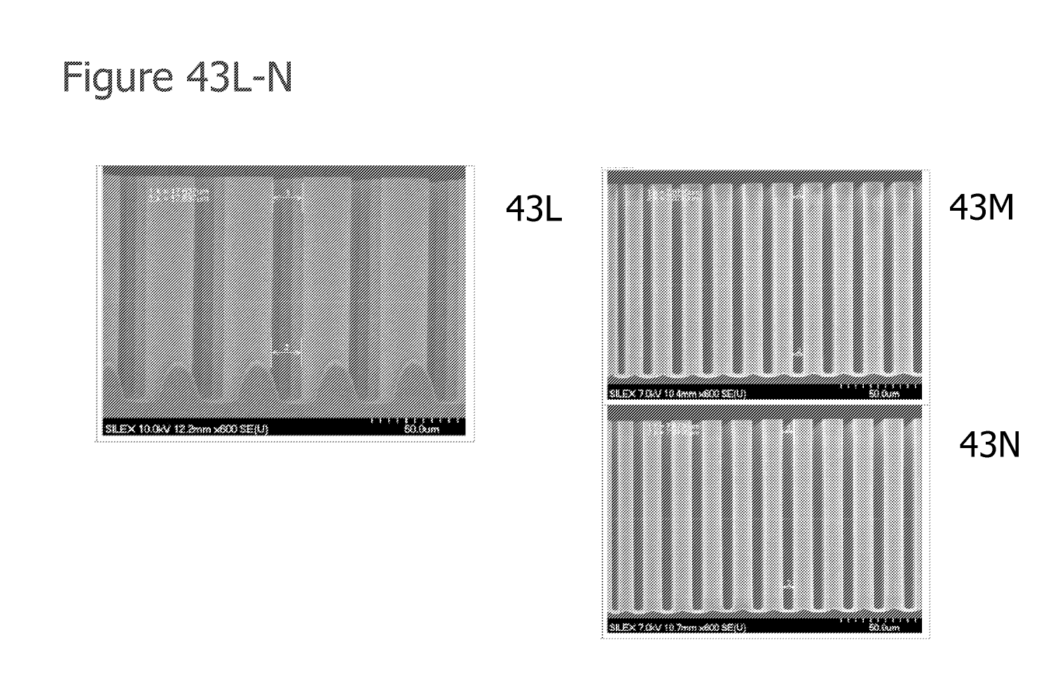

FIG. 42A shows the mask employed to fabricate the device. FIGS. 42B-42D are enlargements of the portions of the mask that define the inlet, array, and outlet. FIGS. 43A-43S show SEMs of the above device.

### Example 19

This device includes a single stage, duplex device having a bypass channel disposed to receive output from the ends of both arrays. The obstacles in this device are elliptical. The chip also included on-chip flow resistors, i.e., the inlets and outlets possessed greater fluidic resistance than the array. The height of the device was 152 μm.

| Array Design: | singie stage symmetric array | |
|---|---|---|
| Gap Sizes: | Stage 1: | 24 μm |

| | |
|---|---|
| Flow Angle: | 1/60 |
| Arrays/Chip: | 14 |
| Other | central barrier |
| | ellipsoid posts |
| | on-chip resistors |
| | Femlab modeled array |
| | boundary |

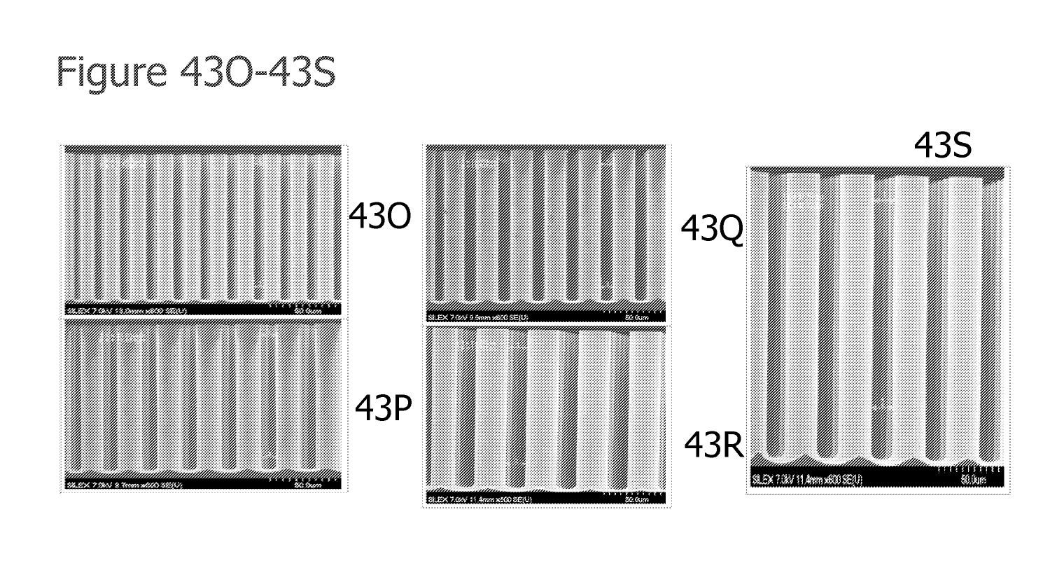

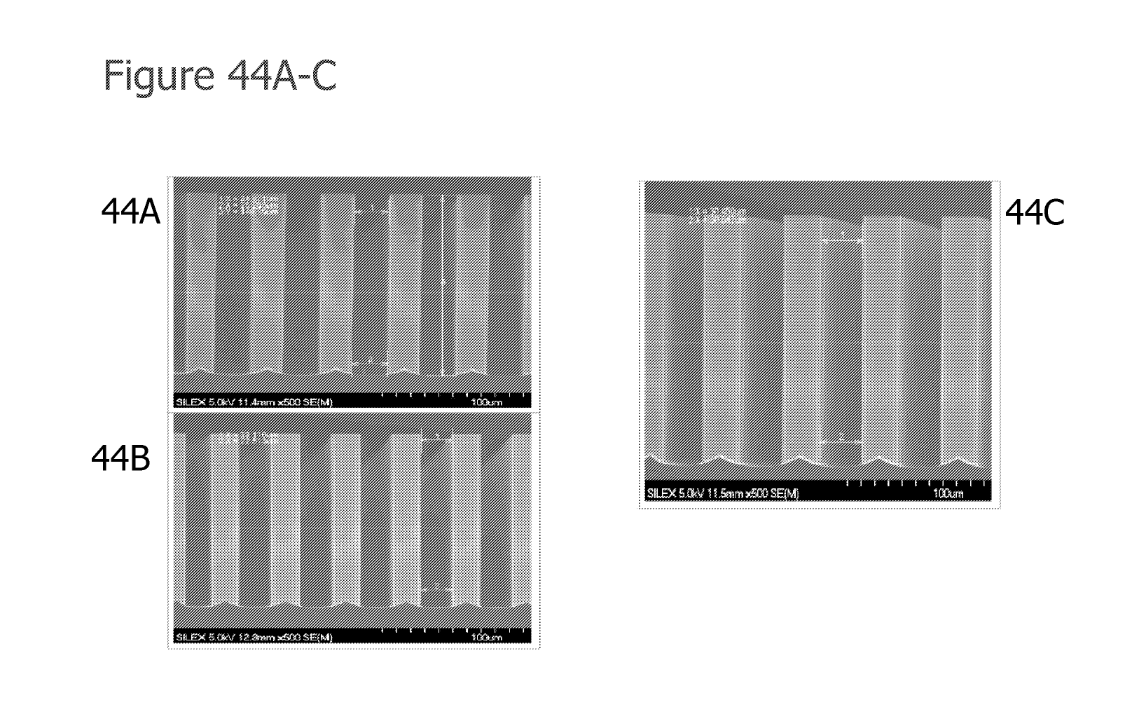

FIG. 35A shows the mask employed to fabricate the device. FIGS. 35B-35D are enlargements of the portions of the mask that define the inlet, array, and outlet. FIGS. 44A-44C show SEMs of the actual device.

### Example 20

### Comparative Genomic Hybridization (CGH) for Aneuploidy

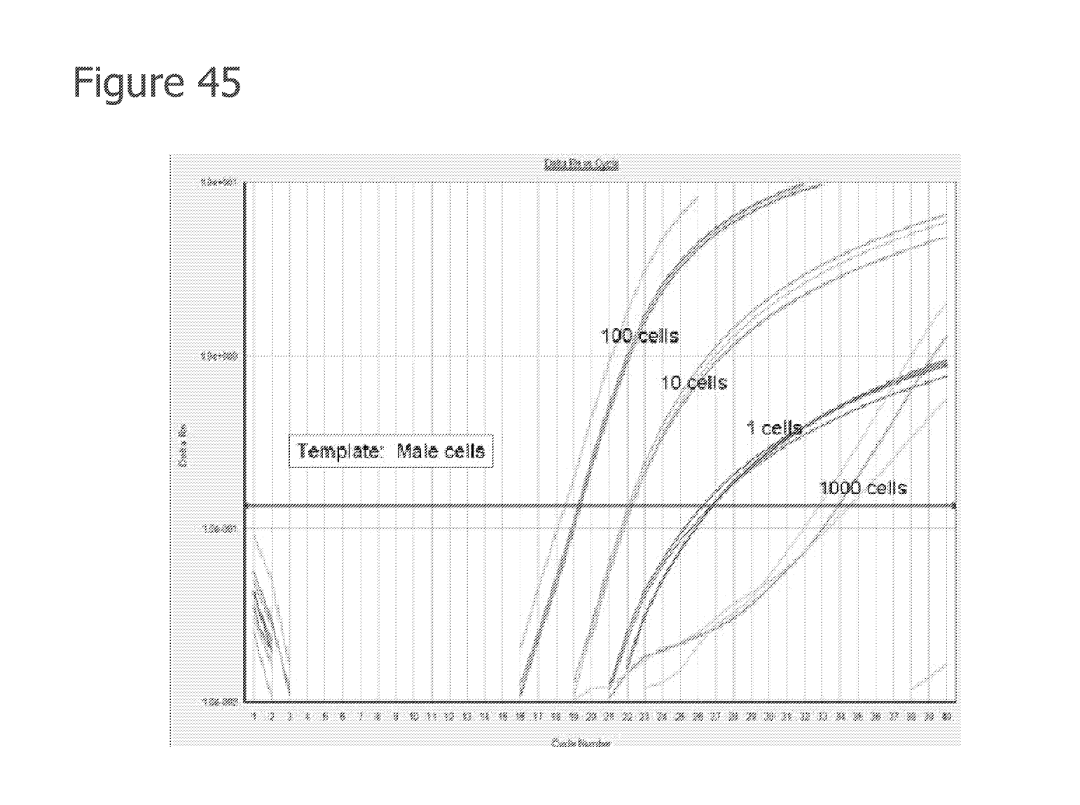

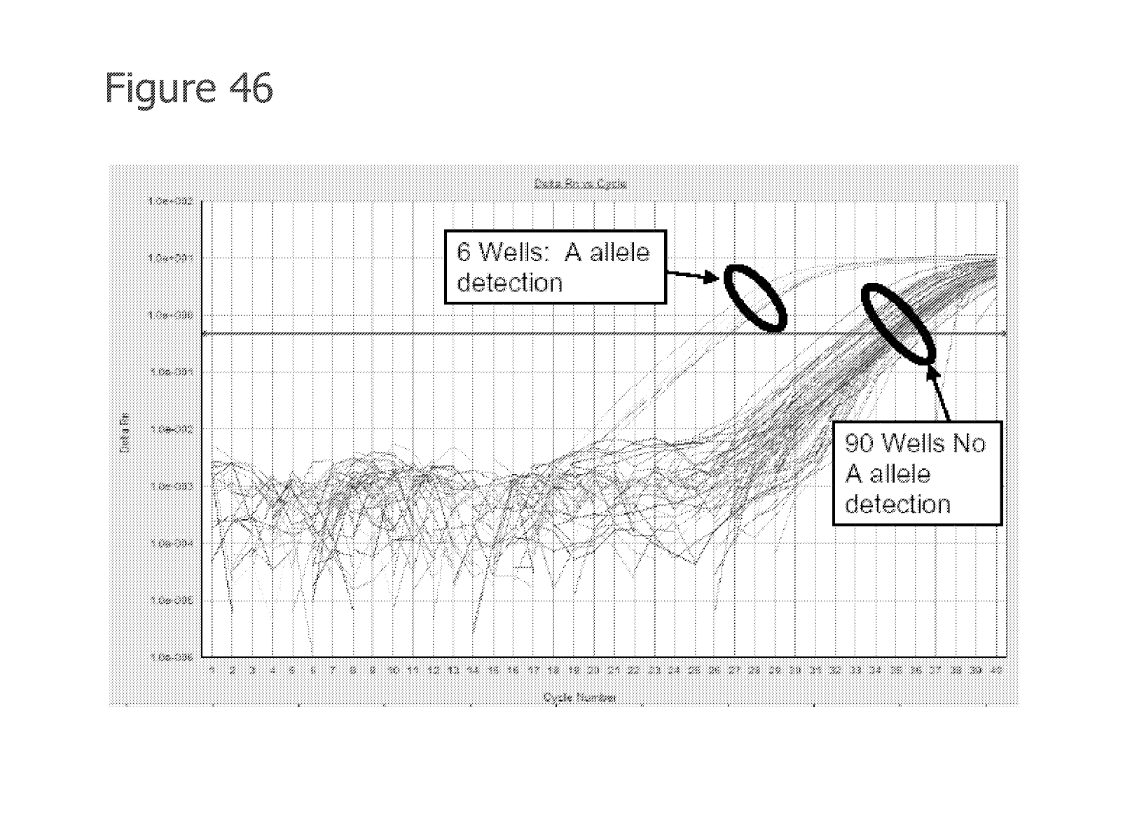

Agilent Technologies commercial human CGH array and whole genome amplification procedure (based on multiple displacement amplification) were used to demonstrate the ability to detect aneuploidy in target cells resident in cell mixtures. The test sample was simulated with genomic DNA from a cell line with a triple-X chromosome, and the control sample was DNA from a normal (diploid-X) cell line. Differential (2-color) hybridization was performed with amplification products from: (1) the control DNA and (2) a mixture of 70% control DNA and 30% triple-X DNA. Hybridization ratios for the probes were log-averaged over each chromosome. Approximately 1800 probes were resident on the X chromosome in this microarray design. FIGS. 45 and 46 show the results of these experiments. The error bars in FIGS. 45 and 46 reflect one standard deviation of expected error in the mean of the log₁₀ ratios for the probes over each chromosome. The number of genome copies (starting cells) was 100 for FIG. 45 and 10 for FIG. 46. It was found, as expected, that departures from unity ratio for the normal chromosomes tend to be larger as the starting DNA amounts decrease. In both figures, the X aneuploidy is detected as a departure of several standard deviations, whereas the other chromosomes are not significantly different from unit ratio at a level of significance of two standard deviations.

In these experiments, the raw hybridization values actually showed larger errors, but these errors were consistent from experiment to experiment in terms of which chromosome regions tended to be biased high or low. When these systematic bias patterns were learned from a previous data set, and applied as a correction to the subject data set, the values shown in FIGS. 45 and 46 were obtained. This adaptive correction was done using singular value decomposition of the chromosome-averaged biases over the set of experiments, and was applied to the value of all but Chromosome X.

### Example 21

### Confirmation of the Presence of Male Fetal Cells in Enriched Samples

Confirmation of the presence of a male fetal cell in an enriched sample is performed using qPCR with primers specific for DYZ, a marker repeated in high copy number on the Y chromosome. After enrichment of fnRBC by any of the methods described herein, the resulting enriched fnRBC are binned by dividing the sample into 100 PCR wells. Prior to binning, enriched samples may be screened by FISH to determine the presence of any fnRBC containing an aneuploidy of interest. Because of the low number of fnRBC in maternal blood, only a portion of the wells will contain a single fnRBC (the other wells are expected to be negative for fnRBC). The cells are fixed in 2%. Paraformaldehyde and stored at 4° C. Cells in each bin are pelleted and resuspended in 5 μl PBS plus 1 μl 20 mg/ml Proteinase K (Sigma #P-2308). Cells are lysed by incubation at 65° C. for 60 minutes followed by inactivation of the Proteinase K by incubation for 15 minutes at 95° C. For each reaction, primer sets (DYZ forward primer TCGAGTGCATTCCATTCCG [SEQ ID NO: 46]; DYZ reverse primer ATGGAATGGCATCAAACGGAA [SEQ ID NO: 47]; and DYZ Taqman Probe 6FAM-TGGCCTGTCCATTCCA-MGBNFQ) [SEQ ID NO. 48], TaqMan Universal PCR master mix. No AmpErase and water are added. The samples are run and analysis is performed on an ABI 7300: 2 minutes at 50° C., 10 minutes 95° C. followed by 40 cycles of 95° C. (15 seconds) and 60° C. (1 minute). Following confirmation of the presence of male fetal cells, further analysis of bins containing fnRBC is performed. Positive bins may be pooled prior to further analysis.

FIG. 45 shows the results expected from such an experiment. The data in FIG. 45 was collected by the following protocol. Nucleated red blood cells were enriched from cord cell blood of a male fetus by sucrose gradient two Heme Extractions (HE). The cells were fixed in 2% paraformaldehyde and stored at 4° C. Approximately 10×1000 cells were pelleted and resuspended each in 5 μl PBS plus 1 μl 20 mg/ml Proteinase K (Sigma #P-2308). Cells were lysed by incubation at 65° C. for 60 minutes followed by a inactivation of the Proteinase K by 15 minute at 95° C. Cells were combined and serially diluted 10-fold in PBS for 100, 10 and 1 cell per 6 μl final concentration were obtained, Six μl of each dilution was assayed in quadruplicate in 96 well format. For each reaction, primer sets were used as above (SEQ ID NOs: 45-48) and TaqMan Universal PCR master mix; No AmpErase and water were added to a final volume of 25 μl per reaction. Plates were run and analyzed on an ABI 7300: 2 minutes at 50° C., 10 minutes 95° C. followed by 40 cycles of 95° C. (15 seconds) and 60° C. (1 minute). These results show that detection of a single fnRBC in a bin is possible using this method.

### Example 22

### Confirmation of the Presence of Fetal Cells in Enriched Samples by STR Analysis

Maternal blood is processed through a size-based separation module, with or without subsequent MHEM enhancement of fnRBCs. The enhanced sample is then subjected to FISH analysis using probes specific to the aneuploidy of interest (e.g., triploidy 13, triploidy 18, and XYY). Individual positive cells are isolated by “plucking” individual positive cells from the enhanced sample using standard micromanipulation techniques. Using a nested PCR protocol, STR marker sets are amplified and analyzed to confirm that the FISH-positive aneuploid cell(s) are of fetal origin. For this analysis, comparison to the maternal genotype is typical. An example of a potential resulting data set is shown in Table 5. Non-maternal alleles may be proven to be paternal alleles by paternal genotyping or genotyping of known fetal tissue samples. As can be seen, the presence of paternal alleles in the resulting cells, demonstrates that the cell is of fetal origin (cells #1, 2, 9, and 10). Positive cells may be pooled for further analysis to diagnose aneuploidy of the fetus, or may be further analyzed individually.

TABLE 5
STR locus alleles in maternal and fetal cells

| | STR | STR | STR | STR | STR |
|---|---|---|---|---|---|
| | locus | locus | locus | locus | locus |
| DNA Source | D14S | D16S | D8S | F13B | vWA |
| Maternal alleles | 14, 17 | 11, 12 | 12, 14 | 9, 9 | 16, 17 |
| Cell #1 alleles | | 8 | | | 19 |
| Cell #2 alleles | 17 | | 15 | | |
| Cell #3 alleles | | | 14 | | |
| Cell #4 alleles | | | | | |
| Cell #5 alleles | 17 | 12 | | 9 | |
| Cell #6 alleles | | | | | |
| Cell #7 alleles | | | | | 19 |
| Cell #8 alleles | | | | | |
| Cell #9 alleles | 17 | | 14 | 7, 9 | 17, 19 |
| Cell #10 alleles | | | 15 | | |

### Example 23

### Confirmation of the Presence of Fetal Cells in Enriched Samples by SNP Analysis

Maternal blood is processed through a size-based separation module, with or without subsequent MHEM enhancement of fnRBCs. The enhanced sample is then subjected to FISH analysis using probes specific to the aneuploidy of interest (e.g., triploidy 13, triploidy 18, and XYY). Samples testing positive with FISH analysis are then binned into 96 microtiter wells, each well containing 15 μl of the enhanced sample. Of the 96 wells, 5-10 are expected to contain a single fnRBC and each well should contain approximately 1000 nucleated maternal cells (both WBC and mnRBC). Cells are pelleted and resuspended in 5 μl PBS plus 1 μl 20 mg/ml Proteinase K (Sigma #P-2308). Cells are lysed by incubation at 65° C. for 60 minutes followed by a inactivation of the Proteinase K by 15 minute at 95° C.

In this example, the maternal genotype (BB) and fetal genotype (AB) for a particular set of SNPs is known. The genotypes A and B encompass all three SNPs and differ from each other at all three SNPs. The following sequence from chromosome 7 contains these three SNPs (rs7795605, rs7795611 and rs7795233 indicated in brackets, respectively) (ATGCAGCAAGGCACAGACTAA[G/A]CAAGGAGA[G/C]GCAAAATTTTC[A/G]TAGGGGAGAGAAATGGGTCATT) [SEQ ID NO: 49].

In the first round of PCR, genomic DNA from binned enriched cells is amplified using primers specific to the outer portion of the fetal-specific allele A and which flank the interior SNP (forward primer ATGCAGCAAGGCACAGACTACG [SEQ ID NO: 50]; reverse primer AGAGGGGAGAGAAATGGGTCATT [SEQ ID NO: 51]). In the second round of PCR, amplification using real time SYBR Green PCR is performed with primers specific to the inner portion of allele A and which encompass the interior SNP (forward primer CAAGGCACAGACTAAGCAAGGACGAG [SEQ ID NO: 52]; reverse primer GGCAAAATTTTCATAGCGGGAGAGAAATGGGTCATT [SEQ ID NO: 53]).

Expected results are shown in FIG. 46. Here, six of the 96 wells test positive for allele A, confirming the presence of cells of fetal origin, because the maternal genotype (BB) is known and cannot be positive for allele A. DNA from positive wells may be pooled for further analysis or analyzed individually.

While the invention has been described hereinabove with reference to a preferred embodiment and various alternatives thereto, it should be apparent that the invention is not limited to such embodiment(s). Rather, many variations would be apparent to persons of skill in the art without departing from the scope and spirit of this invention, as defined in the appended claims.

While preferred embodiments of the present invention have been shown and described herein, it can be obvious to those skilled in the art that such embodiments are provided by way of example only. Numerous variations, changes, and substitutions can occur without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for determining if fetal cell is present in a sample comprising: determining a ratio of expression levels of two or more marker genes in a cell present in said sample; and determining a fetal cell is present based on said ratio of marker genes.

2. A method for genetic testing comprising: enriching a cellular component from a cell; determining in situ an expression level for two or more marker genes in said component to obtain a ratio of said two or more marker genes; identifying a nuclei in which said two or more marker genes are expressed; isolating said nuclei by microdissection; and conducting genetic testing using said nuclei.

3. A method for obtaining genetic information from a cell present in a blood sample comprising: enriching a cell present in a blood sample comprising treating said blood sample to render hemoglobin present in said blood sample magnetically responsive; obtaining a ratio of expression levels for two or more marker genes; identifying a fetal cell or nucleus is present based on said ratio; and extracting genetic information from said fetal cell or said nucleus.

4. (canceled)

5. The method of claims 1, wherein said cell is a fetal red blood cell.

6. The method of claims 1, wherein said expression levels correspond to ribosomal RNA, mRNA or nascent RNA transcripts.

7. The method of claims 2, wherein said enriching comprises, size based separation, administering a magnetic field, selective cell lysis or a combination of any thereof.

8. The method of claims 1, wherein said sample is a maternal blood sample.

9. The methods of claims 1, wherein said marker genes are differentially expressed during fetal development.

10. The method of claim 12, wherein said marker genes encode hemoglobins.

11. The method of claims 1, wherein said two or more marker genes are selected from a group consisting of DYS1, SRY, DYZ, CD-71, ε-globin, γ-globin, ζ-globin, α-globin and β-globin.

12. The method of claims 1, wherein at least one of said two or more marker genes is a negative selection marker for a fetal cell.

13. The method of claims 1, wherein at least one of said two or more marker genes is a positive selection marker for a fetal cell.

14. The method of claim 4, wherein said FISH comprises conducting RNA or chromosomal FISH.

15. The method of claim 14, wherein said chromosomal FISH comprises conducting at least X and Y chromosome FISH.

16. The method of claim 14, wherein said chromosomal FISH comprises conducting Y FISH with at least two different Y-chromosome specific probes.

17. (canceled)

18. The method of claim 17, wherein said single chromosome is selected from a group of chromosomes consisting of 13, 18, 21 and X.

19. The method of claim 11, wherein said ratio is of γ-/β-hemoglobin provides a numerical index.

20. The method of claim 19, wherein an index of at least about 5 indicates positively that a fetal cell(s) is present in said sample.

21. The method of claims 19, wherein an index of less than about 1.5 indicates positively that a fetal cell(s) is not present in said sample.

22. The method of claims 1, wherein said marker is a Y chromosome-specific marker.

23. The method of claims 1, wherein said marker is DYZ.

24. (canceled)

25. (canceled)

26. The method of claims 1, further comprising: performing nested PCR or whole genome amplification to amplify a target sequence of interest; analyzing short tandem repeat (STR(s)) analysis using capillary electrophoresis; and performing comparative genome hybridization or microarray analysis, thereby obtaining genetic information from the cell of interest.

27. The method of claim 26, wherein said determining comprises measuring expression levels of at least two hemoglobin genes.

28. The method of claim 26, wherein said determining comprises measuring a ratio of expression levels for γ-hemoglobin over β-hemoglobin to obtain an index whereby if said index is greater than about 1 then said cells are identified as fetal nucleated red blood cells.

29. The method of claim 26, wherein said nested PCR comprises STR(s).

30. The method of claim 26, said STR(s) are located in non-coding regions of the gene sequence.

31. The method of claim 26, wherein said genetic information comprises disease status, gender, paternity, aneuploidy, or presence or absence of a mutation.

32. The method of claim 26, wherein said target genes are highly expressed.

33. (canceled)

34. The method of claim 26, wherein said short tandem repeat is a Y-chromosome specific marker.

35. The method of claim 26, wherein said short tandem repeat is DYZ.

36. (canceled)

37. A kit for amplification of fetal biomarkers comprising reverse and forward primers for γ and β globin and reverse transcriptase.

38. A kit for amplification of fetal biomarkers comprising reverse and forward primers for ε and β globin and reverse transcriptase.

39. A method for determining if a fetal cell is present in a sample comprising: enriching a fetal cell by size based selection from a maternal blood sample and detecting ε-globin positive cells; wherein said ε-globin positive cells are designated as fetal cells and ε-globin negative cells are designated as maternal cells.

40. A method for determining if a fetal cell is present in a sample comprising: enriching a fetal cell by size based selection from a maternal blood sample and detecting γ-globin positive cells; wherein said γ-globin positive cells are designated as fetal cells and γ-globin negative cells are designated as maternal cells.

41. The method of claim 39, wherein said enriching fetal cell by size based selection comprises enrichment with a microfluidic device.

42. The method of claim 40, wherein said enriching fetal cell by size based selection comprises enrichment with a microfluidic device.

43. The method of claim 39, wherein said microfluidic device comprises an array of obstacles.

44. The method of claim 39, wherein said enriching fetal cell by size based selection comprises magnetic enrichment of fetal cells, wherein said fetal cells comprise magnetically responsive hemoglobin.


SEQUENCE LISTING (102 sequences)

SEQ ID NO: 1 - DNA, 30 bp, Artificial Sequence
  Description of Artificial Sequence Synthetic probe
cgacctggacttttgccaggcacagggtcc

SEQ ID NO: 2 - DNA, 30 bp, Artificial Sequence
  Description of Artificial Sequence Synthetic probe
tcactcccaaccccagtatcttcaaacagc

SEQ ID NO: 3 - DNA, 30 bp, Artificial Sequence
  Description of Artificial Sequence Synthetic probe
gcatctttttaacgaccatacttgtcctgc

SEQ ID NO: 4 - DNA, 30 bp, Artificial Sequence
  Description of Artificial Sequence Synthetic probe
acagagctgactttcaaaatctactccagc

SEQ ID NO: 5 - DNA, 30 bp, Artificial Sequence
  Description of Artificial Sequence Synthetic probe
ttccacactgatgcaatcattcgtctgttt

SEQ ID NO: 6 - DNA, 30 bp, Artificial Sequence
  Description of Artificial Sequence Synthetic probe
tgtgtacacatattaaaacattacacttta

SEQ ID NO: 7 - DNA, 30 bp, Artificial Sequence
  Description of Artificial Sequence Synthetic probe
attagcaatatgaaacctcttacatcagtt

SEQ ID NO: 8 - DNA, 30 bp, Artificial Sequence
  Description of Artificial Sequence Synthetic probe
agtaatgtactaggcagactgtgtaaagtt

SEQ ID NO: 9 - DNA, 35 bp, Artificial Sequence
  Description of Artificial Sequence Synthetic probe
ctggactaggagcttattgataacctcagacgttc

SEQ ID NO: 10 - DNA, 36 bp, Artificial Sequence
  Description of Artificial Sequence Synthetic probe
gcagaataaagcctatccttgaaagctctgaatcat

SEQ ID NO: 11 - DNA, 36 bp, Artificial Sequence
  Description of Artificial Sequence Synthetic probe
agggaacaaaggaacctttaatagaaattggacagc

SEQ ID NO: 12 - DNA, 29 bp, Artificial Sequence
  Description of Artificial Sequence Synthetic probe
tttgaggttgctagtgaacacagttgtgt

SEQ ID NO: 13 - DNA, 35 bp, Artificial Sequence; identical to SEQ ID NO: 9
  Description of Artificial Sequence Synthetic probe

SEQ ID NO: 14 - DNA, 36 bp, Artificial Sequence; identical to SEQ ID NO: 10
  Description of Artificial Sequence Synthetic probe

SEQ ID NO: 15 - DNA, 36 bp, Artificial Sequence; identical to SEQ ID NO: 11
  Description of Artificial Sequence Synthetic probe

SEQ ID NO: 16 - DNA, 29 bp, Artificial Sequence
  Description of Artificial Sequence Synthetic probe
tttgaggttgctagtgaacacagttgtgt

SEQ ID NO: 17 - DNA, 30 bp, Artificial Sequence
  Description of Artificial Sequence Synthetic probe
cctggtcaccagagcctaccttcccagggt

SEQ ID NO: 18 - DNA, 28 bp, Artificial Sequence
  Description of Artificial Sequence Synthetic probe
cttgtaaccttgataccaacctgcccag

SEQ ID NO: 19 - DNA, 30 bp, Artificial Sequence; identical to SEQ ID NO: 17
  Description of Artificial Sequence Synthetic probe

SEQ ID NO: 20 - DNA, 28 bp, Artificial Sequence
  Description of Artificial Sequence Synthetic probe
cttgtaaccttgataccaacctgcccag

SEQ ID NO: 21 - DNA, 30 bp, Artificial Sequence; identical to SEQ ID NO: 1
  Description of Artificial Sequence Synthetic probe

SEQ ID NO: 22 - DNA, 30 bp, Artificial Sequence; identical to SEQ ID NO: 2
  Description of Artificial Sequence Synthetic probe

SEQ ID NO: 23 - DNA, 30 bp, Artificial Sequence; identical to SEQ ID NO: 3
  Description of Artificial Sequence Synthetic probe

SEQ ID NO: 24 - DNA, 30 bp, Artificial Sequence; identical to SEQ ID NO: 4
  Description of Artificial Sequence Synthetic probe

SEQ ID NO: 25 - DNA, 30 bp, Artificial Sequence; identical to SEQ ID NO: 5
  Description of Artificial Sequence Synthetic probe

SEQ ID NO: 26 - DNA, 30 bp, Artificial Sequence; identical to SEQ ID NO: 6
  Description of Artificial Sequence Synthetic probe

SEQ ID NO: 27 - DNA, 30 bp, Artificial Sequence; identical to SEQ ID NO: 7
  Description of Artificial Sequence Synthetic probe

SEQ ID NO: 28 - DNA, 30 bp, Artificial Sequence; identical to SEQ ID NO: 8
  Description of Artificial Sequence Synthetic probe

SEQ ID NO: 29 - DNA, 35 bp, Artificial Sequence; identical to SEQ ID NO: 9
  Description of Artificial Sequence Synthetic probe

SEQ ID NO: 30 - DNA, 36 bp, Artificial Sequence; identical to SEQ ID NO: 10
  Description of Artificial Sequence Synthetic probe

SEQ ID NO: 31 - DNA, 36 bp, Artificial Sequence; identical to SEQ ID NO: 11
  Description of Artificial Sequence Synthetic probe

SEQ ID NO: 32 - DNA, 29 bp, Artificial Sequence
  Description of Artificial Sequence Synthetic probe
tttgaggttgctagtgaacacagttgtgt

SEQ ID NO: 33 - DNA, 35 bp, Artificial Sequence; identical to SEQ ID NO: 9
  Description of Artificial Sequence Synthetic probe

SEQ ID NO: 34 - DNA, 36 bp, Artificial Sequence; identical to SEQ ID NO: 10
  Description of Artificial Sequence Synthetic probe

SEQ ID NO: 35 - DNA, 36 bp, Artificial Sequence; identical to SEQ ID NO: 11
  Description of Artificial Sequence Synthetic probe

SEQ ID NO: 36 - DNA, 29 bp, Artificial Sequence
  Description of Artificial Sequence Synthetic probe
tttgaggttgctagtgaacacagttgtgt

SEQ ID NO: 37 - DNA, 30 bp, Artificial Sequence; identical to SEQ ID NO: 17
  Description of Artificial Sequence Synthetic probe

SEQ ID NO: 38 - DNA, 28 bp, Artificial Sequence
  Description of Artificial Sequence Synthetic probe
cttgtaaccttgataccaacctgcccag

SEQ ID NO: 39 - DNA, 30 bp, Artificial Sequence; identical to SEQ ID NO: 17
  Description of Artificial Sequence Synthetic probe

SEQ ID NO: 40 - DNA, 28 bp, Artificial Sequence
  Description of Artificial Sequence Synthetic probe
cttgtaaccttgataccaacctgcccag

SEQ ID NO: 41 - DNA, 20 bp, Artificial Sequence
  Description of Artificial Sequence Synthetic primer
tgcagcttgtcacagtgcag

SEQ ID NO: 42 - DNA, 26 bp, Artificial Sequence
  Description of Artificial Sequence Synthetic oligonucleotide
gagccaggccatcactaaaggcaccg

SEQ ID NO: 43 - DNA, 25 bp, Artificial Sequence
  Description of Artificial Sequence Synthetic oligonucleotide
ctttgtggcatctcccaaggaagtc

SEQ ID NO: 44 - DNA, 26 bp, Artificial Sequence
  Description of Artificial Sequence Synthetic probe
gagccaggccatcactaaaggcaccg

SEQ ID NO: 45 - DNA, 25 bp, Artificial Sequence
  Description of Artificial Sequence Synthetic probe
ctttgtggcatctcccaaggaagtc

SEQ ID NO: 46 - DNA, 19 bp, Artificial Sequence
  Description of Artificial Sequence Synthetic primer
tcgagtgcattccattccg

SEQ ID NO: 47 - DNA, 21 bp, Artificial Sequence
  Description of Artificial Sequence Synthetic primer
atggaatggcatcaaacggaa

SEQ ID NO: 48 - DNA, 15 bp, Artificial Sequence
  Description of Artificial Sequence Synthetic probe
tggctgtccattcca

SEQ ID NO: 49 - DNA, 65 bp, Homo sapiens
atgcagcaaggcacagactaarcaaggagasgcaaaattttcrtaggggagagaaatgggtcatt

SEQ ID NO: 50 - DNA, 22 bp, Artificial Sequence
  Description of Artificial Sequence Synthetic primer
atgcagcaaggcacagactacg

SEQ ID NO: 51 - DNA, 23 bp, Artificial Sequence
  Description of Artificial Sequence Synthetic primer
agaggggagagaaatgggtcatt

SEQ ID NO: 52 - DNA, 25 bp, Artificial Sequence
  Description of Artificial Sequence Synthetic primer
caaggcacagactaagcaaggagag

SEQ ID NO: 53 - DNA, 35 bp, Artificial Sequence
  Description of Artificial Sequence Synthetic primer
ggcaaaattttcataggggagagaaatgggtcatt

SEQ ID NO: 54 - DNA, 19 bp, Artificial Sequence
  Description of Artificial Sequence Synthetic primer
tggacccagaggtttttga

SEQ ID NO: 55 - DNA, 32 bp, Artificial Sequence
  Description of Artificial Sequence Synthetic primer
ttctaatatgcaaatgcacacagatttctgct

SEQ ID NO: 56 - DNA, 32 bp, Artificial Sequence
  Description of Artificial Sequence Synthetic primer
ttcagattcagactgaatgacaccatcagttt

SEQ ID NO: 57 - DNA, 24 bp, Artificial Sequence
  Description of Artificial Sequence Synthetic primer
ttggagtcgcaagctgaactagcg

SEQ ID NO: 58 - DNA, 24 bp, Artificial Sequence
  Description of Artificial Sequence Synthetic primer
ccaggaagttgaggctgcagtgaa

SEQ ID NO: 59 - DNA, 32 bp, Artificial Sequence
  Description of Artificial Sequence Synthetic primer
taaagtgagaaagaataactgcatcttaacct

SEQ ID NO: 60 - DNA, 27 bp, Artificial Sequence
  Description of Artificial Sequence Synthetic primer
tctcctttctcttcctcatccctgcat

SEQ ID NO: 61 - DNA, 27 bp, Artificial Sequence
  Description of Artificial Sequence Synthetic primer
gctctggaaaacaactcgacccttctt

SEQ ID NO: 62 - DNA, 22 bp, Artificial Sequence
  Description of Artificial Sequence Synthetic primer
gtgggagaatcgcctgagtcct

SEQ ID NO: 63 - DNA, 30 bp, Artificial Sequence
  Description of Artificial Sequence Synthetic primer
gtctgttatgggacttttctcagtctccat

SEQ ID NO: 64 - DNA, 36 bp, Artificial Sequence
  Description of Artificial Sequence Synthetic primer
acactgagaagggagaaacactgtaaggttttatat

SEQ ID NO: 65 - DNA, 20 bp, Artificial Sequence
  Description of Artificial Sequence Synthetic primer
gctagattttccccgatgat

SEQ ID NO: 66 - DNA, 23 bp, Artificial Sequence
  Description of Artificial Sequence Synthetic primer
atgtaaagtgctctcaagagtgc

SEQ ID NO: 67 - DNA, 25 bp, Artificial Sequence
  Description of Artificial Sequence Synthetic primer
gcatgcacctgtagttccagctact

SEQ ID NO: 68 - DNA, 22 bp, Artificial Sequence
  Description of Artificial Sequence Synthetic primer
gagagcaacgtgtccctcctgt

SEQ ID NO: 69 - DNA, 31 bp, Artificial Sequence
  Description of Artificial Sequence Synthetic primer
cagaagagactgcccttcagactttctaaat

SEQ ID NO: 70 - DNA, 25 bp, Artificial Sequence
  Description of Artificial Sequence Synthetic primer
gtacacgcctgtaatcccagctact

SEQ ID NO: 71 - DNA, 32 bp, Artificial Sequence
  Description of Artificial Sequence Synthetic primer
tacacctttaaaattccaaagaaagttcttct

SEQ ID NO: 72 - DNA, 29 bp, Artificial Sequence
  Description of Artificial Sequence Synthetic primer
caattctgcttctcagatcctctgacact

SEQ ID NO: 73 - DNA, 19 bp, Artificial Sequence
  Description of Artificial Sequence Synthetic primer
ccaaggcccttcccaggct

SEQ ID NO: 74 - DNA, 28 bp, Artificial Sequence
  Description of Artificial Sequence Synthetic primer
tgacactgctacaactcacaccacattt

SEQ ID NO: 75 - DNA, 21 bp, Artificial Sequence
  Description of Artificial Sequence Synthetic primer
aacccatgttcccactggcct

SEQ ID NO: 76 - DNA, 27 bp, Artificial Sequence
  Description of Artificial Sequence Synthetic primer
caaacgtgaggttgactctactgtcct

SEQ ID NO: 77 - DNA, 31 bp, Artificial Sequence
  Description of Artificial Sequence Synthetic primer
agactgatcctataaggtagagttcccacct

SEQ ID NO: 78 - DNA, 36 bp, Artificial Sequence
  Description of Artificial Sequence Synthetic primer
tagagacaggatagatgataaatagatacataggtt

SEQ ID NO: 79 - DNA, 23 bp, Artificial Sequence
  Description of Artificial Sequence Synthetic primer
ttggagtcgcaagctgaactagc

SEQ ID NO: 80 - DNA, 23 bp, Artificial Sequence
  Description of Artificial Sequence Synthetic primer
gcctgagtgacagagtgagaacc

SEQ ID NO: 81 - DNA, 27 bp, Artificial Sequence
  Description of Artificial Sequence Synthetic primer
tgtaataactctacgactgtctgtctg

SEQ ID NO: 82 - DNA, 21 bp, Artificial Sequence
  Description of Artificial Sequence Synthetic primer
gaataggaggtggatggatgg

SEQ ID NO: 83 - DNA, 18 bp, Artificial Sequence
  Description of Artificial Sequence Synthetic primer
gtgagtcaattccccaag

SEQ ID NO: 84 - DNA, 22 bp, Artificial Sequence
  Description of Artificial Sequence Synthetic primer
gttgtattagtcaatgttctcc

SEQ ID NO: 85 - DNA, 23 bp, Artificial Sequence
  Description of Artificial Sequence Synthetic primer
attttccccgatgatagtagtct

SEQ ID NO: 86 - DNA, 26 bp, Artificial Sequence
  Description of Artificial Sequence Synthetic primer
gcgaatgtatgattggcaatattttt

SEQ ID NO: 87 - DNA, 20 bp, Artificial Sequence
  Description of Artificial Sequence Synthetic primer
tgaggtggtgtactaccata

SEQ ID NO: 88 - DNA, 20 bp, Artificial Sequence
  Description of Artificial Sequence Synthetic primer
gatcatgccattgcactcta

SEQ ID NO: 89 - DNA, 24 bp, Artificial Sequence
  Description of Artificial Sequence Synthetic primer
ccctagtggatgataagaataatc

SEQ ID NO: 90 - DNA, 30 bp, Artificial Sequence
  Description of Artificial Sequence Synthetic primer
ggacagatgataaatacataggatggatgg

SEQ ID NO: 91 - DNA, 24 bp, Artificial Sequence
  Description of Artificial Sequence Synthetic primer
ttccacacaccactggccatcttc

SEQ ID NO: 92 - DNA, 24 bp, Artificial Sequence
  Description of Artificial Sequence Synthetic primer
aacctgagtctgccaaggactagc

SEQ ID NO: 93 - DNA, 25 bp, Artificial Sequence
  Description of Artificial Sequence Synthetic primer
ggtaagcaggtacttagttagctac

SEQ ID NO: 94 - DNA, 24 bp, Artificial Sequence
  Description of Artificial Sequence Synthetic primer
gttacagtgagccaaggtcgtgag

SEQ ID NO: 95 - DNA, 24 bp, Artificial Sequence
  Description of Artificial Sequence Synthetic primer
gaggttgcactcgagcctttgcaa

SEQ ID NO: 96 - DNA, 24 bp, Artificial Sequence
  Description of Artificial Sequence Synthetic primer
ttcctgaatcatcccagagccaca

SEQ ID NO: 97 - DNA, 28 bp, Artificial Sequence
  Description of Artificial Sequence Synthetic primer
attatccaaaagtcaaatgccccatagg

SEQ ID NO: 98 - DNA, 28 bp, Artificial Sequence
  Description of Artificial Sequence Synthetic primer
atcgaaaatatggttattgaagtagctg

SEQ ID NO: 99 - DNA, 24 bp, Artificial Sequence
  Description of Artificial Sequence Synthetic primer
gtgggctgaaaagctcccgattat

SEQ ID NO: 100 - DNA, 24 bp, Artificial Sequence
  Description of Artificial Sequence Synthetic primer
attcaaagggtatctgggctctgg

SEQ ID NO: 101 - DNA, 24 bp, Artificial Sequence
  Description of Artificial Sequence Synthetic primer
actggcacagaacaggcacttagg

SEQ ID NO: 102 - DNA, 24 bp, Artificial Sequence
  Description of Artificial Sequence Synthetic primer
ggaggaactgggaaccacacaggt